SEQUENCE LISTING (152 sequences)

SEQ ID NO: 1 - DNA, 1107 bp, Homo sapiens
  CDS (65)..(598)
aattcggtacgaggctggggttcaggcgggcagcagctgcaggctgaccttgcagcttggcggaatggactggcctcacaacctgctgtttcttcttaccatttccatcttcctggggctgggcagccaggagccccaaaagcaagaggaaggggcaagggcggcctgggccctggcctggcctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctgaatcacctggcccagaagccaggccagcagcccgagaccatcctccttgcacctttgtgccaagaaaggcctatgaaaagtaaacactgacttttgaaagcaaaaaaaccccaggaagcttcggctgggttccagacacatggaaaacagacttcctgtgccagcgcatgctgatcccttcagcagccgcttctccacccttggggctgctctccagcacctggcagtgtccagagcggataggggcgccgtgtttggtgaatgagtgcacagacgcctctagggggagcccaagatctgcctcctgcctccctctattatgccttcataggtgggtcagaacaaagaattccttatcaacctcccgggtcccccactgccaatcacccacctccattctaccctctacagctgccccttatcccccaaagtcctgaaattttgcttgggtcacctgctccaggaggcagagttcccatgaagggtattaaacgtctactacactgc

SEQ ID NO: 2 - protein, 178 aa, Homo sapiens
MDWPHNLLFLLTISIFLGLGSQEPQKQEEGARAAWALAWPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 3 - DNA, 1603 bp, Homo sapiens
  CDS (92)..(1123)
caagcttgagagcaacacaatctatcaggaaagaaagaaagaaaaaaaccgaacctgacaaaaaagaagaaaaagaagaagaaaaaaaatcatgaaaaccatccagccaaaaatgcacaattctatctcttgggcaatcttcacggggctggctgctctgtgtctcttccaaggagtgcccgtgcgcagcggagatgccaccttccccaaagctatggacaacgtgacggtccggcagggggagagcgccaccctcaggtgcactattgacaaccgggtcacccgggtggcctggctaaaccgcagcaccatcctctatgctgggaatgacaagtggtgcctggatcctcgcgtggtccttctgagcaacacccaaacgcagtacagcatcgagatccagaacgtggatgtgtatgacgagggcccttacacctgctcggtgcagacagacaaccacccaaagacctctagggtccacctcattgtgcaagtatctcccaaaattgtagagatttcttcagatatctccattaatgaagggaacaatattagcctcacctgcatagcaactggtagaccagagcctacggttacttggagacacatctctcccaaagcggttggctttgtgagtgaagacgaatacttggaaattcagggcatcacccgggagcagtcaggggactacgagtgcagtgcctccaatgacgtggccgcgcccgtggtacggagagtaaaggtcaccgtgaactatccaccatacatttcagaagccaagggtacaggtgtccccgtgggacaaaaggggacactgcagtgtgaagcctcagcagtcccctcagcagaattccagtggtacaaggatgacaaaagactgattgaaggaaagaaaggggtgaaagtggaaaacagacctttcctctcaaaactcatcttcttcaatgtctctgaacatgactatgggaactacacttgcgtggcctccaacaagctgggccacaccaatgccagcatcatgctatttggtccaggcgccgtcagcgaggtgagcaacggcacgtcgaggagggcaggctgcgtctggctgccgcctcttctggtcttgcacctgcttctcaaattttgatgtgagtgccacttccccacccgggaaaggctgccgccaccaccaccaccaacacaacagcaatggcaacaccgacagcaaccaatcagatatatacaaatgaaattagaagaaacacagcctcatgggacagaaatttgagggaggggaacaaagaatactttggggggaaaagagttttaaaaaagaaattgaaaattgccttgcagatatttaggtacaatggagttttcttttcccaaacgggaagaacacagcacacccggcttggacccactgcaagctgcatcgtgcaacctctttggtgccagtgtgggcaagggctcagcctctctgcccacagagtgcccccacgtggaacattctggagctggccatcccaaattcaatcagtccatagagacgaacagaatgagaccttccggcccaagcgtggcgctgcgggcactttggtagactgtgccaccacggcgtgtg

SEQ ID NO: 4 - protein, 344 aa, Homo sapiens
MKTIQPKMHNSISWAIFTGLAALCLFQGVPVRSGDATFPKAMDNVTVRQGESATLRCTIDNRVTRVAWLNRSTILYAGNDKWCLDPRVVLLSNTQTQYSIEIQNVDVYDEGPYTCSVQTDNHPKTSRVHLIVQVSPKIVEISSDISINEGNNISLTCIATGRPEPTVTWRHISPKAVGFVSEDEYLEIQGITREQSGDYECSASNDVAAPVVRRVKVTVNYPPYISEAKGTGVPVGQKGTLQCEASAVPSAEFQWYKDDKRLIEGKKGVKVENRPFLSKLIFFNVSEHDYGNYTCVASNKLGHTNASIMLFGPGAVSEVSNGTSRRAGCVWLPPLLVLHLLLKF

SEQ ID NO: 5 - DNA, 2012 bp, Homo sapiens
  CDS (501)..(1532)
gccagggaatgccagggggaaagggattttctgatactcagaagactcagagactgtcagtttaaaaaatgaaagtaatatagaaggggcaaagtggcatttatcattctatctctccaggctcctgtctctttaatcagctagcctgatttgcccagtaaatgattcctgagagtgtgtgtgcgtgtgtgtgtgtgtgtgtgcccgcgcgcgtgtgttgtagctctgtcaatccttggattagaaccaatgattgcagcttgtaagagggctgtccagggccagattgtacaatgtgtctcagtgccagagtatgagtggagataattacggagaagtcatactctctcacaccctcggctttcttgttgtgtccttcagcaaaacagtggatttaaatctccttgcacaagcttgagagcaacacaatctatcaggaaagaaagaaagaaaaaaaccgaacctgacaaaaaagaagaaaaagaagaagaaaaaaaatcatgaaaaccatccagccaaaaatgcacaattctatctcttgggcaatcttcacggggctggctgctctgtgtctcttccaaggagtgcccgtgcgcagcggagatgccaccttccccaaagctatggacaacgtgacggtccggcagggggagagcgccaccctcaggtgcactattgacaaccgggtcacccgggtggcctggctaaaccgcagcaccatcctctatgctgggaatgacaagtggtgcctggatcctcgcgtggtccttctgagcaacacccaaacgcagtacagcatcgagatccagaacgtggatgtgtatgacgagggcccttacacctgctcggtgcagacagacaaccacccaaagacctctagggtccacctcattgtgcaagtatctcccaaaattgtagagatttcttcagatatctccattaatgaagggaacaatattagcctcacctgcatagcaactggtagaccagagcctacggttacttggagacacatctctcccaaagcggttggctttgtgagtgaagacgaatacttggaaattcagggcatcacccgggagcagtcaggggactacgagtgcagtgcctccaatgacgtggccgcgcccgtggtacggagagtaaaggtcaccgtgaactatccaccatacatttcagaagccaagggtacaggtgtccccgtgggacaaaaggggacactgcagtgtgaagcctcagcagtcccctcagcagaattccagtggtacaaggatgacaaaagactgattgaaggaaagaaaggggtgaaagtggaaaacagacctttcctctcaaaactcatcttcttcaatgtctctgaacatgactatgggaactacacttgcgtggcctccaacaagctgggccacaccaatgccagcatcatgctatttggtccaggcgccgtcagcgaggtgagcaacggcacgtcgaggagggcaggctgcgtctggctgccgcctcttctggtcttgcacctgcttctcaaattttgatgtgagtgccacttccccacccgggaaaggctgccgccaccaccaccaccaacacaacagcaatggcaacaccgacagcaaccaatcagatatatacaaatgaaattagaagaaacacagcctcatgggacagaaatttgagggaggggaacaaagaatactttggggggaaaagagttttaaaaaagaaattgaaaattgccttgcagatatttaggtacaatggagttttcttttcccaaacgggaagaacacagcacacccggcttggacccactgcaagctgcatcgtgcaacctctttggtgccagtgtgggcaagggctcagcctctctgcccacagagtgcccccacgtggaacattctggagctggccatcccaaattcaatcagtccatagagacgaacagaatgagaccttccggcccaagcgtggcgctgcgggcactttggtagactgtgccaccacggcgtgtg

SEQ ID NO: 6 - protein, 344 aa, Homo sapiens; identical to SEQ ID NO: 4

SEQ ID NO: 7 - DNA, 1228 bp, Homo sapiens
  CDS (529)..(1026)
gctcttcctgaaggaagatccagtggcatatctccatggctgccagacagagtagagaaatggaacttatcggtgtctcttcagaagttttgttacaaatatccagaaatatttctataatctaatcagcagattatgaatatatgcattagactttagttttggtgcaatcacatgaattccattttgtggagtaagaggtgactggggtatagggtacaacccatagccatccatgttcatctttgttttgaatataattggctagaagatatacatatatctatgtaacttcctctagcatcctccagtatggaggctgcattaagactgcatgaaggagagggagagaagggagaaacagagcagctggacaagaggacaggtatagggaataagggagaagccagtaaggcaggaaagaccctccgtgacaaaggggcagggaacagaactcaaacatttaatggcaggtaacccaggttagaatggtaaattgaaaggtgaatataaagggagaatggtgaaatgaattttctgaaattaattgctgtgtttatagtttttagccatgcatcggaatcacctcaggactccactcccaatcaattatatatctgggggaggaccaaggcgttggtatttttcagaagctccactggtgattctgacagcacagctaggattaagaaactgatcaatgggaacggcatgcctgttgcagaggagcttccctgggaaatgtcacacacagaacatcaatcttccttccccactcctgagatccctcattctttggcaccaggaacagttgcaattagtaaaccctggttccctgctgtctcacaaatcgcaagagtccaacgtgtggatataaacttttgttcatgggaggatctttctcccagtggaaaagcaactgggaaaagcaggacacactgcacagtgactgcagtttcatccaatgccaccacccatgcaggcataaataatgaacatggatgggggagtctggagctgctgaattgaggaagaaagaacacagaaattaaaattctcacaaaggttaccattaagctagaggaagaccacaccactgtgtgtccacaaagatacagagccaggccgggttcagccatgctggtcatctgctctatataatacaattatttagagatggtgggtagagaacaactacagaaaaaaaaaaaaaaaaaaaaaaaaaaaa

SEQ ID NO: 8 - protein, 166 aa, Homo sapiens [sequence omitted]

SEQ ID NO: 9 - DNA, 1917 bp, Homo sapiens
  CDS (410)..(889)
acgcgtcacataaaggaaagatacgttttaatcatctttacaagtgcgtccttgtacctttcgggataacctgtactgatttctctgcaggaccttttcaaagaatcctcttcaagagagaaacaaattttaggctgacgacttcacggagaggcaggttctgctgttgccaatgaacgagaactttctactaggctggcggcatgcagagcccacgtctgtcagctgccaccttcgtaaagcacacgtttcacatgcatgagctcgagtggctagaacttcaaaactgtgctcaggtttttgttttggaagttataaaaaagttgctcacaaacaatagttattgccttttatatcttttatgttagtctactagtcagcattctgcccaaaatggaaagccactcccatgggaagggagggggtagcagctgggagtctgctcttccagctgggggccctcccacccccatggggaggaaagacgtcaagctccagccactggccccggtgggtcccaaagccccacccctcatgctctcctctggtcacctctatttacgctcacatgccccttcctgtccttcacctgcacgtcaccagcaggtcccgccaaccccaaatctatctggtgaaaacctggagaacaagagcggagtctaagagagatgtaaatgaaaacacagatcaacagacacaccagaagggaagcgttgtttccgcggggaaaggagatggaaaggggaagagaagtgaagaattctgcgcccgaagctcgggttggtgtttgctcaactgctttactcattttaaccctttcacctatcctgggagaaacccaggcttgtcaccttttcatgttgggttgtttgtttattggcctcttaagtgagaattgatccgtgaagggaaacagacaggaggaggtcagattgcgaatacctggggcttcctagggtccagtgcggcagttaccgcacctgccttcaccggtgaacctttagccagctgaacaaccaccaaagcgccctgcagagacaagtcatccagccctctggcatgtccctggtagcccgggcaccagccgctgcggcttgtgaggggcaccatgctccaccccacggggaccttcacagttggaaaaaagaagaggaaaaactaattccttcggtaacagtttattttcatttttgggaaaggcaaaaccactacctggaactcggtgcctccgtggttaactttcctattttgcttgtgatttaaaggctgttctgggtcaggggggaaaaggtgtctccttcggtagggaatatataacgtggtgataacctgtcactaggcagaagcatccactctgcagggacagtggcccctcaggaaagcccgccgctcctggccaaggcctctctgcagactccacgggggctcaccctctgccgtcaggcgactctgaaattccgacatttctcccttaaagtctcaacagacacaagagaagtttccatcaagcaagcactgacatatttatattaaaaaatagtgcaaaatctcaacatttatataaataactctaaacccctgctttgtaatttttttctttacaaggtaatacacactttctgacttggcactcaaaaattgccatttttttcctcttctagttcagaaaacaacttttttttttaataggcctcttctaatacaaaaatactcctgccctcgcacatacagtttctcttatcttatatatatttatatatataatattgcagatctttaaacaaaggttttgtgcaaatatgtctttaaagttaagtgaaattatcataaacaaaagaaaataagcattcacgcacgcagctcaactagaaacaagaaagactactgtagaaattttttttcttttgccttcaagac

SEQ ID NO: 10 - protein, 160 aa, Homo sapiens
MGREGVAAGSLLFQLGALPPPWGGKTSSSSHWPRWVPKPHPSCSPLVTSIYAHMPLPVLHLHVTSRSRQPQIYLVKTWRTRAESKRDVNENTDQQTHQKGSVVSAGKGDGKGKRSEEFCARSSGWCLLNCFTHFNPFTYPGRNPGLSPFHVGLFVYWPLK

SEQ ID NO: 11 - DNA, 1279 bp, Homo sapiens
  CDS (410)..(892)
  misc_feature (1226)..(1226): Wherein n may be a, c, g or t
  misc_feature (1229)..(1229): Wherein n may be a, c, g or t
  misc_feature (1230)..(1230): Wherein n may be a, c, g or t
  misc_feature (1236)..(1236): Wherein n may be a, c, g or t
  misc_feature (1237)..(1237): Wherein n may be a, c, g or t
  misc_feature (1239)..(1239): Wherein n may be a, c, g or t
  misc_feature (1242)..(1242): Wherein n may be a, c, g or t
  misc_feature (1243)..(1243): Wherein n may be a, c, g or t
  misc_feature (1246)..(1246): Wherein n may be a, c, g or t
  misc_feature (1249)..(1249): Wherein n may be a, c, g or t
  misc_feature (1251)..(1251): Wherein n may be a, c, g or t
  misc_feature (1252)..(1252): Wherein n may be a, c, g or t
  misc_feature (1255)..(1255): Wherein n may be a, c, g or t
  misc_feature (1256)..(1256): Wherein n may be a, c, g or t
  misc_feature (1258)..(1258): Wherein n may be a, c, g or t
  misc_feature (1259)..(1259): Wherein n may be a, c, g or t
  misc_feature (1261)..(1261): Wherein n may be a, c, g or t
  misc_feature (1262)..(1262): Wherein n may be a, c, g or t
  misc_feature (1263)..(1263): Wherein n may be a, c, g or t
  misc_feature (1264)..(1264): Wherein n may be a, c, g or t
  misc_feature (1266)..(1266): Wherein n may be a, c, g or t
  misc_feature (1267)..(1267): Wherein n may be a, c, g or t
  misc_feature (1268)..(1268): Wherein n may be a, c, g or t
  misc_feature (1269)..(1269): Wherein n may be a, c, g or t
  misc_feature (1270)..(1270): Wherein n may be a, c, g or t
  misc_feature (1272)..(1272): Wherein n may be a, c, g or t
  misc_feature (1273)..(1273): Wherein n may be a, c, g or t
  misc_feature (1277)..(1277): Wherein n may be a, c, g or t
  misc_feature (1278)..(1278): Wherein n may be a, c, g or t
acgcgtcacataaaggaaagatacgttttaatcatctttacaagtgcgtccttgtacctttcgggataacctgtactgatttctctgcaggaccttttcaaagaatcctcttcaagagagaaacaaattttaggctgacgacttcacggagaggcaggttctgctgttgccaatgaacgagaactttctactaggctggcggcatgcagagcccacgtctgtcagctgccaccttcgtaaagcacacgtttcacatgcatgagctcgagtggctagaacttcaaaactgtgctcaggtttttgttttggaagttataaaaaagttgctcacaaacaatagttattgccttttatatcttttatgttagtctactagtcagcattctgcccaaaatggaaagccactcccatgggaagggagggggtagcagctgggagtctgctcttccagctgggggccctcccacccccatggggaggaaagacgtcaagctccagccactggccccggtgggtcccaaagccccacccctcatgctctcctctggtcacctctatttacgctcacatgccccttcctgtccttcacctgcacgtcaccagcaggtcccgccaaccccaaatctatctggtgaaaacctggagaacaagagcggagtctaagagagatgtaaatgaaaacacagatcaacagacacaccagaagggaagcgttgtttccgcggggaaaggagatggaaaggggaagagaagtgaagaattctgcgcccgaagctcgggttggtgtttgctcaactgctttactcattttaaccctttcacctatcctgggagaaacccaggcttgtcaccttttcatgttgggttgtttattggcctcttaagtgagaattgatccgtgaagggaaacagacaggaggaggtcagattgcgaatacctggggcttcctagggtccagtgcggcagttaccgcacctgccttcaccggtgaacctttagccagctgaacaaccaccaaagcgccctgcagagacaagtcatcgagccctctggcatgtccctggtagcccgggcaccagccgctgcggcttgtgaggggcaccatgctccaccccacggggaccttcacagttggaaaaaagaagaggaaaaactaattccttcggtaacagtttattttcatttttgggaaaggcaaaaccactacctggaactcggtgcctgnganntcttanntnctnnctnagncnnatnngnnannnntnnnnnanncttnna

SEQ ID NO: 12 - protein, 161 aa, Homo sapiens
MGREGVAAGSLLFQLGALPPPWGGKTSSSSHWPRWVPKPHPSCSPLVTSIYAHMPLPVLHLHVTSRSRQPQIYLVKTWRTRAESKRDVNENTDQQTHQKGSVVSAGKGDGKGKRSEEFCARSSGWCLLNCFTHFNPFTYPGRNPGLSPFHVGLFIGLLSEN

SEQ ID NO: 13 - DNA, 1689 bp, Homo sapiens
  CDS (199)..(1146)
tagaattcagcggccgcttaattctagaacgaatgccagtgcctggaggcatgcaggcccagctacgtgcctgtgtgcggctctgatgggaggttttatgaaaaccactgtaagctccaccgtgctgcttgcctcctgggaaagaggatcaccgtcatccacagcaaggactgtttcctcaaaggtgacacgtgcaccatggccggctacgcccgcttgaagaatgtccttctggcactccagacccgtctgcagccactccaagaaggagacagcagacaagaccctgcctcccagaagcgcctcctggtggaatctctgttcagggacttagatgcagatggcaatggccacctcagcagctccgaactggctcagcatgtgctgaagaagcaggacctggatgaagacttacttggttgctcaccaggtgacctcctccgatttgacgattacaacagtgacagctccctgaccctccgcgagttctacatggccttccaagtggttcagctcagcctcgcccccgaggacagggtcagtgtgaccacagtgaccgtggggctgagcacagtgctgacctgcgccgtccatggagacctgaggccaccaatcatctggaagcgcaacgggctcaccctgaacttcctggacttggaagacatcaatgactttggagaggatgattccctgtacatcaccaaggtgaccaccatccacatgggcaattacacctgccatgcttccggccacgagcagctgttccagacccacgtcctgcaggtgaatgtgccgccagtcatccgtgtctatccagagagccaggcacaggagcctggagtggcagccagcctaagatgccatgctgagggcattcccatgcccagaatcacttggctgaaaaacggcgtggatgtctcaactcagatgtccaaacagctctcccttttagccaatgggagcgaactccacatcagcagtgttcggtatgaagacacaggggcatacacctgcattgccaaaaatgaagtgggtgtggatgaagatatctcctcgctcttcattgaagactcagctagaaagacccttgcaaacatcctgtggcgagaggaaggtaccaagcttcattgttttgcgtcatgcctgtgatcacgtgtgtttggttctatgatgggccgtctttccatgatctgccaccagctttcccacacaaagcagccctatgggagcaggaagtcaatgtcaaattcaagtggcatatgcattgaatcaaatttaaaatgtactcctgtctttaatgagaaatttttaaatgcaaagctttcattaaaagtggcttgtaacctctgctgaagcagaacagttggtaagggttcctggtcagatctgggccttaaacttttttccagtagctgactggtgttgggtttagtgttttgcctatcttgtgtggttttaaaaagacaaaacaagttgtagatctctactagatagtcactgtaccttaaatatgctttgattgaggaaaacccgaggaaaaagctgccatgatttctgccaatgtatatttttaaatgtatagatgtttagaaacatatttatcaagcaaatctttagtaagttgagccatatgaagttgccatttttgtgcatcaaagtggtctaagattgacaatttcatatggctga

SEQ ID NO: 14 - protein, 316 aa, Homo sapiens
MAGYARLKNVLLALQTRLQPLQEGDSRQDPASQKRLLVESLFRDLDADGNGHLSSSELAQHVLKKQDLDEDLLGCSPGDLLRFDDYNSDSSLTLREFYMAFQVVQLSLAPEDRVSVTTVTVGLSTVLTCAVHGDLRPPIIWKRNGLTLNFLDLEDINDFGEDDSLYITKVTTIHMGNYTCHASGHEQLFQTHVLQVNVPPVIRVYPESQAQEPGVAASLRCHAEGIPMPRITWLKNGVDVSTQMSKQLSLLANGSELHISSVRYEDTGAYTCIAKNEVGVDEDISSLFIEDSARKTLANILWREEGTKLHCFASCL

SEQ ID NO: 15 - DNA, 5573 bp, Homo sapiens
  CDS (408)..(2933)
ggagagggctgcattgctgttgctcactgaccttcttttatgctggcctttggttcagaatggcacatcattcctcgtttttggccctccagctgaacacctgttctctgtggcactgactcctctttccatagggacatcatacaacagtcgcctttatctgaggttgtgcaaagagggatggaggagaaaacaatggagaatccctggcagatttccccaggacgagagaaggatatccaattgctcatcagggaaggtgctaggtctcccagccagacgccctcagaggccggtgtcaagtctccctcacctctgtgatgtgaagtcagctcgttcatgacctgggcaggcagagggtcagaggggcagatggagcactcctggcctgatgaagactcatcaaaatgaaaccaggaggcttttggctgcatctcacactgctcggagcctccctgccggctgcgctgggatggatggacccaggaaccagcagaggcccggatgtgggtgtgggggagtcacaggcagaggagcccagaagctttgaagtcacaagaagagaagggctttccagccacaacgagctgctggcctcctgcgggaagaagttctgcagccgagggagccggtgcgtgctcagcaggaagacaggggagcccgaatgccagtgcctggaggcatgcaggcccagctacgtgcctgtgtgcggctctgatgggaggttttatgaaaaccactgtaagctccaccgtgctgcttgcctcctgggaaagaggatcaccgtcatccacagcaaggactgtttcctcaaaggtgacacgtgcaccatggccggctacgcccgcttgaagaatgtccttctggcactccagacccgtctgcagccactccaagaaggagacagcagacaagaccctgcctcccagaagcgcctcctggtggaatctctgttcagggacttagatgcagatggcaatggccacctcagcagctccgaactggctcagcatgtgctgaagaagcaggacctggatgaagacttacttggttgctcaccaggtgacctcctccgatttgacgattacaacagtgacagctccctgaccctccgcgagttctacatggccttccaagtggttcagctcagcctcgcccccgaggacagggtcagtgtgaccacagtgaccgtggggctgagcacagtgctgacctgcgccgtccatggagacctgaggccaccaatcatctggaagcgcaacgggctcaccctgaacttcctggacttggaagacatcaatgactttggagaggatgattccctgtacatcaccaaggtgaccaccatccacatgggcaattacacctgccatgcttccggccacgagcagctgttccagacccacgtcctgcaggtgaatgtgccgccagtcatccgtgtctatccagagagccaggcacaggagcctggagtggcagccagcctaagatgccatgctgagggcattcccatgcccagaatcacttggctgaaaaacggcgtggatgtctcaactcagatgtccaaacagctctcccttttagccaatgggagcgaactccacatcagcagtgttcggtatgaagacacaggggcatacacctgcattgccaaaaatgaagtgggtgtggatgaagatatctcctcgctcttcattgaagactcagctagaaagacccttgcaaacatcctgtggcgagaggaaggcctcagcgtgggaaacatgttctatgtcttctccgacgacggtatcatcgtcatccatcctgtggactgtgagatccagaggcacctcaaacccacggaaaagattttcatgagctatgaagaaatctgtcctcaaagagaaaaaaatgcaacccagccctgccagtgggtatctgcagtcaatgtccggaaccggtacatctatgtggcccagccagcactgagcagagtccttgtggtcgacatccaagcccagaaagtcctacagtccataggtgtggaccctctgccggctaagctgtcctatgacaagtcacatgaccaagtgtgggtcctgagctggggggacgtgcacaagtcccgaccaagtctccaggtgatcacagaagccagcaccggccagagccagcacctcatccgcacaccctttgcaggagtggatgatttcttcattcccccaacaaacctcatcatcaaccacatcaggtttggcttcatcttcaacaagtctgatcctgcagtccacaaggtggacctggaaacaatgatgcccctcaagaccatcggcctgcaccaccatggctgcgtgccccaggccatggcacacacccacctgggcggctacttcttcatccagtgccgacaggacagccccgcctctgctgcccgacagctgctcgttgacagtgtcacagactctgtgcttggccccaatggtgatgtaacaggcaccccacacacatcccccgacgggcgcttcatagtcagtgctgcagctgacagcccctggctgcacgtgcaggagatcacagtgcggggcgagatccagaccctgtatgacctgcaaataaactcgggcatctcagacttggccttccagcgctccttcactgaaagcaatcaatacaacatctacgcggctctgcacacggagccggacctgctgttcctggagctgtccacggggaaggtgggcatgctgaagaacttaaaggagccacccgcagggccagctcagccctgggggggtacccacagaatcatgagggacagtgggctgtttggacagtacctcctcacaccagcccgagagtcactgttcctcatcaatgggagacaaaacacgctgcggtgtgaggtgtcaggtataaagggggggaccacagtggtgtgggtgggtgaggtatgaagggcccagagcagagccctgggccaaggaacaccccctagtcctgacactgcagcctcaagcaggtacgctgtacatttttacagacaaaagcaaaaacctgtactcgctttgtggttcaacactggtctccttgcaagtttcctagtataaggtatgcgctgctaccaagattggggttttttcgttaggaagtatgatttatgccttgagctacgatgagaacatatgctgctgtgtaaagggatcatttctgtgccaagctgcacaccgagtgacctggggacatcatggaaccaagggatcctgctctccaagcagacacctctgtcagttgccttcacatagtcattgtcccttactgccagacccagccagactttgccctgacggagtggcccggaagcagaggccgaccaggagcaggggcctccctcccgaactgaaagcccatccgtcctcgcgtgggaccgcatcttctccctcgcagctgcttcttgcttttctttccatttgacttgctgtaagcctgagggagagccaacaagacttactgcatcttgggggatggggaaatcactcactttattttggaaatttttgattaaaaaaaaattttataatctcaaatgctagtaagcagaaagatgctctccgaggtccaactatatccttccctgccttaggccgagtctcgggggtggtcacaaccccacatcccacagccagaaagaacaatggtcatctgagaatactggccctgtcgactattgccaccctgcttctccaagagcagaccaggccacctcatccgtaaggactcggttctgtgttgggaccccaaaaaaccagaacaagttctgtgtgcctcctttcagcacagaagggagacatctcattagtcaggtctggtaccccagattcagggcagactgggcttgcctggcaaggtatgggtggcctccaggctcaatgcagaaaccccaaggacacgagtggggccaggtgagttcctgaagctataccttttcaaaacagattttgttttcctacctgtggcccatccactcctctctggtaccccatccccgcatcagcactgcagagagaacacatttcggcgagggttttcttacccacattccccaatcaatacacacacactgcagaacccagaacagaaggccacaggctggcactactgcattctccttatgtgtctcaggctgtggtgactctcacatgggcatcgaagaagtacaacccacatagccctctggagaccgcctagatcagagactcagcaaaaacaggctcgccttccctctcccacatatgagtggaacttacatgtgtcctggtttgaatgatcattttgcaagccacacgggttgggagaggtggtctcaccacagacgtctttgctaatttggccaccttcacctactgacatgaccaggattttcctttgccattaaggaatgaactctttcaaggagaggaaaccctagactctgtgtcactctcaacacacacagctcctttcactcctgcctgactgccaagccacctgcatcccccgccccagatctcatgagatcaatcacttgtatgtctcacgcaacttggtccaccaaacgcctgtcccctgtaactcctaggggtgcgcctagacaggtacgtctgttttttattttaaaagatatgctatgtagatataagttgaggaagctcacctcaaaagcctagaatgcagtttcacagtagctgggatgcatggatgacccatctcacccctttttttttcctgcctcaatatcttgatatgttatgtttactcccaatctcccatttttaccactaaaattctccaactttcataaacttttttttggaaaaatttccattgtatcagcccctgacagaaaaaggatctctgagcctaaaggaggaaaagtcccaccaactaccagaccagaacacgagcccctctgggcagcaggattcctaagtcaaagaccagtttgacccaaactggccttttaaaataatcaggagtgacagagtcaacttctgcagcacctgcttctcccccactgtcccttccatcttggaatgtgtctaaaaaagcatagctgccctttgctgtcctcagagtgcatttcctggagacggcaggcttaggtctcactgacagcatgccagacacaactgaatcgaagcaggcctgaagcctaggtcagggtttcaggagtccagccccaggaggcaaagtcaccaatgcagggaggtaaatgccttttggcaggaaaaccaatagagttggttgggtggggagtcaggggtgggaggagaaggaggaagaggaggaaggccagactggcctgccctttctcccatacttcaccccagcagaggttcatgggacacagttggaaagccactgggaggaaatgcctcactacaggggggcctcctgtagcaagcccagccggtaatcctcctaatgaacccacaaggtcaattcacaactgatatcttagctattaaagaagtactgactttaccaaaagaatcatcaagaaagctatttatataaaccccctcagtcattttgaaataaaattaattttacaa

SEQ ID NO: 16 - protein, 842 aa, Homo sapiens
MKPGGFWLHLTLLGASLPAALGWMDPGTSRGPDVGVGESQAEEPRSFEVTRREGLSSHNELLASCGKKFCSRGSRCVLSRKTGEPECQCLEACRPSYVPVCGSDGRFYENHCKLHRAACLLGKRITVIHSKDCFLKGDTCTMAGYARLKNVLLALQTRLQPLQEGDSRQDPASQKRLLVESLFRDLDADGNGHLSSSELAQHVLKKQDLDEDLLGCSPGDLLRFDDYNSDSSLTLREFYMAFQVVQLSLAPEDRVSVTTVTVGLSTVLTCAVHGDLRPPIIWKRNGLTLNFLDLEDINDFGEDDSLYITKVTTIHMGNYTCHASGHEQLFQTHVLQVNVPPVIRVYPESQAQEPGVAASLRCHAEGIPMPRITWLKNGVDVSTQMSKQLSLLANGSELHISSVRYEDTGAYTCIAKNEVGVDEDISSLFIEDSARKTLANILWREEGLSVGNMFYVFSDDGIIVIHPVDCEIQRHLKPTEKIFMSYEEICPQREKNATQPCQWVSAVNVRNRYIYVAQPALSRVLVVDIQAQKVLQSIGVDPLPAKLSYDKSHDQVWVLSWGDVHKSRPSLQVITEASTGQSQHLIRTPFAGVDDFFIPPTNLIINHIRFGFIFNKSDPAVHKVDLETMMPLKTIGLHHHGCVPQAMAHTHLGGYFFIQCRQDSPASAARQLLVDSVTDSVLGPNGDVTGTPHTSPDGRFIVSAAADSPWLHVQEITVRGEIQTLYDLQINSGISDLAFQRSFTESNQYNIYAALHTEPDLLFLELSTGKVGMLKNLKEPPAGPAQPWGGTHRIMRDSGLFGQYLLTPARESLFLINGRQNTLRCEVSGIKGGTTVVWVGEV

SEQ ID NO: 17 - DNA, 5502 bp, Homo sapiens
  CDS (420)..(2864)
caatttcacacaggaaacagctatgccatgattacgcaagttggtaccgagctcggatccactagtaacggccgccagtgtgctggaattcggcttactcactatagggctcgagcggctgcccgggcaggtcattaattccatttctttttagagtatcacagctttctccttcactgaccaccctttgcttcctgtcagaaagccctggacagaactctctgtgggattctgcccatgtttctgagatatcgcctcaattgtcctggctgggctgtcgggtctgcccgttttacagatgggcaaactggagtgggaagtatccgggtggcttcctcaggcctgcagctggtggagcagctactgaaacaatcaggagcccagaagctttgaagtcacaagaagagaagactcccagaatgcagtgtgatgttggtgatggacgcctgtttcgcctttcacttaaacgtgccctttccagctgccctgacctctttgggctttccagccgcaacgagctgctggcctcctgcgggaagaagttctgcagccgagggagccggtgcgtgctcagcaggaagacaggggagcccgaatgccagtgcctggaggcatgcaggcccagctacgtgcctgtgtgcggctctgatgggaggttttatgaaaaccactgtaagctccaccgtgctgcttgcctcctgggaaagaggatcaccgtcatccacagcaaggactgtttcctcaaaggtgacacgtgcaccatggccggctacgcccgcttgaagaatgtccttctggcactccagacccgtctgcagccactccaagaaggagacagcagacaagaccctgcctcccagaagcgcctcctggtggaatctctgttcagggacttagatgcagatggcaatggccacctcagcagctccgaactggctcagcatgtgctgaagaagcaggacctggatgaagacttacttggttgctcaccaggtgacctcctccgatttgacgattacaacagtgacagctccctgaccctccgcgagttctacatggccttccaagtggttcagctcagcctcgcccccgaggacagggtcagtgtgaccacagtgaccgtggggctgagcacagtgctgacctgcgccgtccatggagacctgaggccaccaatcatctggaagcgcaacgggctcaccctgaacttcctggacttggaagacatcaatgactttggagaggatgattccctgtacatcaccaaggtgaccaccatccacatgggcaattacacctgccatgcttccggccacgagcagctgttccagacccacgtcctgcaggtgaatgtgccgccagtcatccgtgtctatccagagagccaggcacaggagcctggagtggcagccagcctaagatgccatgctgagggcattcccatgcccagaatcacttggctgaaaaacggcgtggatgtctcaactcagatgtccaaacagctctcccttttagccaatgggagcgaactccacatcagcagtgttcggtatgaagacacaggggcatacacctgcattgccaaaaatgaagtgggtgtggatgaagatatctcctcgctcttcattgaagactcagctagaaagacccttgcaaacatcctgtggcgagaggaaggcctcagcgtgggaaacatgttctatgtcttctccgacgacggtatcatcgtcatccatcctgtggactgtgagatccagaggcacctcaaacccacggaaaagattttcatgagctatgaagaaatctgtcctcaaagagaaaaaaatgcaacccagccctgccagtgggtatctgcagtcaatgtccggaaccggtacatctatgtggcccagccagcactgagcagagtccttgtggtcgacatccaagcccagaaagtcctacagtccataggtgtggaccctctgccggctaagctgtcctatgacaagtcacatgaccaagtgtgggtcctgagctggggggacgtgcacaagtcccgaccaagtctccaggtgatcacagaagccagcaccggccagagccagcacctcatccgcacaccctttgcaggagtggatgatttcttcattcccccaacaaacctcatcatcaaccacatcaggtttggcttcatcttcaacaagtctgatcctgcagtccacaaggtggacctggaaacaatgatgcccctcaagaccatcggcctgcaccaccatggctgcgtgccccaggccatggcacacacccacctgggcggctacttcttcatccagtgccgacaggacagccccgcctctgctgcccgacagctgctcgttgacagtgtcacagactctgtgcttggccccaatggtgatgtaacaggcaccccacacacatcccccgacgggcgcttcatagtcagtgctgcagctgacagcccctggctgcacgtgcaggagatcacagtgcggggcgagatccagaccctgtatgacctgcaaataaactcgggcatctcagacttggccttccagcgctccttcactgaaagcaatcaatacaacatctacgcggctctgcacacggagccggacctgctgttcctggagctgtccacggggaaggtgggcatgctgaagaacttaaaggagccacccgcagggccagctcagccctgggggggtacccacagaatcatgagggacagtgggctgtttggacagtacctcctcacaccagcccgagagtcactgttcctcatcaatgggagacaaaacacgctgcggtgtgaggtgtcaggtataaagggggggaccacagtggtgtgggtgggtgaggtatgaagggcccagagcagagccctgggccaaggaacaccccctagtcctgacactgcagcctcaagcaggtacgctgtacatttttacagacaaaagcaaaaacctgtactcgctttgtggttcaacactggtctccttgcaagtttcctagtataaggtatgcgctgctaccaagattggggttttttcgttaggaagtatgatttatgccttgagctacgatgagaacatatgctgctgtgtaaagggatcatttctgtgccaagctgcacaccgagtgacctggggacatcatggaaccaagggatcctgctctccaagcagacacctctgtcagttgccttcacatagtcattgtcccttactgccagacccagccagactttgccctgacggagtggcccggaagcagaggccgaccaggagcaggggcctccctcccgaactgaaagcccatccgtcctcgcgtgggaccgcatcttctccctcgcagctgcttcttgcttttctttccatttgacttgctgtaagcctgagggagagccaacaagacttactgcatcttgggggatggggaaatcactcactttattttggaaatttttgattaaaaaaaaattttataatctcaaatgctagtaagcagaaagatgctctccgaggtccaactatatccttccctgccttaggccgagtctcgggggtggtcacaaccccacatcccacagccagaaagaacaatggtcatctgagaatactggccctgtcgactattgccaccctgcttctccaagagcagaccaggccacctcatccgtaaggactcggttctgtgttgggaccccaaaaaaccagaacaagttctgtgtgcctcctttcagcacagaagggagacatctcattagtcaggtctggtaccccagattcagggcagactgggcttgcctggcaaggtatgggtggcctccaggctcaatgcagaaaccccaaggacacgagtggggccaggtgagttcctgaagctataccttttcaaaacagattttgttttcctacctgtggcccatccactcctctctggtaccccatccccgcatcagcactgcagagagaacacatttcggcgagggttttcttacccacattccccaatcaatacacacacactgcagaacccagaacagaaggccacaggctggcactactgcattctccttatgtgtctcaggctgtggtgactctcacatgggcatcgaagaagtacaacccacatagccctctggagaccgcctagatcagagactcagcaaaaacaggctcgccttccctctcccacatatgagtggaacttacatgtgtcctggtttgaatgatcattttgcaagccacacgggttgggagaggtggtctcaccacagacgtctttgctaatttggccaccttcacctactgacatgaccaggattttcctttgccattaaggaatgaactctttcaaggagaggaaaccctagactctgtgtcactctcaacacacacagctcctttcactcctgcctgactgccaagccacctgcatcccccgccccagatctcatgagatcaatcacttgtatgtctcacgcaacttggtccaccaaacgcctgtcccctgtaactcctaggggtgcgcctagacaggtacgtctgttttttattttaaaagatatgctatgtagatataagttgaggaagctcacctcaaaagcctagaatgcagtttcacagtagctgggatgcatggatgacccatctcacccctttttttttcctgcctcaatatcttgatatgttatgtttactcccaatctcccatttttaccactaaaattctccaactttcataaacttttttttggaaaaatttccattgtatcagcccctgacagaaaaaggatctctgagcctaaaggaggaaaagtcccaccaactaccagaccagaacacgagcccctctgggcagcaggattcctaagtcaaagaccagtttgacccaaactggccttttaaaataatcaggagtgacagagtcaacttctgcagcacctgcttctcccccactgtcccttccatcttggaatgtgtctaaaaaagcatagctgccctttgctgtcctcagagtgcatttcctggagacggcaggcttaggtctcactgacagcatgccagacacaactgaatcgaagcaggcctgaagcctaggtcagggtttcaggagtccagccccaggaggcaaagtcaccaatgcagggaggtaaatgccttttggcaggaaaaccaatagagttggttgggtggggagtcaggggtgggaggagaaggaggaagaggaggaaggccagactggcctgccctttctcccatacttcaccccagcagaggttcatgggacacagttggaaagccactgggaggaaatgcctcactacaggggggcctcctgtagcaagcccagccggtaatcctcctaatgaacccacaaggtcaattcacaactgatatcttagctattaaagaagtactgactttaccaaaagaatcatcaagaaagctatttatataaaccccctcagtcattttgaaataaaattaattttac

SEQ ID NO: 18 - protein, 815 aa, Homo sapiens
MQCDVGDGRLFRLSLKRALSSCPDLFGLSSRNELLASCGKKFCSRGSRCVLSRKTGEPECQCLEACRPSYVPVCGSDGRFYENHCKLHRAACLLGKRITVIHSKDCFLKGDTCTMAGYARLKNVLLALQTRLQPLQEGDSRQDPASQKRLLVESLFRDLDADGNGHLSSSELAQHVLKKQDLDEDLLGCSPGDLLRFDDYNSDSSLTLREFYMAFQVVQLSLAPEDRVSVTTVTVGLSTVLTCAVHGDLRPPIIWKRNGLTLNFLDLEDINDFGEDDSLYITKVTTIHMGNYTCHASGHEQLFQTHVLQVNVPPVIRVYPESQAQEPGVAASLRCHAEGIPMPRITWLKNGVDVSTQMSKQLSLLANGSELHISSVRYEDTGAYTCIAKNEVGVDEDISSLFIEDSARKTLANILWREEGLSVGNMFYVFSDDGIIVIHPVDCEIQRHLKPTEKIFMSYEEICPQREKNATQPCQWVSAVNVRNRYIYVAQPALSRVLVVDIQAQKVLQSIGVDPLPAKLSYDKSHDQVWVLSWGDVHKSRPSLQVITEASTGQSQHLIRTPFAGVDDFFIPPTNLIINHIRFGFIFNKSDPAVHKVDLETMMPLKTIGLHHHGCVPQAMAHTHLGGYFFIQCRQDSPASAARQLLVDSVTDSVLGPNGDVTGTPHTSPDGRFIVSAAADSPWLHVQEITVRGEIQTLYDLQINSGISDLAFQRSFTESNQYNIYAALHTEPDLLFLELSTGKVGMLKNLKEPPAGPAQPWGGTHRIMRDSGLFGQYLLTPARESLFLINGRQNTLRCEVSGIKGGTTVVWVGEV

SEQ ID NO: 19 - DNA, 1208 bp, Homo sapiens
  CDS (124)..(1089)
ctttgcttcagccgcagtcgccactggctgcctgaggtgctcttacagcctgttccaagtgtggcttaatccgtctccaccaccagatctttctccgtggattcctctgctaagaccgctgccatgccagtgacggtaacccgcaccaccatcacaaccaccacgacgtcatcttcgggcctggggtcccccatgatcgtggggtcccctcgggccctgacacagcccctgggtctccttcgcctgctgcagctggtgtctacctgcgtggccttctcgctggtggctagcgtgggcgcctggacggggtccatgggcaactggtccatgttcacctggtgcttctgcttctccgtgaccctgatcatcctcatcgtggagctgtgcgggctccaggcccgcttccccctgtcttggcgcaacttccccatcaccttcgcctgctatgcggccctcttctgcctctcggcctccatcatctaccccaccacctatgtccagttcctgtcccacggccgttcgcgggaccacgccatcgccgccaccttcttctcctgcatcgcgtgtgtggcttacgccaccgaagtggcctggacccgggcccggcccggcgagatcactggctatatggccaccgtacccgggctgctgaaggtgctggagaccttcgttgcctgcatcatcttcgcgttcatcagcgaccccaacctgtaccagcaccagccggccctggagtggtgcgtggcggtgtacgccatctgcttcatcctagcggccatcgccatcctgctgaacctgggggagtgcaccaacgtgctacccatccccttccccagcttcctgtcggggctggccttgctgtctgtcctcctctatgccaccgcccttgttctctggcccctctaccagttcgatgagaagtatggcggccagcctcggcgctcgagagatgtaagctgcagccgcagccatgcctactacgtgtgtgcctgggaccgccgactggctgtggccatcctgacggccatcaacctactggcgtatgtggctgacctggtgcactctgcccacctggtttttgtcaaggtctaagactctcccaagaggctcccgttccctctccaacctctttgttcttcttgcccgagttttctttatggagtacttctttcctccgcctttcctctgttttcctcttcctgtctccc

SEQ ID NO: 20 - protein, 322 aa, Homo sapiens
MPVTVTRTTITTTTTSSSGLGSPMIVGSPRALTQPLGLLRLLQLVSTCVAFSLVASVGAWTGSMGNWSMFTWCFCFSVTLIILIVELCGLQARFPLSWRNFPITFACYAALFCLSASIIYPTTYVQFLSHGRSRDHAIAATFFSCIACVAYATEVAWTRARPGEITGYMATVPGLLKVLETFVACIIFAFISDPNLYQHQPALEWCVAVYAICFILAAIAILLNLGECTNVLPIPFPSFLSGLALLSVLLYATALVLWPLYQFDEKYGGQPRRSRDVSCSRSHAYYVCAWDRRLAVAILTAINLLAYVADLVHSAHLVFVKV

SEQ ID NO: 21 - DNA, 1203 bp, Homo sapiens
  CDS (587)..(1012)
ggaagaagaaggaggaggaggagaaggagaagaagaaggagaagaacgcaagacttcgtctcaaaaaaaaagaagaaaaaatttaaatacatttaaaaaagaaggttgcatgctgtggagcaaccagacaattgtgatgaaatgtgaagcacaaggcaccagctgtgacgtgtttttgccaagaagtcaaaccacgttccaactaaacctctagagcaaactttcattttcagcaaattcgaagaaaagaggaataatgtaaatgaccccacagggaaacagacaaaccctgaatgtggagcatttcacaggacaaaacctggacagacatcggaacacttacaggatgtgtgtagtgtggcatgacagagaactttggtttcctttaatgtgactgtagacctggcagtgttactataagaatcactggcaatcagacacccgggtgtgctgagctggcactcagtgggggcggctactgctcatgtgattgtggagtagacagttggaagaagtacccagtccatttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccaggtgagtggacatttgcatgccatctttatgaataagatttatctgtggatcatattaaaggtactgattgttctcatctctgacttccctaattatagccctggaggagggccactaagacctaaagtttaacaggccccattggtgatgctcagtgatatttaacaccttctctctgttttaaaactcatgggtgtgcctgggcgtggtggctcacacctct

SEQ ID NO: 22 - protein, 142 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPGEWTFACHLYE

SEQ ID NO: 23 - DNA, 825 bp, Homo sapiens
  CDS (494)..(769)
tctagaacattctccagccctttttttcttttgctcttttatgacattgacatgaagagtccgggccagttgttctggatttgtctgattgcttctccctggttggagtcaggtggaacagctctggcaggaacgcccccccgggcaatgcagagtcctcctccaggaggcacttagtgtccatgcgtcaccttgctggtgatgcttcactggatcacttggttccggggttgtccgcacgtctccctgtagtgcaggtgctccttcctctttccaattagcctgtgggatgggacttggaagctgtgtctgttctgctccactggcaaccttttcttcaatgacttaagctggtgttttgccattttccatactctatcatggggagtgttcagtatcggcatctagagatctcccctggccccatcacagctagagctatgctgtcccctttcagggacatcttgtaatttatccacccagcccccaactgatggacataaaggctgtctccccatcatctcctgctactacagacagcactgcagggactgtcctgctgtgtttttttaaggcatgggtactccagaagcagttgctcagctgcacccccaaggttgagtggaagtccctcggtaaaggaggaggagagagtgtgaagggaatggcaaggcggggagggagacagggcacaggtgtcctggcaacagcagatgggaaacaggtctggctaaggtaccagaagccaacaagtcccagaaaggtcaagtgactttcccaaggtcacacagcaagttgatggcagagctgggtacaggactcaga

SEQ ID NO: 24 - protein, 92 aa, Homo sapiens [sequence omitted]

SEQ ID NO: 25 - DNA, 1099 bp, Homo sapiens
  CDS (83)..(889)
ctagaattcagcggccgctgaattctagtgcagagtgagcaagggccgcctcatccagcttctctctgagagccagggccacatggctcacctggtgaactccgtcagcgacatcctggatgccctgcagagggaccgggggctgggccggccccgcaacaaggccgaccttcagagagcgcctgcccggggaacccggccccggggctgtgccactggctcccggccccgagactgtctggacgtcctcctaagcggacagcaggacgatggcgtctactctgtctttcccacccactacccggccggcttccaggtgtactgtgacatgcgcacggacggcggcggctggacggtgtttcagcgccgggaggacggctccgtgaacttcttccggggctgggatgcgtaccgagacggctttggcaggctcaccggggagcactggctagggctcaagaggatccacgccctgaccacacaggctgcctacgagctgcacgtggacctggaggactttgagaatggcacggcctatgcccgctacgggagcttcggcgtgggcttgttctccgtggaccctgaggaagacgggtacccgctcaccgtggctgactattccggcactgcaggcgactccctcctgaagcacagcggcatgaggttcaccaccaaggaccgtgacagcgaccattcagagaacaactgtgccgccttctaccgcggtgcctggtggtaccgcaactgccacacgtccaacctcaatgggcagtacctgcgcggtgcgcacgcctcctatgccgacggcgtggagtggtcctcctggaccggctggcagtactcactcaagttctctgagatgaagatccggccggtccgggaggaccgctagaccggtgcaccttgtccttggccctgctggtccctgtcgccccatccccgaccccacctcactctttcgtgaatgttctccacccacctgtgcctggcggacccactctccagtagggaggggccgggccatccctgacacgaagctccctgggccggtgaagtcacacatcgccttctcgccgtccccaccccctccatttggcag

SEQ ID NO: 26 - protein, 269 aa, Homo sapiens
MAHLVNSVSDILDALQRDRGLGRPRNKADLQRAPARGTRPRGCATGSRPRDCLDVLLSGQQDDGVYSVFPTHYPAGFQVYCDMRTDGGGWTVFQRREDGSVNFFRGWDAYRDGFGRLTGEHWLGLKRIHALTTQAAYELHVDLEDFENGTAYARYGSFGVGLFSVDPEEDGYPLTVADYSGTAGDSLLKHSGMRFTTKDRDSDHSENNCAAFYRGAWWYRNCHTSNLNGQYLRGAHASYADGVEWSSWTGWQYSLKFSEMKIRPVREDR

SEQ ID NO: 27 - DNA, 1054 bp, Homo sapiens
  CDS (38)..(844)
ccgcctcatccagcttctctctgagagccagggccacatggctcacctggtgaactccgtcagcgacatcctggatgccctgcagagggaccgggggctgggccggccccgcaacaaggccgaccttcagagagcgcctgcccggggaacccggccccggggctgtgccactggctcccggccccgagactgtctggacgtcctcctaagcggacagcaggacgatggcgtctactctgtctttcccacccactacccggccggcttccaggtgtactgtgacatgcgcacggacggcggcggctggacggtgtttcagcgccgggaggacggctccgtgaacttcttccggggctgggatgcgtaccgagacggctttggcaggctcaccggggagcactggctagggctcaagaggatccacgccctgaccacacaggctgcctacgagctgcacgtggacctggaggactttgagaatggcacggcctatgcccgctacgggagcttcggcgtgggcttgttcgccgtggaccctgaggaagacgggcacccgctcaccgtggctgactattccggcactgcaggcgactccctcctgaagcacagcggcatgaggttcaccaccaaggaccgtgacagcgaccattcagagaacaactgtgccgccttctaccgcggtgcctggtggtaccgcaactgccacacgtccaacctcaatgggcagtacctgcgcggtgcgcacgcctcctatgccgacggcgtggagtggtcctcctggaccggctggcagtactcactcaagttctctgagatgaagatccggccggtccgggaggaccgctagaccggtgcaccttgtccttggccctgctggtccctgtcgccccatccccgaccccacctcactctttcgtgaatgttctccacccacctgtgcctggcggacccactctccagtagggaggggccgggccatccctgacacgaagctccctgggccggtgaagtcacacatcgccttctcgccgtccccaccccctccatttggcag

SEQ ID NO: 28 - protein, 269 aa, Homo sapiens
MAHLVNSVSDILDALQRDRGLGRPRNKADLQRAPARGTRPRGCATGSRPRDCLDVLLSGQQDDGVYSVFPTHYPAGFQVYCDMRTDGGGWTVFQRREDGSVNFFRGWDAYRDGFGRLTGEHWLGLKRIHALTTQAAYELHVDLEDFENGTAYARYGSFGVGLFAVDPEEDGHPLTVADYSGTAGDSLLKHSGMRFTTKDRDSDHSENNCAAFYRGAWWYRNCHTSNLNGQYLRGAHASYADGVEWSSWTGWQYSLKFSEMKIRPVREDR

SEQ ID NO: 29 - DNA, 498 bp, Homo sapiens
  CDS (1)..(498)
atgaattttctgaaattaattgctgtgtttatagtttttagccatgcatcggaatcacctcaggactccactcccaatcaattatatatctgggggaggaccaaggcgttggtatttttcagaagctccactggtgattctgacagcacagctaggattaagaaactgatcaatgggaacagcatgcctgttgcagaggagcttccctgggaaatgtcacacacagaacatcaatcttccttccccactcctgagatccctcattctttggcaccaggaacagttgcaattagtaaaccctggttccctgctgtctcacaaatcgcaagagtccaacgtgtggatataaacttttgttcatgggaggatctttctcccagtggaaaagcaactgggaaaagcaggacacactgcacagtgactgcagtttcatccaatgccaccacccatgcaggcataaataatgaacatggatgggggagtctggagctgctgaat

SEQ ID NO: 30 - protein, 166 aa, Homo sapiens
MNFLKLIAVFIVFSHASESPQDSTPNQLYIWGRTKALVFFRSSTGDSDSTARIKKLINGNSMPVAEELPWEMSHTEHQSSFPTPEIPHSLAPGTVAISKPWFPAVSQIARVQRVDINFCSWEDLSPSGKATGKSRTHCTVTAVSSNATTHAGINNEHGWGSLELLN

SEQ ID NO: 31 - DNA, 717 bp, Homo sapiens
cagagagcgcctgcccggggaacccggccccggggctgtgccactggctcccggccccgagactgtctggacgtcctcctaagcggacagcaggacgatggcgtctactctgtctttcccacccactacccggccggcttccaggtgtactgtgacatgcgcacggacggcggcggctggacggtgtttcagcgccgggaggacggctccgtgaacttcttccggggctgggacgcgtaccgagacggctttggcaggctcaccggggagcactggctagggctcaagaggatccacgccctgaccacacaggctgcctacgagctgcacgtggacctggaggactttgagaatggcacggcctatgcccgctacgggagcttcggcgtgggcttgttcgccgtggaccctgaggaagacgggtacccgctcaccgtggctgactattccggcactgcaggcgactccctcctgaagcacagcggcatgaggttcaccaccaaggaccgtgacagcgaccattcagagaacaactgtgccgccttctaccgcggtgcctggtggtaccgcaactgccacacgtccaacctcaatgggcagtacctgcgcggtgcgcacgcctcctatgccgacggcgtggagtggtcctcctggaccggctggcagtactcactcaagttctctgagatgaagatccggccggtccgggaggaccgc

SEQ ID NO: 32 - DNA, 30 bp, Artificial Sequence
  Description of Artifical Sequence: Primer/Probe
ctcgtcctcgagggtaagcctatccctaac

SEQ ID NO: 33 - DNA, 31 bp, Artificial Sequence
  Description of Artifical Sequence: Primer/Probe
ctcgtcgggcccctgatcagcgggtttaaac

SEQ ID NO: 34 - DNA, 40 bp, Artificial Sequence
  Description of Artifical Sequence: Primer/Probe
ctcgtcagatctcgcagcggagatgccaccttccccaaag

SEQ ID NO: 35 - DNA, 40 bp, Artificial Sequence
  Description of Artifical Sequence: Primer/Probe
ctcgtcctcgagcctcctcgacgtgccgttgctcacctcg

SEQ ID NO: 36 - DNA, 43 bp, Artificial Sequence
  Description of Artifical Sequence: Primer/Probe
ggatccaccatgaattttctgaaattaattgctgtgtttatag

SEQ ID NO: 37 - DNA, 36 bp, Artificial Sequence
  Description of Artifical Sequence: Primer/Probe
ctcgagattcagcagctccagactcccccatccatg

SEQ ID NO: 38 - DNA, 30 bp, Artificial Sequence
  Description of Artifical Sequence: Primer/Probe
agatctcagagagcgcctgcccggggaacc

SEQ ID NO: 39 - DNA, 31 bp, Artificial Sequence
  Description of Artifical Sequence: Primer/Probe
ctcgaggcggtcctcccggaccggccggatc

SEQ ID NO: 40 - DNA, 18 bp, Artificial Sequence
  Description of Artifical Sequence: Primer/Probe
gaggacggctccgtgaac

SEQ ID NO: 41 - DNA, 18 bp, Artificial Sequence
  Description of Artifical Sequence: Primer/Probe
gttcacggagccgtcctc

SEQ ID NO: 42 - DNA, 19 bp, Artificial Sequence
  Description of Artifical Sequence: Primer/Probe
cagcggcatgaggttcacc

SEQ ID NO: 43 - DNA, 19 bp, Artificial Sequence
  Description of Artifical Sequence: Primer/Probe
ggtgaacctcatgccgctg

SEQ ID NO: 44 - DNA, 14 bp, Artificial Sequence
  Description of Artifical Sequence: Primer/Probe
catggtcagcctac

SEQ ID NO: 45 - DNA, 14 bp, Artificial Sequence
  Description of Artifical Sequence: Primer/Probe
tcgagtaggctgac

SEQ ID NO: 46 - DNA, 20 bp, Artificial Sequence
  Description of Artifical Sequence: Primer/Probe
tccctgggaaatgtcacaca

SEQ ID NO: 47 - DNA, 21 bp, Artificial Sequence
  Description of Artifical Sequence: Primer/Probe
ttcctggtgccaaagaatgag

SEQ ID NO: 48 - DNA, 31 bp, Artificial Sequence
  Description of Artifical Sequence: Primer/Probe
agaacatcaatcttccttccccactcctgag

SEQ ID NO: 49 - DNA, 21 bp, Artificial Sequence
  Description of Artifical Sequence: Primer/Probe
gcctggctctctggatagaca

SEQ ID NO: 50 - DNA, 20 bp, Artificial Sequence
  Description of Artifical Sequence: Primer/Probe
cacgagcagctgttccagac

SEQ ID NO: 51 - DNA, 21 bp, Artificial Sequence
  Description of Artifical Sequence: Primer/Probe
tggcggcacattcacctgcag

SEQ ID NO: 52 - DNA, 23 bp, Artificial Sequence
  Description of Artifical Sequence: Primer/Probe
aaaggcggaggaaagaagtactc

SEQ ID NO: 53 - DNA, 18 bp, Artificial Sequence
  Description of Artifical Sequence: Primer/Probe
gctcccgttccctctcca

SEQ ID NO: 54 - DNA, 31 bp, Artificial Sequence
  Description of Artifical Sequence: Primer/Probe
cctctttgttcttcttgcccgagttttcttt

SEQ ID NO: 55 - DNA, 21 bp, Artificial Sequence
  Description of Artifical Sequence: Primer/Probe
tggaagtccctcggtaaagga

SEQ ID NO: 56 - DNA, 20 bp, Artificial Sequence
  Description of Artifical Sequence: Primer/Probe
aggacacctgtgccctgtct

SEQ ID NO: 57 - DNA, 21 bp, Artificial Sequence
  Description of Artifical Sequence: Primer/Probe
cccgccttgccattcccttca

SEQ ID NO: 58 - skipped

SEQ ID NO: 59 - DNA, 20 bp, Artificial Sequence
  Description of Artifical Sequence: Primer/Probe
tcttcatccaggtcctgctt

SEQ ID NO: 60 - DNA, 25 bp, Artificial Sequence
  Description of Artifical Sequence: Primer/Probe
cttcagcacatgctgagccagttcg

SEQ ID NO: 61 - DNA, 22 bp, Artificial Sequence
  Description of Artifical Sequence: Primer/Probe
ttcagggacttagatgcagatg

SEQ ID NO: 62 - DNA, 21 bp, Artificial Sequence
  Description of Artifical Sequence: Primer/Probe
ctgtactcgctttgtggttca

SEQ ID NO: 63 - DNA, 26 bp, Artificial Sequence
  Description of Artifical Sequence: Primer/Probe
cactggtctccttgcaagtttcctag

SEQ ID NO: 64 - DNA, 21 bp, Artificial Sequence
  Description of Artifical Sequence: Primer/Probe
aatcttggtagcagcgcatac

SEQ ID NO: 65 - DNA, 34 bp, Artificial Sequence
  Description of Artifical Sequence: Primer/Probe
aagctttcagaagtggaatacagagcggaggtcg

SEQ ID NO: 66 - DNA, 36 bp, Artificial Sequence
  Description of Artifical Sequence: Primer/Probe
ctcgagttcataaagatggcatgcaaatgtccactc

SEQ ID NO: 67 - DNA, 26 bp, Artificial Sequence
  Description of Artifical Sequence: Primer/Probe
gggggaatgaatagctcccctcactt

SEQ ID NO: 68 - DNA, 25 bp, Artificial Sequence
  Description of Artifical Sequence: Primer/Probe
actcactatagggctcgagcggctg

SEQ ID NO: 69 - DNA, 36 bp, Artificial Sequence
  Description of Artifical Sequence: Primer/Probe
ctcgagatgaaaccaggaggcttttggctgcatctc

SEQ ID NO: 70 - DNA, 35 bp, Artificial Sequence
  Description of Artifical Sequence: Primer/Probe
ctcgagtacctcacccacccacaccactgtggtcc

SEQ ID NO: 71 - DNA, 23 bp, Artificial Sequence
  Description of Artifical Sequence: Primer/Probe
gaaaaccactgtaagctccaccg

SEQ ID NO: 72 - DNA, 27 bp, Artificial Sequence
  Description of Artifical Sequence: Primer/Probe
aggtgacctcctccgatttgacgatta

SEQ ID NO: 73 - DNA, 25 bp, Artificial Sequence
  Description of Artifical Sequence: Primer/Probe
tacatcaccaaggtgaccaccatcc

SEQ ID NO: 74 - DNA, 25 bp, Artificial Sequence
  Description of Artifical Sequence: Primer/Probe
gaagatatctcctcgctcttcattg

SEQ ID NO: 75 - DNA, 25 bp, Artificial Sequence
  Description of Artifical Sequence: Primer/Probe
caagcccagaaagtcctacagtcca

SEQ ID NO: 76 - DNA, 23 bp, Artificial Sequence
  Description of Artifical Sequence: Primer/Probe
acaatgatgcccctcaagaccat

SEQ ID NO: 77 - DNA, 25 bp, Artificial Sequence
  Description of Artifical Sequence: Primer/Probe
caatcaatacaacatctacgcggct

SEQ ID NO: 78 - DNA, 25 bp, Artificial Sequence
  Description of Artifical Sequence: Primer/Probe
tttcataaaacctcccatcagagcc

SEQ ID NO: 79 - DNA, 27 bp, Artificial Sequence
  Description of Artifical Sequence: Primer/Probe
caagtaagtcttcatccaggtcctgct

SEQ ID NO: 80 - DNA, 23 bp, Artificial Sequence
  Description of Artifical Sequence: Primer/Probe
gtgtaattgcccatgtggatggt

SEQ ID NO: 81 - DNA, 25 bp, Artificial Sequence
  Description of Artifical Sequence: Primer/Probe
tatcttcatccacacccacttcatt

SEQ ID NO: 82 - DNA, 23 bp, Artificial Sequence
  Description of Artifical Sequence: Primer/Probe
gatgtcgaccacaaggactctgc

SEQ ID NO: 83 - DNA, 23 bp, Artificial Sequence
  Description of Artifical Sequence: Primer/Probe
ggtcttgaggggcatcattgttt

SEQ ID NO: 84 - DNA, 23 bp, Artificial Sequence
  Description of Artifical Sequence: Primer/Probe
tgattgctttcagtgaaggagcg

SEQ ID NO: 85 - DNA, 5573 bp, Homo sapiens; identical to SEQ ID NO: 15
  CDS (408)..(2933)

SEQ ID NO: 86 - DNA, 2538 bp, Homo sapiens
  CDS (1)..(2538)
ctcgagatgaaaccaggaggcttttggctgcatctcacactgctcggagcctccctgccggctgcgctgggatggatggacccaggaaccagcagaggcccggatgtgggtgtgggggagtcacaggcagaggagcccagaagctttgaagtcacaagaagagaagggctttccagccacaacgagctgctggcctcctgcgggaagaagttctgcagccgagggagccggtgcgtgctcagcaggaagacaggggagcccgaatgccagtgcctggaggcatgcaggcccagctacgtgcctgtgtgcggctctgatgggaggttttatgaaaaccactgtaagctccaccgtgctgcttgcctcctgggaaagaggatcaccgtcatccacagcaaggactgtttcctcaaaggtgacacgtgcaccatggccggctacgcccgcttgaagaatgtccttctggcactccagacccgtctgcagccactccaagaaggagacagcagacaagaccctgcctcccagaagcgcctcctggtggaatctctgttcagggacttagatgcagatggcaatggccacctcagcagctccgaactggctcagcatgtgctgaagaagcaggacctggatgaagacttacttggttgctcaccaggtgacctcctccgatttgacgattacaacagtgacagctccctgaccctccgcgagttctacatggccttccaagtggttcagctcagcctcgcccccgaggacagggtcagtgtgaccacagtgaccgtggggctgagcacagtgctgacctgcgccgtccatggagacctgaggccaccaatcatctggaagcgcaacgggctcaccctgaacttcctggacttggaagacatcaatgactttggagaggatgattccctgtacatcaccaaggtgaccaccatccacatgggcaattacacctgccatgcttccggccacgagcagctgttccagacccacgtcctgcaggtgaatgtgccgccagtcatccgtgtctatccagagagccaggcacaggagcctggagtggcagccagcctaagatgccatgctgagggcattcccatgcccagaatcacttggctgaaaaacggcgtggatgtctcaactcagatgtccaaacagctctcccttttagccaatgggagcgaactccacatcagcagtgttcggtatgaagacacaggggcatacacctgcattgccaaaaatgaagtgggtgtggatgaagatatctcctcgctcttcattgaagactcagctagaaagacccttgcaaacatcctgtggcgagaggaaggcctcagcgtgggaaacatgttctatgtcttctccgacgacggtatcatcgtcatccatcctgtggactgtgagatccagaggcacctcaaacccacggaaaagattttcatgagctatgaagaaatctgtcctcaaagagaaaaaaatgcaacccagccctgccagtgggtatctgcagtcaatgtccggaaccggtacatctatgtggcccagccagcactgagcagagtccttgtggtcgacatccaagcccagaaagtcctacagtccataggtgtggaccctctgccggctaagctgtcctatgacaagtcacatgaccaagtgtgggtcctgagctggggggacgtgcacaagtcccgaccaagtctccaggtgatcacagaagccagcaccggccagagccagcacctcatccgcacaccctttgcaggagtggatgatttcttcattcccccaacaaacctcatcatcaaccacatcaggtttggcttcatcttcaacaagtctgatcctgcagtccacaaggtggacctggaaacaatgatgcccctcaagaccatcggcctgcaccaccatggctgcgtgccccaggccatggcacacacccacctgggcggctacttcttcatccagtgccgacaggacagccccgcctctgctgcccgacagctgctcgttgacagtgtcacagactctgtgcttggccccaatggtgatgtaacaggcaccccacacacatcccccgacgggcgcttcatagtcagtgctgcagctgacagcccctggctgcacgtgcaggagatcacagtgcggggcgagatccagaccctgtatgacctgcaaataaactcgggcatctcagacttggccttccagcgctccttcactgaaagcaatcaatacaacatctacgcggctctgcacacggagccggacctgctgttcctggagctgtccacggggaaggtgggcatgctgaagaacttaaaggagccacccgcagggccagctcagccctgggggggtacccacagaatcatgagggacagtgggctgtttggacagtacctcctcacaccagcccgagagtcactgttcctcatcaatgggagacaaaacacgctgcggtgtgaggtgtcaggtataaagggggggaccacagtggtgtgggtgggtgaggtactcgag

SEQ ID NO: 87 - protein, 842 aa, Homo sapiens; identical to SEQ ID NO: 16

SEQ ID NO: 88 - protein, 846 aa, Homo sapiens
LEMKPGGFWLHLTLLGASLPAALGWMDPGTSRGPDVGVGESQAEEPRSFEVTRREGLSSHNELLASCGKKFCSRGSRCVLSRKTGEPECQCLEACRPSYVPVCGSDGRFYENHCKLHRAACLLGKRITVIHSKDCFLKGDTCTMAGYARLKNVLLALQTRLQPLQEGDSRQDPASQKRLLVESLFRDLDADGNGHLSSSELAQHVLKKQDLDEDLLGCSPGDLLRFDDYNSDSSLTLREFYMAFQVVQLSLAPEDRVSVTTVTVGLSTVLTCAVHGDLRPPIIWKRNGLTLNFLDLEDINDFGEDDSLYITKVTTIHMGNYTCHASGHEQLFQTHVLQVNVPPVIRVYPESQAQEPGVAASLRCHAEGIPMPRITWLKNGVDVSTQMSKQLSLLANGSELHISSVRYEDTGAYTCIAKNEVGVDEDISSLFIEDSARKTLANILWREEGLSVGNMFYVFSDDGIIVIHPVDCEIQRHLKPTEKIFMSYEEICPQREKNATQPCQWVSAVNVRNRYIYVAQPALSRVLVVDIQAQKVLQSIGVDPLPAKLSYDKSHDQVWVLSWGDVHKSRPSLQVITEASTGQSQHLIRTPFAGVDDFFIPPTNLIINHIRFGFIFNKSDPAVHKVDLETMMPLKTIGLHHHGCVPQAMAHTHLGGYFFIQCRQDSPASAARQLLVDSVTDSVLGPNGDVTGTPHTSPDGRFIVSAAADSPWLHVQEITVRGEIQTLYDLQINSGISDLAFQRSFTESNQYNIYAALHTEPDLLFLELSTGKVGMLKNLKEPPAGPAQPWGGTHRIMRDSGLFGQYLLTPARESLFLINGRQNTLRCEVSGIKGGTTVVWVGEVLE

SEQ ID NO: 89 - DNA, 36 bp, Artificial Sequence; identical to SEQ ID NO: 69
  Description of Artifical Sequence: Primer/Probe

SEQ ID NO: 90 - DNA, 35 bp, Artificial Sequence; identical to SEQ ID NO: 70
  Description of Artifical Sequence: Primer/Probe

SEQ ID NO: 91 - DNA, 36 bp, Artificial Sequence; identical to SEQ ID NO: 69
  Description of Artifical Sequence: Primer/Probe

SEQ ID NO: 92 - DNA, 35 bp, Artificial Sequence; identical to SEQ ID NO: 70
  Description of Artifical Sequence: Primer/Probe

SEQ ID NO: 93 - DNA, 35 bp, Artificial Sequence
  Description of Artifical Sequence: Primer/Probe
ctcgagtggatggacccaggaaccagcagaggccc

SEQ ID NO: 94 - DNA, 35 bp, Artificial Sequence; identical to SEQ ID NO: 70
  Description of Artifical Sequence: Primer/Probe

SEQ ID NO: 95 - DNA, 23 bp, Artificial Sequence
  Description of Artifical Sequence: Primer/Probe
gaaaaccactgtaagctccaccg

SEQ ID NO: 96 - DNA, 27 bp, Artificial Sequence
  Description of Artifical Sequence: Primer/Probe
aggtgacctcctccgatttgacgatta

SEQ ID NO: 97 - DNA, 25 bp, Artificial Sequence
  Description of Artifical Sequence: Primer/Probe
tacatcaccaaggtgaccaccatcc

SEQ ID NO: 98 - DNA, 25 bp, Artificial Sequence
  Description of Artifical Sequence: Primer/Probe
gaagatatctcctcgctcttcattg

SEQ ID NO: 99 - DNA, 25 bp, Artificial Sequence
  Description of Artifical Sequence: Primer/Probe
caagcccagaaagtcctacagtcca

SEQ ID NO: 100 - DNA, 23 bp, Artificial Sequence
  Description of Artifical Sequence: Primer/Probe
acaatgatgcccctcaagaccat

SEQ ID NO: 101 - DNA, 25 bp, Artificial Sequence
  Description of Artifical Sequence: Primer/Probe
caatcaatacaacatctacgcggct

SEQ ID NO: 102 - DNA, 25 bp, Artificial Sequence
  Description of Artifical Sequence: Primer/Probe
tttcataaaacctcccatcagagcc

SEQ ID NO: 103 - DNA, 27 bp, Artificial Sequence
  Description of Artifical Sequence: Primer/Probe
caagtaagtcttcatccaggtcctgct

SEQ ID NO: 104 - DNA, 23 bp, Artificial Sequence
  Description of Artifical Sequence: Primer/Probe
gtgtaattgcccatgtggatggt

SEQ ID NO: 105 - DNA, 25 bp, Artificial Sequence
  Description of Artifical Sequence: Primer/Probe
tatcttcatccacacccacttcatt

SEQ ID NO: 106 - DNA, 23 bp, Artificial Sequence
  Description of Artifical Sequence: Primer/Probe
gatgtcgaccacaaggactctgc

SEQ ID NO: 107 - DNA, 23 bp, Artificial Sequence
  Description of Artifical Sequence: Primer/Probe
ggtcttgaggggcatcattgttt

SEQ ID NO: 108 - DNA, 23 bp, Artificial Sequence
  Description of Artifical Sequence: Primer/Probe
tgattgctttcagtgaaggagcg

SEQ ID NO: 109 - DNA, 23 bp, Artificial Sequence
  Description of Artifical Sequence: Primer/Probe
cacagcaaggactgtttcctcaa

SEQ ID NO: 110 - DNA, 23 bp, Artificial Sequence
  Description of Artifical Sequence: Primer/Probe
ctgaccctccgcgagttctacat

SEQ ID NO: 111 - DNA, 25 bp, Artificial Sequence
  Description of Artifical Sequence: Primer/Probe
gccagtcatccgtgtctatccagag

SEQ ID NO: 112 - DNA, 23 bp, Artificial Sequence
  Description of Artifical Sequence: Primer/Probe
aagacccttgcaaacatcctgtg

SEQ ID NO: 113 - DNA, 22 bp, Artificial Sequence
  Description of Artifical Sequence: Primer/Probe
ctctgccggctaagctgtccta

SEQ ID NO: 114 - DNA, 23 bp, Artificial Sequence
  Description of Artifical Sequence: Primer/Probe
acaatgatgcccctcaagaccat

SEQ ID NO: 115 - DNA, 25 bp, Artificial Sequence
  Description of Artifical Sequence: Primer/Probe
caatcaatacaacatctacgcggct

SEQ ID NO: 116 - DNA, 23 bp, Artificial Sequence
  Description of Artifical Sequence: Primer/Probe
atgacggtgatcctctttcccag

SEQ ID NO: 117 - DNA, 26 bp, Artificial Sequence
  Description of Artifical Sequence: Primer/Probe
agctgtcactgttgtaatcgtcaaat

SEQ ID NO: 118 - DNA, 23 bp, Artificial Sequence
  Description of Artifical Sequence: Primer/Probe
caggtgtaattgcccatgtggat

SEQ ID NO: 119 - DNA, 26 bp, Artificial Sequence
  Description of Artifical Sequence: Primer/Probe
gggtctttctagctgagtcttcaatg

SEQ ID NO: 120 - DNA, 25 bp, Artificial Sequence
  Description of Artifical Sequence: Primer/Probe
tatggactgtaggactttctgggct

SEQ ID NO: 121 - DNA, 23 bp, Artificial Sequence
  Description of Artifical Sequence: Primer/Probe
atggtcttgaggggcatcattgt

SEQ ID NO: 122 - DNA, 23 bp, Artificial Sequence
  Description of Artifical Sequence: Primer/Probe
tgattgctttcagtgaaggagcg

SEQ ID NO: 123 - DNA, 2511 bp, Homo sapiens
  CDS (1)..(2511)
ctcgagatgaaaccaggaggcttttggctgcatctcacactgctcggagcctccctgccggctgcgctgggatggatggacccaggaaccagcagaggcccggatgtgggtgtgggggagtcacaggcagaggagcccagaagctttgaagtcacaagaagagaagggctttccagccacaacgagctgctggcctcctgcgggaagaagttctgcagccgagggagccggtgcgtgctcagcaggaagacaggggagcccgaatgccagtgcctggaggcatgcaggcccagctacgtgcctgtgtgcggctctgatgggaggttttatgaaaaccactgtaagctccaccgtgctgcttgcctcctgggaaagaggatcaccgtcatccacagcaaggactgtttcctcaaaggtgacacgtgcaccattgccggctacgcccgcttgaagaatgtccttctggcactccagacccgtctgcagccactccaagaaggagacagcagacaagaccctgcctcccagaagcgcctcctggtggaatctctgttcagggacttagatgcagatggcaatggccacctcagcagctccgaactggctcagcatgtgctgaagaagcaggacctggatgaagacttacttggttgctcaccaggtgacctcctccgatttgacgattacaacagtgacagctccctgaccctccgcgagttctacatggccttccaagtggttcagctcagcctcgcccccgaggacagggtcagtgtgaccacagtgaccgtggggctgagcacagtgctgacctgcgccgtccatggagacctgaggccaccaatcatctggaagcgcaacgggctcaccctgaacttcctggacttggaagacatcaatgactttggagaggatgattccctgtacatcaccaaggtgaccaccatccacatgggcaattacacctgccatgcttccggccacgagcagctgttccagacccacgtcctgcaggtgaatgtgccgccagtcatccgtgtctatccagagagccaggcacaggagcctggagtggcagccagcctaagatgccatgctgagggcattcccatgcccagaatcacttggctgaaaaacggcgtggatgtctcaactcagatgtccaaacagctctcccttttagccaatgggagcgaactccacatcagcagtgttcggtatgaagacacaggggcatacacctgcattgccaaaaatgaagtgggtgtggatgaagatatctcctcgctcttcattgaagactcagctagaaagacccgcctcagcgtgggaaacatgttctatgtcttctccgacgacggtatcatcgtcatccatcctgtggactgtgagatccagaggcacctcaaacccacggaaaagattttcatgagctatgaagaaatctgtcctcaaagagaaaaaaatgcaacccagccctgccagtgggtatctgcagtcaatgtccggaaccggtacatctatgtggcccagccagcactgagcagagtccttgtggtcgacatccaagcccagaaagtcctacagtccataggtgtggaccctctgccggctaagctgtcctatgacaagtcacatgaccaagtgtgggtcctgagctggggggacgtgcacaagtcccgaccaagtctccaggtgatcacagaagccagcaccggccagagccagcacctcatccgcacaccctttgcaggagtggatgatttcttcattcccccaacaaacctcatcatcaaccacatcaggtttggcttcatcttcaacaagtctgatcctgcagtccacaaggtggacctggaaacaatgatgcccctcaagaccatcggcctgcaccaccatggctgcgtgccccaggccatggcacacacccacctgggcggctacttcttcatccagtgccgacaggacagccccgcctctgctgcccgacagctgctcgttgacagtgtcacagactctgtgcttggccccaatggtgatgtaacaggcaccccacacacatcccccgacgggcgcttcatagtcagtgctgcagctgacagcccctggctgcacgtgcaggagatcacagtgcggggcgagatccagaccctgtatgacctgcaaataaactcgggcatctcagacttggccttccagcgctccttcactgaaagcaatcaatacaacatctacgcggctctgcacatggagccggacctgctgttcctggagctgtccacggggaaggtgggcatgctgaagaacttaaaggagccacccgcagggccagctcagccctgggggggtacccacagaatcatgagggacagtgggctgtttggacagtacctcctcacaccagcccgagagtcactgttcctcatcaatgggagacaaaacacgctgcggtgtgaggtgtcaggtataaagggggggaccacagtggtgtgggtgggtgaggtactcgag

SEQ ID NO: 124 - DNA, 2439 bp, Homo sapiens
  CDS (1)..(2439)
ctcgagatgaaaccaggaggcttttggctgcatctcacactgctcggagcctccctgccggctgcgctgggatggatggacccaggaaccagcagaggcccggatgtgggtgtgggggagtcacaggcagaggagcccagaagctttgaagtcacaagaagagaagggctttccagccacaacgagctgctggcctcctgcgggaagaagttctgcagccgagggagccggtgcgtgctcagcaggaagacaggggagcccgaatgcctgggaaagaggatcaccgtcatccacagcaaggactgtttcctcaaaggtgacacgtgcaccatggccggctacgcccgcttgaagaatgtccttctggcactccagacccgtctgcagccactccaagaaggagacagcagacaagaccctgcctcccagaagcgcctcctggtggaatctctgttcagggacttagatgcagatggcaatggccacctcagcagctccgaactggctcagcatgtgctgaagaagcaggacctggatgaagacttacttggttgctcaccaggtgacctcctccgatttgacgattacaacagtgacagctccctgaccctccgcgagttctacatggccttccaagtggttcagctcagcctcgcccccgaggacagggtcagtgtgaccacagtgaccgtggggctgagcacagtgctgacctgcgccgtccatggagacctgaggccaccaatcatctggaagcgcaacgggctcaccctgaacttcctggacttggaagacatcaatgactttggagaggatgattccctgtacatcaccaaggtgaccaccatccacatgggcaattacacctgccatgcttccggccacgagcagctgttccagacccacgtcctgcaggtgaatgtgccgccagtcatccgtgtctatccagagagccaggcacaggagcctggagtggcagccagcctaagatgccatgctgagggcattcccatgcccagaatcacttggctgaaaaacggcgtggatgtctcaactcagatgtccaaacagctctcccttttagccaatgggagcgaactccacatcagcagtgttcggtatgaagacacaggggcatacacctgcattgccaaaaatgaagtgggtgtggatgaagatatctcctcgctcttcattgaagactcagctagaaagacccttgcaaacatcctgtggcgagaggaaggcctcagcgtgggaaacatgttctatgtcttctccgacgacggtatcatcgtcatccatcctgtggactgtgagatccagaggcacctcaaacccacggaaaagattttcatgagctatgaagaaatctgtcctcaaagagaaaaaaatgcaacccagccctgccagtgggtatctgcagtcaatgtccggaaccggtacatctatgtggcccagccagcactgagcagagtccttgtggtcgacatccaagcccagaaagtcctacagtccataggtgtggaccctctgccggctaagctgtcctatgacaagtcacatgaccaagtgtgggtcctgagctggggggacgtgcacaagtcccgaccaagtctccaggtgatcacagaagccagcaccggccagagccagcacctcatccgcacaccctttgcaggagtggatgatttcttcattcccccaacaaacctcatcatcaaccacatcaggtttggcttcatcttcaacaagtctgatcctgcagtccacaaggtggacctggaaacaatgatgcccctcaagaccatcggcctgcaccaccatggctgcgtgccccaggccatggcacacacccacctgggcggctacttcttcatccagtgccgacaggacagccccgcctctgctgcccgacagctgctcgttgacagtgtcacagactctgtgcttggccccaatggtgatgtaacaggcaccccacacacatcccccgacgggcgcttcatagtcagtgctgcagctgacagcccctggctgcacgtgcaggagatcacagtgcggggcgagatccagaccctgtatgacctgcaaataaactcgggcatctcagacttggccttccagcgctccttcactgaaagcaatcaatacaacatctacgcggctctgcacatggagccggacctgctgttcctggagctgtccacggggaaggtgggcatgctgaagaacttaaaggagccacccgcagggccagctcagccctgggggggtacccacagaatcatgagggacagtgggctgtttggacagtacctcctcacaccagcccgagagtcactgttcctcatcaatgggagacaaaacacgctgcggtgtgaggtgtcaggtataaagggggggaccacagtggtgtgggtgggtgaggtactcgag

SEQ ID NO: 125 - DNA, 2472 bp, Homo sapiens
ctcgagtacctcacccacccacaccactgtggtccccccctttatacctgacacctcacaccgcagcgtgttttgtctcccattgatgaggaacagtgactctcgggctggtgtgaggaggtactgtccaaacagcccactgtccctcatgattctgtgggtacccccccagggctgagctggccctgcgggtggctcctttaagttcttcagcatgcccaccttccccgtggacagctccaggaacagcaggtccggctccgtgtgcagagccgcgtagatgttgtattgattgctttcagtgaaggagcgctggaaggccaagtctgagatgcccgagtttatttgcaggtcatacagggtctggatctcgccccgcactgtgatctcctgcacgtgcagccaggggctgtcagctgcagcactgactatgaagcgcccgtcgggggatgtgtgtggggtgcctgttacatcaccattggggccaagcacagagtctgtgacactgtcaacgagcagctgtcgggcagcagaggcggggctgtcctgtcggcactggatgaagaagtagccgcccaggtgggtgtgtgccatggcctggggcacgcagccatggtggtgcaggccgatggtcttgaggggcatcattgtttccaggtccaccttgtggactgtaggatcagacttgttgaagatgaagccaaacctgatgtggttgatgatgaggtttgttgggggaatgaagaaatcatccactcctgcaaagggtgtgcggatgaggtgctggctctggccggtgctggcttctgtgatcacctggagacttggtcgggacttgtgcacgtccccccagctcaggacccacacttggtcatgtgacttgtcataggacagcttagccggcagagggtccacacctatggactgtaggactttctgggcttggatgtcgaccacaaggactctgctcagtgctggctgggccacatagatgtaccggttccggacattgactgcagatacccactggcagggctgggttgcatttttttctctttgaggacagatttcttcatagctcatgaaaatcttttccgtgggtttgaggtgcctctggatctcacagtccacaggatggatgacgatgataccgtcgtcggagaagacatagaacatgtttcccacgctgaggccttcctctcgccacaggatgtttgcaagggtctttctagctgagtcttcaatgaagagcgaggagatatcttcatccacacccacttcatttttggcaatgcaggtgtatgcccctgtgtcttcataccgaacactgctgatgtggagttcgctcccattggctaaaagggagagctgtttggacatctgagttgagacatccacgccgtttttcagccaagtgattctgggcatgggaatgccctcagcatggcatcttaggctggctgccactccaggctcctgtgcctggctctctggatagacacggatgactggcggcacattcacctgcaggacgtgggtctggaacagctgctcgtggccggaagcatggcaggtgtaattgcccatgtggatggtggtcaccttggtgatgtacagggaatcatcctctccaaagtcattgatgtcttccaagtccaggaagttcagggtgagcccgttgcgcttccagatgattggtggcctcaggtctccatggacggcgcaggtcagcactgtgctcagccccacggtcactgtggtcacactgaccctgtcctcgggggcgaggctgagctgaaccacttggaaggccatgtagaactcgcggagggtcagggagctgtcactgttgtaatcgtcaaatcggaggaggtcacctggtgagcaaccaagtaagtcttcatccaggtcctgcttcttcagcacatgctgagccagttcggagctgctgaggtggccattgccatctgcatctaagtccctgaacagagattccaccaggaggcgcttctgggaggcagggtcttgtctgctgtctccttcttggagtggctgcagacgggtctggagtgccagaaggacattcttcaagcgggcgtagccggccatggtgcacgtgtcacctttgaggaaacagtccttgctgtggatgacggtgatcctctttcccaggaggcaagcagcacggtggagcttacagtggttttcataaaacctcccatcagagccgcacacaggcacgtagctgggcctgcatgcctccaggcactggcattcgggctcccctgtcttcctgctgagcacgcaccggctccctcggctgcagaacttcttcccgcaggaggccagcagctcgttgtggctggaaagcccttctcttcttgtgacttcaaagcttctgggctcctctgcctgtgactcccccacacccacatccgggcctctgctggttcctgggtccatccactcgag

SEQ ID NO: 126 - protein, 837 aa, Homo sapiens
LEMKPGGFWLHLTLLGASLPAALGWMDPGTSRGPDVGVGESQAEEPRSFEVTRREGLSSHNELLASCGKKFCSRGSRCVLSRKTGEPECQCLEACRPSYVPVCGSDGRFYENHCKLHRAACLLGKRITVIHSKDCFLKGDTCTIAGYARLKNVLLALQTRLQPLQEGDSRQDPASQKRLLVESLFRDLDADGNGHLSSSELAQHVLKKQDLDEDLLGCSPGDLLRFDDYNSDSSLTLREFYMAFQVVQLSLAPEDRVSVTTVTVGLSTVLTCAVHGDLRPPIIWKRNGLTLNFLDLEDINDFGEDDSLYITKVTTIHMGNYTCHASGHEQLFQTHVLQVNVPPVIRVYPESQAQEPGVAASLRCHAEGIPMPRITWLKNGVDVSTQMSKQLSLLANGSELHISSVRYEDTGAYTCIAKNEVGVDEDISSLFIEDSARKTRLSVGNMFYVFSDDGIIVIHPVDCEIQRHLKPTEKIFMSYEEICPQREKNATQPCQWVSAVNVRNRYIYVAQPALSRVLVVDIQAQKVLQSIGVDPLPAKLSYDKSHDQVWVLSWGDVHKSRPSLQVITEASTGQSQHLIRTPFAGVDDFFIPPTNLIINHIRFGFIFNKSDPAVHKVDLETMMPLKTIGLHHHGCVPQAMAHTHLGGYFFIQCRQDSPASAARQLLVDSVTDSVLGPNGDVTGTPHTSPDGRFIVSAAADSPWLHVQEITVRGEIQTLYDLQINSGISDLAFQRSFTESNQYNIYAALHMEPDLLFLELSTGKVGMLKNLKEPPAGPAQPWGGTHRIMRDSGLFGQYLLTPARESLFLINGRQNTLRCEVSGIKGGTTVVWVGEVLE

SEQ ID NO: 127 - protein, 813 aa, Homo sapiens
LEMKPGGFWLHLTLLGASLPAALGWMDPGTSRGPDVGVGESQAEEPRSFEVTRREGLSSHNELLASCGKKFCSRGSRCVLSRKTGEPECLGKRITVIHSKDCFLKGDTCTMAGYARLKNVLLALQTRLQPLQEGDSRQDPASQKRLLVESLFRDLDADGNGHLSSSELAQHVLKKQDLDEDLLGCSPGDLLRFDDYNSDSSLTLREFYMAFQVVQLSLAPEDRVSVTTVTVGLSTVLTCAVHGDLRPPIIWKRNGLTLNFLDLEDINDFGEDDSLYITKVTTIHMGNYTCHASGHEQLFQTHVLQVNVPPVIRVYPESQAQEPGVAASLRCHAEGIPMPRITWLKNGVDVSTQMSKQLSLLANGSELHISSVRYEDTGAYTCIAKNEVGVDEDISSLFIEDSARKTLANILWREEGLSVGNMFYVFSDDGIIVIHPVDCEIQRHLKPTEKIFMSYEEICPQREKNATQPCQWVSAVNVRNRYIYVAQPALSRVLVVDIQAQKVLQSIGVDPLPAKLSYDKSHDQVWVLSWGDVHKSRPSLQVITEASTGQSQHLIRTPFAGVDDFFIPPTNLIINHIRFGFIFNKSDPAVHKVDLETMMPLKTIGLHHHGCVPQAMAHTHLGGYFFIQCRQDSPASAARQLLVDSVTDSVLGPNGDVTGTPHTSPDGRFIVSAAADSPWLHVQEITVRGEIQTLYDLQINSGISDLAFQRSFTESNQYNIYAALHMEPDLLFLELSTGKVGMLKNLKEPPAGPAQPWGGTHRIMRDSGLFGQYLLTPARESLFLINGRQNTLRCEVSGIKGGTTVVWVGEVLE

SEQ ID NO: 128 - protein, 824 aa, Homo sapiens
LEWMDPGTSRGPDVGVGESQAEEPRSFEVTRREGLSSHNELLASCGKKFCSRGSRCVLSRKTGEPECQCLEACRPSYVPVCGSDGRFYENHCKLHRAACLLGKRITVIHSKDCFLKGDTCTMAGYARLKNVLLALQTRLQPLQEGDSRQDPASQKRLLVESLFRDLDADGNGHLSSSELAQHVLKKQDLDEDLLGCSPGDLLRFDDYNSDSSLTLREFYMAFQVVQLSLAPEDRVSVTTVTVGLSTVLTCAVHGDLRPPIIWKRNGLTLNFLDLEDINDFGEDDSLYITKVTTIHMGNYTCHASGHEQLFQTHVLQVNVPPVIRVYPESQAQEPGVAASLRCHAEGIPMPRITWLKNGVDVSTQMSKQLSLLANGSELHISSVRYEDTGAYTCIAKNEVGVDEDISSLFIEDSARKTLANILWREEGLSVGNMFYVFSDDGIIVIHPVDCEIQRHLKPTEKIFMSYEEICPQREKNATQPCQWVSAVNVRNRYIYVAQPALSRVLVVDIQAQKVLQSIGVDPLPAKLSYDKSHDQVWVLSWGDVHKSRPSLQVITEASTGQSQHLIRTPFAGVDDFFIPPTNLIINHIRFGFIFNKSDPTVHKVDLETMMPLKTIGLHHHGCVPQAMAHTHLGGYFFIQCRQDSPASAARQLLVDSVTDSVLGPNGDVTGTPHTSPDGRFIVSAAADSPWLHVQEITVRGEIQTLYDLQINSGISDLAFQRSFTESNQYNIYAALHTEPDLLFLELSTGKVGMLKNLKEPPAGPAQPWGGTHRIMRDSGLFGQYLLTPARESLFLINGRQNTLRCEVSGIKGGTTVVWVGEVLE

SEQ ID NO: 129 - DNA, 36 bp, Artificial Sequence; identical to SEQ ID NO: 69
  Description of Artifical Sequence: Primer/Probe

SEQ ID NO: 130 - DNA, 35 bp, Artificial Sequence; identical to SEQ ID NO: 70
  Description of Artifical Sequence: Primer/Probe

SEQ ID NO: 131 - DNA, 23 bp, Artificial Sequence
  Description of Artifical Sequence: Primer/Probe
gaaaaccactgtaagctccaccg

SEQ ID NO: 132 - DNA, 27 bp, Artificial Sequence
  Description of Artifical Sequence: Primer/Probe
aggtgacctcctccgatttgacgatta

SEQ ID NO: 133 - DNA, 25 bp, Artificial Sequence
  Description of Artifical Sequence: Primer/Probe
tacatcaccaaggtgaccaccatcc

SEQ ID NO: 134 - DNA, 25 bp, Artificial Sequence
  Description of Artifical Sequence: Primer/Probe
gaagatatctcctcgctcttcattg

SEQ ID NO: 135 - DNA, 25 bp, Artificial Sequence
  Description of Artifical Sequence: Primer/Probe
caagcccagaaagtcctacagtcca

SEQ ID NO: 136 - DNA, 23 bp, Artificial Sequence
  Description of Artifical Sequence: Primer/Probe
acaatgatgcccctcaagaccat

SEQ ID NO: 137 - DNA, 25 bp, Artificial Sequence
  Description of Artifical Sequence: Primer/Probe
caatcaatacaacatctacgcggct

SEQ ID NO: 138 - DNA, 25 bp, Artificial Sequence
  Description of Artifical Sequence: Primer/Probe
tttcataaaacctcccatcagagcc

SEQ ID NO: 139 - DNA, 27 bp, Artificial Sequence
  Description of Artifical Sequence: Primer/Probe
caagtaagtcttcatccaggtcctgct

SEQ ID NO: 140 - DNA, 23 bp, Artificial Sequence
  Description of Artifical Sequence: Primer/Probe
gtgtaattgcccatgtggatggt

SEQ ID NO: 141 - DNA, 25 bp, Artificial Sequence
  Description of Artifical Sequence: Primer/Probe
tatcttcatccacacccacttcatt

SEQ ID NO: 142 - DNA, 23 bp, Artificial Sequence
  Description of Artifical Sequence: Primer/Probe
gatgtcgaccacaaggactctgc

SEQ ID NO: 143 - DNA, 23 bp, Artificial Sequence
  Description of Artifical Sequence: Primer/Probe
ggtcttgaggggcatcattgttt

SEQ ID NO: 144 - DNA, 375 bp, Homo sapiens
  CDS (1)..(375)
aagctttcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccaggtgagtggacatttgcatgccatctttatgaactcgag

SEQ ID NO: 145 - protein, 125 aa, Homo sapiens
KLSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPGEWTFACHLYELE

SEQ ID NO: 146 - protein, 142 aa, Homo sapiens; identical to SEQ ID NO: 22

SEQ ID NO: 147 - DNA, 2538 bp, Homo sapiens
  CDS (1)..(2538)
ctcgagatgaaaccaggaggcttttggctgcatctcacactgctcggagcctccctgccggctgcgctgggatggatggacccaggaaccagcagaggcccggatgtgggtgtgggggagtcacaggcagaggagcccagaagctttgaagtcacaagaagagaagggctttccagccacaacgagctgctggcctcctgcgggaagaagttctgcagccgagggagccggtgcgtgctcagcaggaagacaggggagcccgaatgccagtgcctggaggcatgcaggcccagctacgtgcctgtgtgcggctctgatgggaggttttatgaaaaccactgtaagctccaccgtgctgcttgcctcctgggaaagaggatcaccgtcatccacagcaaggactgtttcctcaaaggtgacacgtgcaccatggccggctacgcccgcttgaagaatgtccttctggcactccagacccgtctgcagccactccaagaaggagacagcagacaagaccctgcctcccagaagcgcctcctggtggaatctctgttcagggacttagatgcagatggcaatggccacctcagcagctccgaactggctcagcatgtgctgaagaagcaggacctggatgaagacttacttggttgctcaccaggtgacctcctccgatttgacgattacaacagtgacagctccctgaccctccgcgagttctacatggccttccaagtggttcagctcagcctcgcccccgaggacagggtcagtgtgaccacagtgaccgtggggctgagcacagtgctgacctgcgccgtccatggagacctgaggccaccaatcatctggaagcgcaacgggctcaccctgaacttcctggacttggaagacatcaatgactttggagaggatgattccctgtacatcaccaaggtgaccaccatccacatgggcaattacacctgccatgcttccggccacgagcagctgttccagacccacgtcctgcaggtgaatgtgccgccagtcatccgtgtctatccagagagccaggcacaggagcctggagtggcagccagcctaagatgccatgctgagggcattcccatgcccagaatcacttggctgaaaaacggcgtggatgtctcaactcagatgtccaaacagctctcccttttagccaatgggagcgaactccacatcagcagtgttcggtatgaagacacaggggcatacacctgcattgccaaaaatgaagtgggtgtggatgaagatatctcctcgctcttcattgaagactcagctagaaagacccttgcaaacatcctgtggcgagaggaaggcctcagcgtgggaaacatgttctatgtcttctccgacgacggtatcatcgtcatccatcctgtggactgtgagatccagaggcacctcaaacccacggaaaagattttcatgagctatgaagaaatctgtcctcaaagagaaaaaaatgcaacccagccctgccagtgggtatctgcagtcaatgtccggaaccggtacatctatgtggcccagccagcactgagcagagtccttgtggtcgacatccaagcccagaaagtcctacagtccataggtgtggaccctctgccggctaagctgtcctatgacaagtcacatgaccaagtgtgggtcctgagctggggggacgtgcacaagtcccgaccaagtctccaggtgatcacagaagccagcaccggccagagccagcacctcatccgcacaccctttgcaggagtggatgatttcttcattcccccaacaaacctcatcatcaaccacatcaggtttggcttcatcttcaacaagtctgatcctgcagtccacaaggtggacctggaaacaatgatgcccctcaagaccatcggcctgcaccaccatggctgcgtgccccaggccatggcacacacccacctgggcggctacttcttcatccagtgccgacaggacagccccgcctctgctgcccgacagctgctcgttgacagtgtcacagactctgtgcttggccccaatggtgatgtaacaggcaccccacacacatcccccgacgggcgcttcatagtcagtgctgcagctgacagcccctggctgcacgtgcaggagatcacagtgcggggcgagatccagaccctgtatgacctgcaaataaactcgggcatctcagacttggccttccagcgctccttcactgaaagcaatcaatacaacatctacgcggctctgcacatggagccggacctgctgttcctggagctgtccacggggaaggtgggcatgctgaagaacttaaaggagccacccgcagggccagctcagccctgggggggtacccacagaatcatgagggacagtgggctgtttggacagtacctcctcacaccagcccgagagtcactgttcctcatcaatgggagacaaaacacgctgcggtgtgaggtgtcaggtataaagggggggaccacagtggtgtgggtgggtgaggtactcgag

SEQ ID NO: 148 - protein, 846 aa, Homo sapiens
LEMKPGGFWLHLTLLGASLPAALGWMDPGTSRGPDVGVGESQAEEPRSFEVTRREGLSSHNELLASCGKKFCSRGSRCVLSRKTGEPECQCLEACRPSYVPVCGSDGRFYENHCKLHRAACLLGKRITVIHSKDCFLKGDTCTMAGYARLKNVLLALQTRLQPLQEGDSRQDPASQKRLLVESLFRDLDADGNGHLSSSELAQHVLKKQDLDEDLLGCSPGDLLRFDDYNSDSSLTLREFYMAFQVVQLSLAPEDRVSVTTVTVGLSTVLTCAVHGDLRPPIIWKRNGLTLNFLDLEDINDFGEDDSLYITKVTTIHMGNYTCHASGHEQLFQTHVLQVNVPPVIRVYPESQAQEPGVAASLRCHAEGIPMPRITWLKNGVDVSTQMSKQLSLLANGSELHISSVRYEDTGAYTCIAKNEVGVDEDISSLFIEDSARKTLANILWREEGLSVGNMFYVFSDDGIIVIHPVDCEIQRHLKPTEKIFMSYEEICPQREKNATQPCQWVSAVNVRNRYIYVAQPALSRVLVVDIQAQKVLQSIGVDPLPAKLSYDKSHDQVWVLSWGDVHKSRPSLQVITEASTGQSQHLIRTPFAGVDDFFIPPTNLIINHIRFGFIFNKSDPAVHKVDLETMMPLKTIGLHHHGCVPQAMAHTHLGGYFFIQCRQDSPASAARQLLVDSVTDSVLGPNGDVTGTPHTSPDGRFIVSAAADSPWLHVQEITVRGEIQTLYDLQINSGISDLAFQRSFTESNQYNIYAALHMEPDLLFLELSTGKVGMLKNLKEPPAGPAQPWGGTHRIMRDSGLFGQYLLTPARESLFLINGRQNTLRCEVSGIKGGTTVVWVGEVLE

SEQ ID NO: 149 - DNA, 1037 bp, Homo sapiens
gcgagaaggcgatgtgtgacttcaccggcccagggagcttcgtgtcagggatggcccggcccctccctactggagagtgggtccgccaggcacaggtgggtggagaacattcacgaaagagtgaggtggggtcggggatggggcgacagggaccagcagggccaaggacaaggtgcaccagtctagcggtcctcccggaccggccggatcttcatctcagagaacttgagtgagtactgccagccggtccaggaggaccactccacgccgtcggcataggaggcgtgcgcaccgcgcaggtactgcccattgaggttggacgtgtggcagttgcggtaccaccaggcaccgcggtagaaggcggcacagttgttctctgaatggtcgctgtcacggtccttggtggtgaacctcatgccgctgtgcttcaggagggagtcgcctgcagtgccggaatagtcagccacggtgagcgggtacccgtcttcctcagggtccacggagaacaagcccacgccgaagctcccgtagcgggcataggccgtgccattctcaaagtcctccaggtccacgtgcagctcgtaggcagcctgtgtggtcagggcgtggatcctcttgagccctagccagtgctccccggtgagcctgccaaagccgtctcggtacgcatcccagccccggaagaagttcacggagccgtcctcccggcgctgaaacaccgtccagccgccgccgtccgtgcgcatgtcacagtacacctggaagccggccgggtagtgggtgggaaagacagagtagacgccatcgtcctgctgtccgcttaggaggacgtccagacagtctcggggccgggagccagtggcacagccccggggccgggttccccgggcaggcgctctctgaaggtcggccttgttgcggggccggcccagcccccggtccctctgcagggcatccaggatgtcgctgacggagttcaccaggtgagccatgtgggcctggctctcagagagaagctggatgaggcggcccttgct

SEQ ID NO: 150 - protein, 269 aa, Homo sapiens; identical to SEQ ID NO: 26

SEQ ID NO: 151 - DNA, 23 bp, Artificial Sequence
  Description of Artifical Sequence: Primer/Probe
tgattgctttcagtgaaggagcg

SEQ ID NO: 152 - DNA, 2472 bp, Homo sapiens
  CDS (1)..(2472)
ctcgagtggatggacccaggaaccagcagaggcccggatgtgggtgtgggggagtcacaggcagaggagcccagaagctttgaagtcacaagaagagaagggctttccagccacaacgagctgctggcctcctgcgggaagaagttctgcagccgagggagccggtgcgtgctcagcaggaagacaggggagcccgaatgccagtgcctggaggcatgcaggcccagctacgtgcctgtgtgcggctctgatgggaggttttatgaaaaccactgtaagctccaccgtgctgcttgcctcctgggaaagaggatcaccgtcatccacagcaaggactgtttcctcaaaggtgacacgtgcaccatggccggctacgcccgcttgaagaatgtccttctggcactccagacccgtctgcagccactccaagaaggagacagcagacaagaccctgcctcccagaagcgcctcctggtggaatctctgttcagggacttagatgcagatggcaatggccacctcagcagctccgaactggctcagcatgtgctgaagaagcaggacctggatgaagacttacttggttgctcaccaggtgacctcctccgatttgacgattacaacagtgacagctccctgaccctccgcgagttctacatggccttccaagtggttcagctcagcctcgcccccgaggacagggtcagtgtgaccacagtgaccgtggggctgagcacagtgctgacctgcgccgtccatggagacctgaggccaccaatcatctggaagcgcaacgggctcaccctgaacttcctggacttggaagacatcaatgactttggagaggatgattccctgtacatcaccaaggtgaccaccatccacatgggcaattacacctgccatgcttccggccacgagcagctgttccagacccacgtcctgcaggtgaatgtgccgccagtcatccgtgtctatccagagagccaggcacaggagcctggagtggcagccagcctaagatgccatgctgagggcattcccatgcccagaatcacttggctgaaaaacggcgtggatgtctcaactcagatgtccaaacagctctcccttttagccaatgggagcgaactccacatcagcagtgttcggtatgaagacacaggggcatacacctgcattgccaaaaatgaagtgggtgtggatgaagatatctcctcgctcttcattgaagactcagctagaaagacccttgcaaacatcctgtggcgagaggaaggcctcagcgtgggaaacatgttctatgtcttctccgacgacggtatcatcgtcatccatcctgtggactgtgagatccagaggcacctcaaacccacggaaaagattttcatgagctatgaagaaatctgtcctcaaagagaaaaaaatgcaacccagccctgccagtgggtatctgcagtcaatgtccggaaccggtacatctatgtggcccagccagcactgagcagagtccttgtggtcgacatccaagcccagaaagtcctacagtccataggtgtggaccctctgccggctaagctgtcctatgacaagtcacatgaccaagtgtgggtcctgagctggggggacgtgcacaagtcccgaccaagtctccaggtgatcacagaagccagcaccggccagagccagcacctcatccgcacaccctttgcaggagtggatgatttcttcattcccccaacaaacctcatcatcaaccacatcaggtttggcttcatcttcaacaagtctgatcctacagtccacaaggtggacctggaaacaatgatgcccctcaagaccatcggcctgcaccaccatggctgcgtgccccaggccatggcacacacccacctgggcggctacttcttcatccagtgccgacaggacagccccgcctctgctgcccgacagctgctcgttgacagtgtcacagactctgtgcttggccccaatggtgatgtaacaggcaccccacacacatcccccgacgggcgcttcatagtcagtgctgcagctgacagcccctggctgcacgtgcaggagatcacagtgcggggcgagatccagaccctgtatgacctgcaaataaactcgggcatctcagacttggccttccagcgctccttcactgaaagcaatcaatacaacatctacgcggctctgcacacggagccggacctgctgttcctggagctgtccacggggaaggtgggcatgctgaagaacttaaaggagccacccgcagggccagctcagccctgggggggtacccacagaatcatgagggacagtgggctgtttggacagtacctcctcacaccagcccgagagtcactgttcctcatcaatgggagacaaaacacgctgcggtgtgaggtgtcaggtataaagggggggaccacagtggtgtgggtgggtgaggtactcgag

(19) United States
(12) Patent Application Publication
(10) Pub. No.: US 2005/0170380 A1
(43) Pub. Date: Aug. 4, 2005

(54) Novel human proteins and polynucleotides encoding them

(75) Inventors: Elma R. Fernandes, Branford, CT (US); Corine A.M. Vernet, Branford, CT (US); Richard A. Shimkets, Guilford, CT (US); David W. Anderson, Branford, CT (US); Muralidhara Padigaru, Branford, CT (US); Ferenc L. Boldog, North Haven, CT (US); Li Li, Branford, CT (US); Suresh G. Shenoy, Branford, CT (US); Stacie J. Casman, North Haven, CT (US); Luca Rastelli, Guilford, CT (US)

Correspondence Address: GEORGE YAHWAK, ESQ., 555 LONG WHARF DRIVE, 9TH FLOOR, NEW HAVEN, CT, 06511, US

(73) Assignee: CuraGen Corporation, New Haven, CT (US)

(21) Appl. No.: 10/971,479

(22) Filed: Oct. 21, 2004

Related U.S. Application Data
(63) Continuation of application No. 10/189,940, filed on Jul. 3, 2002, now abandoned.
(60) Provisional application No. 60/303,241, filed on Jul. 5, 2001.
(60) Provisional application No. 60/369,065, filed on Apr. 1, 2002.
(60) Provisional application No. 60/378,730, filed on May 8, 2002.
(60) Provisional application No. 60/384,327, filed on May 30, 2002.

(51) Int. Cl. C12Q 1/68; C07H 21/04; C07K 14/47; C07K 16/18

(52) U.S. Cl. 435/6; 435/69.1; 435/320.1; 435/325; 530/350; 530/388.1; 536/23.2

(57) ABSTRACT

The present invention provides novel isolated SECX polynucleotides and the membrane-associated or secreted polypeptides encoded by the SECX polynucleotides. Also provided are the antibodies that immunospecifically bind to a SECX polypeptide or any derivative, variant, mutant or fragment of the SECX polypeptide, polynucleotide or antibody. The invention additionally provides methods in which the SECX polypeptide, polynucleotide and antibody are utilized in the detection and treatment of a broad range of pathological states, as well as to other uses.

### RELATED APPLICATIONS

This application claims priority to U.S. provisional patent applications Ser. Nos. 60/303,241 filed Jul. 5, 2001, 60/369,065 filed Apr. 1, 2002, 60/378,730 filed May 8, 2002, and 60/384,327 filed 30, 2002, and U.S. utility patent applications Ser No. 09/965,212 filed Sep. 26, 2001, Ser. No. 09/966,545 filed Sep. 26, 2001 and Ser. No. 09/966,546 filed Sep. 26, 2001, all claiming priority to U.S. utility patent application Ser. No. 09/544,511 filed Apr. 6, 2000, which in turn claims priority to U.S. provisional applications Ser. No. 60/128,514, filed Apr. 9, 1999 and Ser. No.60/186,592, filed Mar. 3, 2000, each incorporated herein by reference in their entirety.

### FIELD OF THE INVENTION

The invention relates to polynucleotides and polypeptides encoded by such polynucleotides, as well as vectors, host cells, antibodies and recombinant methods for producing the polypeptides and polynucleotides.

### BACKGROUND OF THE INVENTION

Eukaryotic cells are subdivided by membranes into multiple functionally distinct compartments that are referred to as organelles. Each organelle includes proteins essential for-its proper function. These proteins can include sequence motifs often referred to as sorting signals. The sorting signals can aid in targeting the proteins to their appropriate cellular organelle. In addition, sorting signals can direct some proteins to be exported, or secreted, from the cell.

One type of sorting signal is a signal sequence, which is also referred to as a signal peptide or leader sequence. The signal sequence is present as an amino-terminal extension on a newly synthesized polypeptide chain A signal sequence can target proteins to an intracellular organelle called the endoplasmic reticulum (ER).

The signal sequence takes part in an array of protein-protein and protein-lipid interactions that result in translocation of a polypeptide containing the signal sequence through a channel in the ER. After translocation, a membrane-bound enzyme, named a signal peptidase, liberates the mature protein from the signal sequence.

The ER functions to separate membrane-bound proteins and secreted proteins from proteins that remain in the cytoplasm. Once targeted to the ER, both secreted and membrane-bound proteins can be further distributed to another cellular organelle called the Golgi apparatus. The Golgi directs the proteins to other cellular organelles such as vesicles, lysosomes, the plasma membrane, mitochondria and microbodies.

Only a limited number of genes encoding human membrane-bound and secreted proteins have been identified. Examples of known secreted proteins include human insulin, interferon, interleukins, transforming growth factor-beta, human growth hormone, erythropoietin, and lymphokines.

### SUMMARY OF THE INVENTION

The present invention is based, in part, upon the discovery of novel human polynucleotide sequences and polypeptides encoded by these sequences. Polypeptides or synonymously proteins of the invention include an IL-17-like protein (clone 2191999), putative cell adhesion protein variants (clones 11753149.0.6 and 11753149.0.37), a putative surface membrane associated protein (clone 3883556 and the cDNA clone pCDNA3.1-TOPO-3883556-S54), PCK-1-like protein variants (clones 4301136-1 and 4301136-2), surface adhesion protein-like variants (clones 4324229 and 4324229-2), surface adhesion protein-like protein (AC012614_{—}1.0.123), mitochondrial membrane- or plasma membrane-associated protein variants (clones 4339264-2 and 4339264-3), a putative microbody (peroxisome) associated protein (clone 4391184), and an opsonin-like and/or MAG4V-like protein and its cDNA variants (clones 4437909.0.4, 4437909.0.55 and cDNA TA-4437909-S443), follistatin-like proteins (CG52643-02, AC012614_{—}1.0.123 and 4324229-2). Proteins of the invention include both the full length protein encoded by the open reading frame of the nucleic acid herein, as well as the processed mature form of the protein. Both the precursor and the mature forms of the proteins of the invention are described herein. These polynucleotides and the polypeptides encoded thereby are collectively referred to as the SECX gene set, the sequences of which are disclosed in SEQ ID NOs:1-30, 85-88, 123-127, 144-150.

In one aspect, the invention includes an isolated SECX nucleic acid molecule which includes a nucleotide sequence encoding a polypeptide that includes the amino acid sequence of one or more of SEQ ID NOs:2, 4, 6, 8, 10, 12, 14, 16, 18, 20, 22, 24, 26, 28, 30, 87, 88, 126, 127, 145, 146, 148 and 150. For example, in various embodiments, the nucleic acid can include a nucleotide sequence that includes SEQ ID NOs:1, 3, 5, 7, 9, 11, 13, 15, 17, 19, 21, 23, 25, 27, 29, 85, 86, 123-125, 144, 147 and 149. Alternatively, the encoded SECX polypeptide may have a variant amino acid sequence, e.g., have an identity or similarity less than 100% to the disclosed amino acid sequences, as described herein.

The invention also includes an isolated polypeptide that includes the amino acid sequence of one or more of SEQ ID NOs:2, 4, 6, 8, 10, 12, 14, 16, 18, 20, 22, 24, 26, 28, 30, 87, 88, 126, 127, 145, 146, 148, or 150 or a fragment having at least 15 amino acids of these amino acid sequences. Also included is a naturally occurring polypeptide variant of a SECX polypeptide, wherein the polypeptide is encoded by a nucleic acid molecule which hybridizes under stringent conditions to a nucleic acid molecule consisting of a SECX nucleic acid molecule.

Also included in the invention is an antibody which selectively binds to a SECX polypeptide.

The invention further includes a method for producing a SECX polypeptide by culturing a host cell expressing one of the herein described SECX nucleic acids under conditions in which the nucleic acid molecule is expressed.

The invention also includes methods for detecting the presence and amount of a SECX polypeptide or nucleic acid in a sample from a mammal, e.g., a human, by contacting a sample from the mammal with an antibody which selectively binds to one of the herein described polypeptides, and detecting the formation of reaction complexes including the antibody and the polypeptide in the sample. Detecting the formation of complexes in the sample indicates the presence of the polypeptide in the sample. Methods for measurements of antibody reaction complex concentrations are well known in the art. Methods for detecting and quantitating nucleic acids include hybridization and TaqMan™ quantitation.

The invention further includes a method for detecting or diagnosing the presence of a disease, e.g., a pathological condition, associated with altered levels of a polypeptide having an amino acid sequence at least 80% identical to a SECX polypeptide in a sample. The method includes measuring the level of the polypeptide in a biological sample from the mammalian subject, e.g., a human, and comparing the level detected to a level of the polypeptide present in normal subjects, or in the same subject at a different time, e.g., prior to onset of a condition. An increase or decrease in the level of the polypeptide as compared to normal levels indicates a disease condition.

Also included in the invention is a method of detecting the presence of a SECX nucleic acid molecule in a sample from a mammal, e.g., a human. The method includes contacting the sample with a nucleic acid probe or primer which selectively hybridizes to the nucleic acid molecule and determining whether the nucleic acid probe or primer binds to a nucleic acid molecule in the sample. Binding of the nucleic acid probe or primer indicates the nucleic acid molecule is present in the sample.

The invention further includes a method for detecting or diagnosing the presence of a disease associated with altered levels of a SECX nucleic acid in a sample from a mammal, e.g,. a human. The method includes measuring the level of the nucleic acid in a biological sample from the mammalian subject and comparing the level detected to a level of the nucleic acid present in normal subjects, or in the same subject at a different time. An increase or decrease in the level of the nucleic acid as compared to normal levels indicates a disease condition.

The invention also includes a method of treating a pathological state in a mammal, e.g,. a human, by administering to the subject a SECX polypeptide to the subject in an amount sufficient to alleviate the pathological condition. The polypeptide has an amino acid sequence at least 80% identical to a SECX polypeptide.

Alternatively, the mammal may be treated by administering an antibody as herein described in an amount sufficient to alleviate the pathological condition.

Pathological states for which the methods of treatment of the invention are envisioned include, by non-limiting example, cancer, a neoplastic disorder, an immune disorder, an immune deficiency, an autoimmune disease, acquired immune deficiency syndrome, transplant rejection, allergy, an infection by a pathological organism or agent, an inflammatory disorder, arthritis, psoriasis, a hematopoietic disorder, a skin disorder, a differentiative disorder, atherosclerosis, restenosis, a neurological disease or disorder, Alzheimer's disease, epilepsy, schizophrenia, tissue regeneration, trauma, a surgical or traumatic wound, a spinal cord injury, a corneal dystrophy, a reproductive disorder, a musculature disorder, and a skeletal disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWING

FIG. 1 provides the IL17-like nucleic acid sequence (SEQ ID NO:1) of clone 2191999 and the polypeptide (SEQ ID NO:2) encoded thereby.

FIG. 2 provides the nucleic acid sequence (SEQ ID NO:3) of clone 11753149.0.6 and the polypeptide (SEQ ID NO:4) encoded thereby.

FIG. 3 provides the nucleic acid sequence (SEQ ID NO:5) of clone 11753149.0.37 and the polypeptide (SEQ ID NO:6) encoded thereby.

FIG. 4 provides the nucleic acid sequence (SEQ ID NO:7) of clone 3883556 and the polypeptide (SEQ ID NO:8) encoded thereby.

FIG. 5 provides the nucleic acid sequence (SEQ ID NO:9) of clone 4301136-1 and the polypeptide (SEQ ID NO: 10) encoded thereby.

FIG. 6 provides the nucleic acid sequence (SEQ ID NO:11) of clone 4301136-2 and the polypeptide (SEQ ID NO:12) encoded thereby.

FIG. 7 provides the nucleic acid sequence (SEQ ID NO:13) of clone 4324229 and the polypeptide (SEQ ID NO:14) encoded thereby.

FIG. 8 provides the nucleic acid sequence (SEQ ID NO: 15) of clone 4324229-2 and the polypeptide (SEQ ID NO:16) encoded thereby.

FIG. 9 provides the nucleic acid sequence (SEQ ID NO:17) of clone AC012614_{—}1.0.123 and the polypeptide (SEQ ID NO:18) encoded thereby.

FIG. 10 provides the nucleic acid sequence (SEQ ID NO:19) of clone 4339264-2 and the polypeptide (SEQ ID NO:20) encoded thereby.

FIG. 11 provides the nucleic acid sequence (SEQ ID NO:21) of clone 4357764-3 and the polypeptide (SEQ ID NO:22) encoded thereby, the nucleic acid sequence (SEQ ID NO:144) and polynucleotide sequence (SEQ ID NO:145) of clone 191998702, and polynucleotide sequence (SEQ ID NO:145) of clone CG52676-02.

FIG. 12 provides the nucleic acid sequence (SEQ ID NO:23) of clone 4391184 and the polypeptide (SEQ ID NO:24) encoded thereby.

FIG. 12 provides the nucleic acid sequence (SEQ ID NO:25) of clone 4437909.0.4 and the polypeptide (SEQ ID NO:26) encoded thereby.

FIG. 13 provides the nucleic acid sequence (SEQ ID NO:27) of clone 4437909.0.55 and the polypeptide (SEQ ID NO:28) encoded thereby.

FIG. 15 is a representation of a Western blot of h11753149 protein secreted by 293 cells.

FIG. 16 is a representation of a Western blot of h4437909 protein secreted by 293 cells.

FIG. 17 is a representation of a Western blot of h4437909 protein expressed in E. coli cells.

FIG. 18 provides the nucleic acid sequence (SEQ ID NO:29) of clone pCDNA3.1-TOPO-3883556-S54 and the polypeptide (SEQ ID NO:30) encoded thereby.

FIG. 19 provides the nucleic acid sequence (SEQ ID NO:31) of clone TA-4437909-S443.

FIG. 20 is a histographic representation of TaqMan results for tissue expression of 3883556, wherein the results from various cell types listed in Table 2 are indicated in Panels A, B and C.

FIG. 21 is a histographic representation of TaqMan results for tissue expression of 4324229, wherein the results from various cell types listed in Table 2 are indicated in Panels A, B and C.

FIG. 22 is a histographic representation of TaqMan results for tissue expression of 4339264, wherein the results from various cell types listed in Table 2 are indicated in Panels A, B and C.

FIG. 23 is a histographic representation of TaqMan results for tissue expression of 4391184, wherein the results from various cell types listed in Table 2 are indicated in Panels A, B and C.

FIG. 24 is a listing of the relative expression profiles of clone 4324229-2 in various tissues.

FIG. 25 illustrates sequences of various clones of the present invention, i.e., FIG. 25a shows the polynucleotide sequence of CG52643-02 (SEQ ID NO:85), FIG. 25b shows the polynucleotide sequence of 259341359 (SEQ ID NO:86), FIG. 25c illustrates a ClustalW alignment between the polypeptide sequences encoded by CG52643-02 (SEQ ID NO:87) and 259341359 (SEQ ID NO:88), FIG. 25d shows the polynucleotide sequence of 268824728 (SEQ ID NO:123), FIG. 25e shows the polynucleotide sequence of 268825987 (SEQ ID NO:124), FIG. 25f shows the polynucleotide sequence of 268825997 (SEQ ID NO:125), FIG. 25g shows the polypeptide sequence of 268824728 (SEQ ID NO:126), FIG. 25h shows the polypeptide sequence of 268825987 (SEQ ID NO:127), FIG. 25i shows the polypeptide sequence of 268825997 (SEQ ID NO:128), FIG. 25j shows the polynucleotide and polypeptide sequences of 275698334 (SEQ ID NO:147 and SEQ ID NO:148).

FIG. 26 illustrates the polynucleotide (SEQ ID NO:149) and polypeptide sequence (SEQ ID NO:150) of clone CG52703-03.

### DETAILED DESCRIPTION OF THE INVENTION

The invention provides novel nucleotides and polypeptides encoded thereby. The polynucleotides and their encoded polypeptides are disclosed in SEQ ID NOS:1-30, 85-88, 123-127, and 144-148.

The sequences are collectively referred to as “SECX nucleic acids” or SECX polynucleotides” and the corresponding encoded polypeptide is referred to as a “SECX polypeptide” or SECX protein”. For example, a SECX nucleic acid according to the invention is a nucleic acid including a SECX nucleic acid, and a SECX polypeptide according to the invention is a polypeptide that includes the amino acid sequence of a SECX polypeptide. Unless indicated otherwise, “SECX” is meant to refer to any of the novel sequences disclosed herein.

Table 1 provides a summary of the SECX nucleic acids and their encoded polypeptides. Nucleic acid sequences and polypeptide sequences for SECX nucleic acids according to the invention is provided in the following section of the specification entitled “Disclosed Sequences of SECX Nucleic Acid and Polypeptide Sequences.”

TABLE 1
SECX Clones, Listings, and SEQ ID NOS.

| | | Nucleic Acid | Polypeptide |
|---|---|---|---|
| Clone | Listing | SEQ ID NO: | SEQ ID NO: |
| 2191999 | | 1 | 2 |
| 11753149.0.6 | | 3 | 4 |
| 11753149.0.37 | | 5 | 6 |
| 3883556 | | 7 | 8 |
| 4301136-1 | | 9 | 10 |
| 4301136-2 | | 11 | 12 |
| 4324229 | | 13 | 14 |
| 4324229-2 | | 15 | 16 |
| AC012614_1.0.123 | | 17 | 18 |
| 4339264-2 | | 19 | 20 |
| 4357764-3b | | 21 | 22 |
| 4391184 | | 23 | 24 |
| 4437909.0.4 | | 25 | 26 |
| 4437909.0.55 | | 27 | 28 |
| pCDNA3.1-TOPO- | | 29 | 30 |
| 3883556-S54 | | | |
| CG52643-02 | | 85 | 87 |
| 259341359 | | 86 | 88 |
| 268824728 | | 123 | 126 |
| 268825987 | | 124 | 127 |
| 268825997 | | 125 | 128 |
| 275698334 | | 147 | 148 |
| 191998702 | | 144 | 145 |
| CG52676-02 | | n/a | 146 |
| CG52703-03 | | 149 | 150 |

A polypeptide or protein described herein is the product of a naturally occurring polypeptide or precursor form or proprotein. The naturally occurring polypeptide, precursor or proprotein includes, e.g., the full length gene product, encoded by the corresponding gene. Alternatively, it may be defined as the polypeptide, precursor or proprotein encoded by an open reading frame described herein. A “mature” form of a polypeptide or protein arises as a result of one or more naturally occurring processing steps as they may occur within the cell, or host cell, in which the gene product arises, e.g., cleavage of the amino-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining. Alternatively, a mature form arising from a precursor polypeptide or protein having residues 1 to N, in which an amino-terminal signal sequence from residue 1 to residue M is cleaved, would have the residues from residue M+l to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or the combination of any of them.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

### 1. Clone 2191999

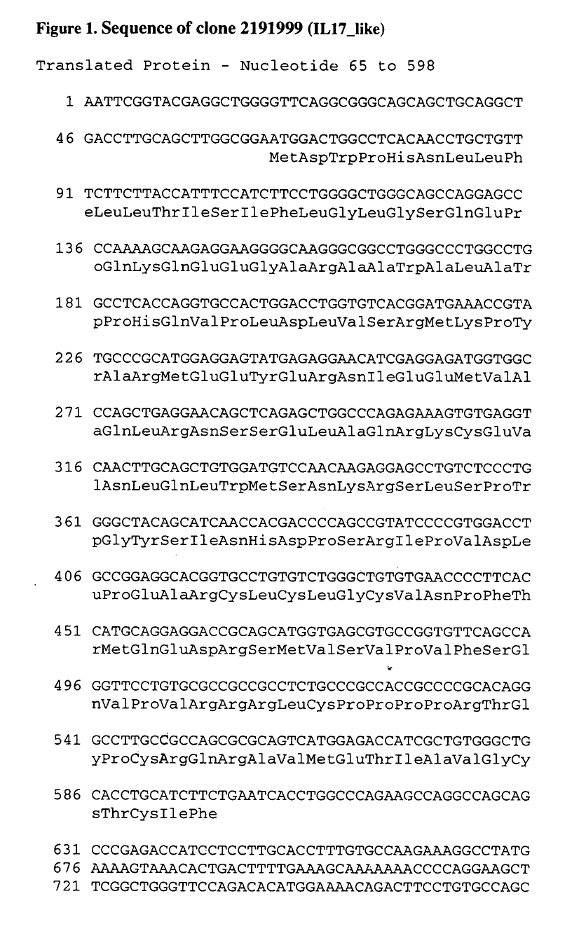

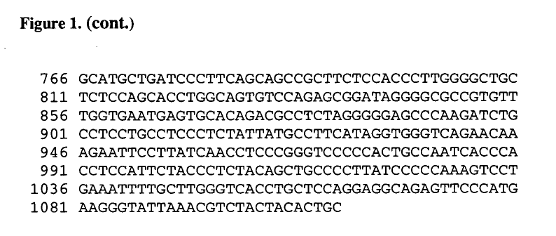

Clone 2191999 resembles interleukin-17 (IL-17). The Clone 2191999 nucleotide sequence (SEQ ID NO:1), shown in FIG. 1, is 1107 bp in length. This nucleotide sequence has an open reading frame (“ORF”) encoding a polypeptide of 178 amino acid residues (represented in FIG. 1; SEQ ID NO:2). The start codon is at nucleotides 65-67 and the stop codon is at nucleotides 599-601. The protein of SEQ ID NO:2 is predicted by the PSORT program to localize extracellularly with a certainty of 0.4037. The program SignalP predicts an N-terminal signal peptide, with the most likely cleavage site between residues 22 and 23, represented by the dash between the amino acids GSQ-EP (i.e., GlySerGln-GluPro).

IL-17 is a T cell-derived cytokine that may play an important role in the initiation or maintenance of the proinflammatory response. Whereas expression of L-17 is restricted to activated T cells, the IL-17 receptor is found to be widely expressed, a finding consistent with the pleiotropic activities of IL-17. Two human cytokines, IL-17B and IL-17C are related to IL-17 (approximately 27% amino acid identity; Proc Natl Acad Sci USA 2000, 97(2):773-8). IL-17B mRNA is expressed in adult pancreas, small intestine, and stomach. IL-17C mRNA is not detected by RNA blot hybridization of several adult tissues. No expression of IL-17B or IL-17C mRNA is found in activated T cells. In a survey of cytokine induction, IL-17B and IL-17C stimulate the release of tumor necrosis factor alpha and IL-1 beta from the monocytic cell line THP-1, whereas IL-17 has a small effect in this system. No induction of IL-1 alpha, IL-6, IFN-gamma, or granulocyte colony-stimulating factor is found in THP-1 cells. Fluorescence-activated cell sorter analysis shows that IL-17B and IL-17C bind to THP-1 cells. Conversely, IL-17B and IL-17C are not active in an IL-17 assay or the stimulation of IL-6 release from human fibroblasts and do not bind to the human IL-17 receptor extracellular domain. These data show that there is a family of IL-17-related cytokines differing in patterns of expression and proinflammatory responses that may be transduced through a cognate set of cell surface receptors. Because Clone 2191999 is highly related to IL-17, by analogy Clone 2191999 may be utilized in assessing patterns of cytokine expression and in proinflammatory responses

In a BLASTX search the nucleotide sequence of SEQ ID NO:1, Clone 2191999 protein is found to be similar to human PRO1031 protein (90%; WO9946281-A2, published 16 Sep. 1999), an expressed sequence tag. It is also 90% similar to human interleukin-17D having 180 residues (WO9935267-A1, published 15 Jul. 1999).

Human IL-17D-like polypeptides are significantly related to human IL-17 polypeptides. The homology between IL-17 and IL-17D suggests that an IL-17D-like polypeptide, e.g., Clone 2191999, is capable of signaling through cytokine receptors. IL-17D-like protein can also be used as a therapeutic agent for the treatment of diseases mediated by IL-17D. IL-17D-like polypeptides bind to B cells. It is likely that these polypeptides can be used for targeting compounds to B cells and B cell tumors, and for specific selection of B cell populations.

BLASTX of SEQ ID NO:1 further shows that Clone 2191999 protein is 90% similar to human embryo derived interleukin related factor I protein (EDIRF I) having 180 residues (WO9932632-A1, published 1 Jul. 1999). The EDIRF-like DNA and protein sequences (e.g., Clone 2191999) and their homologues, antibodies (Ab) specific for EDIRF-like protein, and other modulators may be used: (i) in screening and detection assays, e.g. for chromosome mapping, tissue typing or forensic studies; (ii) in diagnosis, prognosis or monitoring clinical trials; and (iii) for treating or preventing EDIRF-like-related diseases (especially immune, hematopoietic, differentiative, developmental or inflammatory disease, including arthritis and psoriasis. The EDIRF-like coding sequence, or its fragments, are also useful as probes and primers (for detecting related sequences and disease-associated mutations, also for mutagenesis), for expressing recombinant EDIRF and as source of antisense, ribozyme and peptide nucleic acids for inhibiting translation of EDIRF-derived mRNA. EDIRF-like protein is used to raise Ab (useful for detecting EDIRF, including forms with aberrant post-translational modification, for affinity purification and therapeutically) and to screen for specific modulators (e.g. peptides or peptidomimetics).

Clone 2191999 is also 90% similar to human interleukin-20 (IL-20; WO9903982-A1, published 28 Jan. 1999). The Clone 2191999 sequences represent a human interleukin-20-like gene and gene product that may be used, for example, to treat B-cell neoplasms, including chronic lymphocyte leukemia (CLL) and B-lymphocyte leukemia (BLL), and in anticancer and antiviral treatments. Clone 2191999 may be used to treat immunodeficiencies, e.g. in T- and B-lymphocytes, leukopenia, reduced numbers of leukocytes, immune disorders, e.g. rheumatoid arthritis. Clone 2191999 may also be used to augment the humoral or cellular immune response in vivo to other therapeutic agents coadministered with Clone 2191999, e.g. to enhance the efficacy of viral antigen vaccines, such as HIV. Clone 2191999 may also be useful in immunotherapeutic and anti-inflammation compositions, for the treatment of patients suffering from chemotherapy from bone marrow transplants, to treat corneal damage, keratitis, ulcers, thrombocytopenia, to restore neutrophil and platelet counts in treatment of cancer, to enhance erythropoietic production for treating anemias associated with inflammation, renal failure, AIDS and cancer. Clone 2191999 may be employed to treat hematopoiesis, and to treat sepsis. Agonists and antagonists of Clone 2191999 can also be used.

The protein encoded by SEQ ID NO:1 is also 90% similar to human Zcyto7, a polypeptide of 180 residues (WO9849310-A1, published 5 Nov. 1998), a mammalian cytokine-like factor 7 polypeptide. Therefore, Clone 2191999 may be useful e.g. to promote bone and cartilage growth, e.g. to treat osteoporosis, or in treatment of inflammation, neurodegenerative diseases, and so forth.

Clone 2191999 includes the full length protein disclosed as being encoded by the ORF described herein, as well as any mature protein arising therefrom as a result of the removal of a signal peptide. Clone 2191999 also includes all fragments, analogs, homologs and derivatives of Clone 2191999. Thus the proteins of the invention encompass both the precursors and active forms of Clone 2191999 protein.

### 2. Clone 11753149.0.6

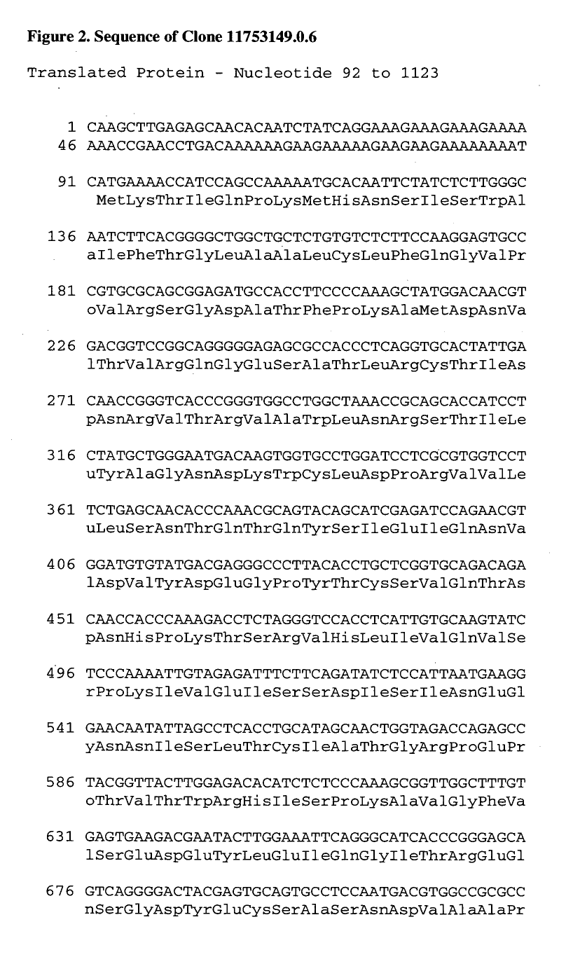

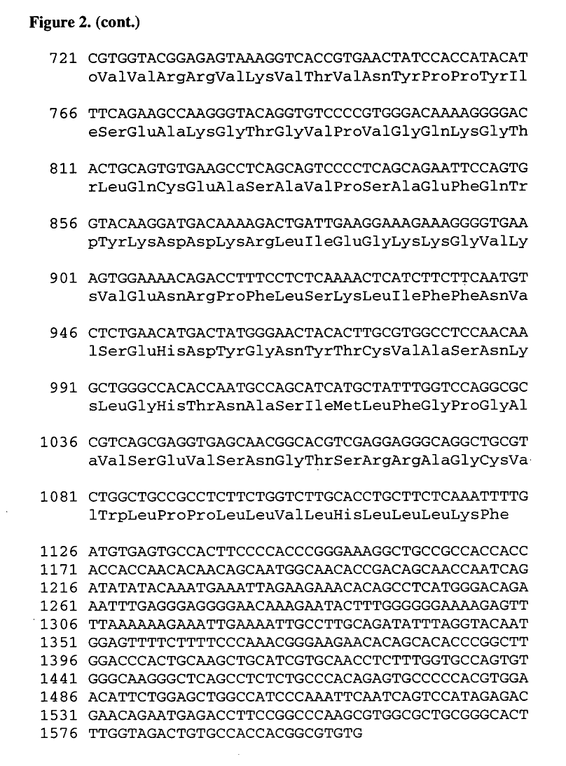

Clone 11753149.0.6 includes a polynucleotide of 1603 bp in length (FIG. 2; SEQ ID NO:3). The differentially expressed gene fragment used in the identification of this clone was obtained from fetal brain tissue. Expressed fragments are also observed in fetal brain and thalamus. Clone 11753149.0.6 includes an ORF encoding a polypeptide of 344 amino acid residues (FIG. 2; SEQ ID NO:4). The ORF contains a predicted N-terminal signal peptide sequence and a C-terminal membrane attachment sequence between residues 327-344. The initiation codon occurs at nucleotides 92-94 and the termination codon at nucleotides 1124-1126. The PSORT predicts that the polypeptide localizes in the plasma membrane with a certainty of 0.8110. The SignalP program predicts that the encoded polypeptide has a signal peptide, and that the most likely cleavage site occurs between residues 33 and 34, represented by the dash between the amino acids VRS-GD (i.e., ValArgSer-GlyAsp). SIGNALP also predicts additional signal peptidase cleavage sites in the segment between residues 18 and 34.

In searches of nucleic acid sequence databases, clone 11753149.0.6 resembles rat neurotrimin, a subfamily of differentially expressed neural cell adhesion molecules, to the extent of 84% identity of 1477 bp in a total sequence of 2040 bp (GenBank-ID:RNU16845|acc:U16845; Struyk et al. J. Neurosci. 15 (3), 2141-2156 (1995)).

Similarities to the additional nucleic acids, described as having similar or analogous properties, were also found, including (1) chicken mRNA for CEPU-1, an immunoglobulin superfamily molecule expressed by developing cerebellar Purkinje cells (GenBank-ID:GGCEPU1|acc:Z72497, Spaltmann and Brummendorf. Neurosci. 16 (5), 1770-1779 (1996));(2) chicken CEPU gene identified as a neural secreted glycoprotein belonging to the immunoglobulin-like opioid binding cell adhesion molecule (OBCAM) subfamily,(GenBank-ID :GGCEPUS|acc:AJ225897, Kim et al., 1999 Mol. Cells 9 (3), 270-276); and (3) Bovine mRNA for opioid binding protein/cell adhesion molecule OBCAM (GenBank-ID:BTOBCAM|acc:X12672).

BLASTP search revealed that the polypeptide encoded by clone 11753149.0.6 has 311 of 336 residues (92%) identical to, and 320 of 336 residues (95%) positive with rat neurotrimin precursor (GP65) having 344 residues (GenBank acc:Q62718). It also has 240 of 337 residues (71%) identical to, and 277 of 337 residues (82%) positive with human opioid binding protein/cell adhesion molecule precursor (OBCAM) having 345 residues (GenBank acc:Q14982).

Neurotrimin-like and/or OBCAM-like proteins of the invention encoded by clone 11753149.0.6 include the full length protein disclosed as being encoded by the ORF described herein, as well as any mature protein arising therefrom as a result of the removal of a signal peptide. Clone 11753149.0.6 also includes all fragments, analogs, homologs and derivatives of Clone 11753149.0.6. Thus the proteins of the invention encompass both the precursors and the active forms of the neurotrimin-like and/or OBCAM-like proteins.

### 3. Clone 11753149.0.37

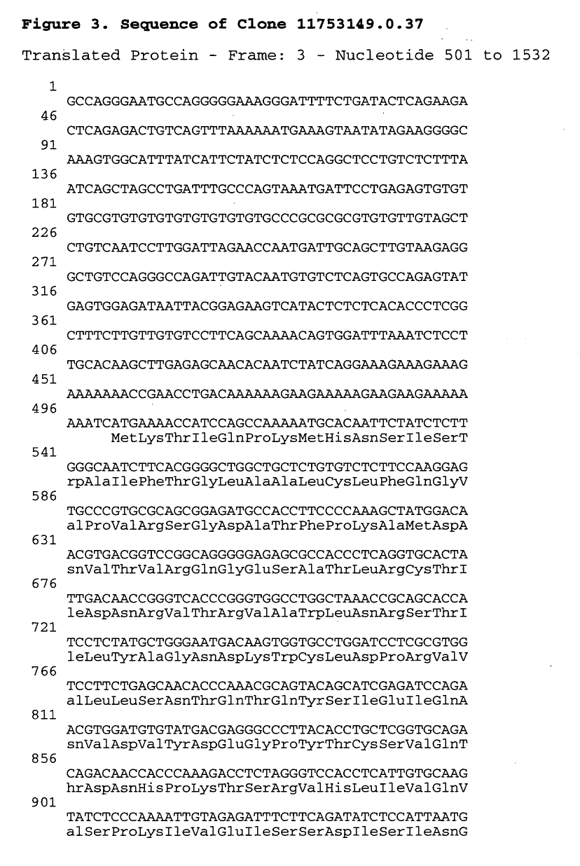

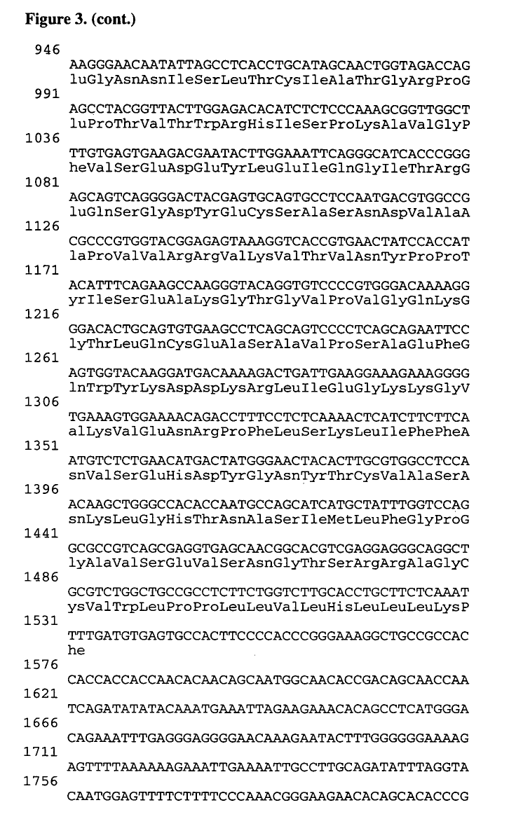

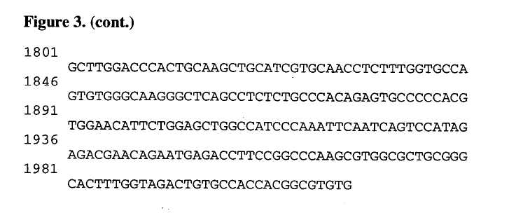

Clone 11753149.0.37 is a variant of clone 11753149.0.6, wherein the nucleotide sequence has a longer 5′ untranslated region (UTR), but the same open reading frame. Clone 11753149.0.37 nucleotide sequence (SEQ ID NO:5) and the predicted polypeptide sequence encoded therein (SEQ ID NOS:6) are given in FIG. 3. The ORF of clone 11753149.0.37 extends from nucleotide 501 to nucleotide 1532, in the numbering scheme of SEQ ID NO:5. The properties of the neurotrimin-like or OBCAM-like polypeptide encoded by clone 11753149.0.37 are the same as those set forth in the preceding section for clone 11753149.0.6. In addition, the long 5′ UTR may include control elements and/or response elements that affect the specificity of expression of the gene product of clone 11753149.0.37 among various tissues, physiological states and pathological conditions.

Neurotrimin-like and/or OBCAM-like proteins of the invention encoded by clone 11753149.0.37 include the full length protein disclosed as being encoded by the ORF described herein, as well as any mature protein arising therefrom as a result of the removal of a signal peptide. Clone 11753149.0.37 also includes all fragments, analogs, homologs and derivatives of Clone 11753149.0.37. Thus the proteins of the invention encompass both the precursors and the active forms of the neurotrimin-like and/or OBCAM-like proteins.

### 4. Clone 3883556

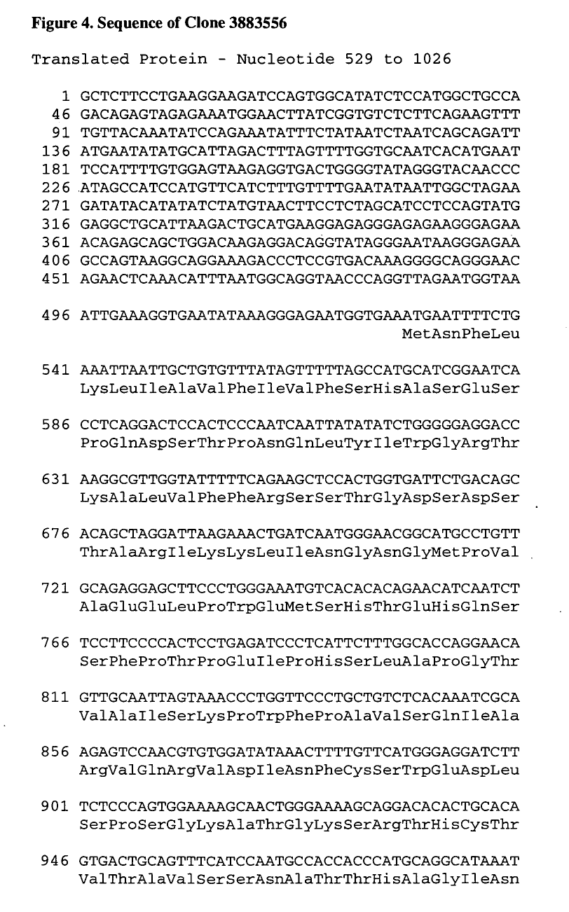

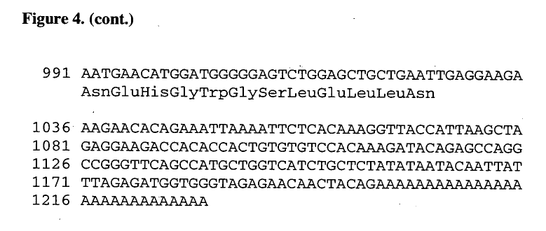

Clone 3883556 includes a polynucleotide of 1228 bp in length (SEQ ID NO:7), shown in FIG. 4. Expression of this sequence is detected in human fetal brain. The polynucleotide of SEQ ID NO:7 encodes a polypeptide of 166 residues (SEQ ID NO:8), shown in FIG. 4, in an ORF beginning with the initiation codon at nucleotides 529-531 and ending at the stop codon at nucleotides 1027-1029. The PSORT program predicts that the polypeptide is localized extracellularly, with a certainty of 0.37. The SignalP program predicts that the polypeptide has a signal peptide with the most likely cleavage site between residues 16 and 17, represented by the dash between the amino acids SHA-SE (i.e., SerHisAla-SerGlu).

BLASTP search revealed a 27% identity, and 41% similarity with ZK899.1—Caenorhabditis elegans, having 161 aa (ACC:Q23659; Z37140); and a 33% identity, and 47% similarity with major merozoite surface antigen—Plasmodium berghei yoelii, 641 aa (fragment) (ACC:G160082).

The protein of the invention encoded by clone 3883556 includes the full length protein disclosed herein, as well as any mature protein arising therefrom as a result of the removal of a signal peptide. Clone 3883556 also includes all fragments, analogs, homologs and derivatives of Clone 3883556. Thus the proteins of the invention encompass both the precursors and the active forms of the 3883556 protein.

### 5. Clone 4301136-1

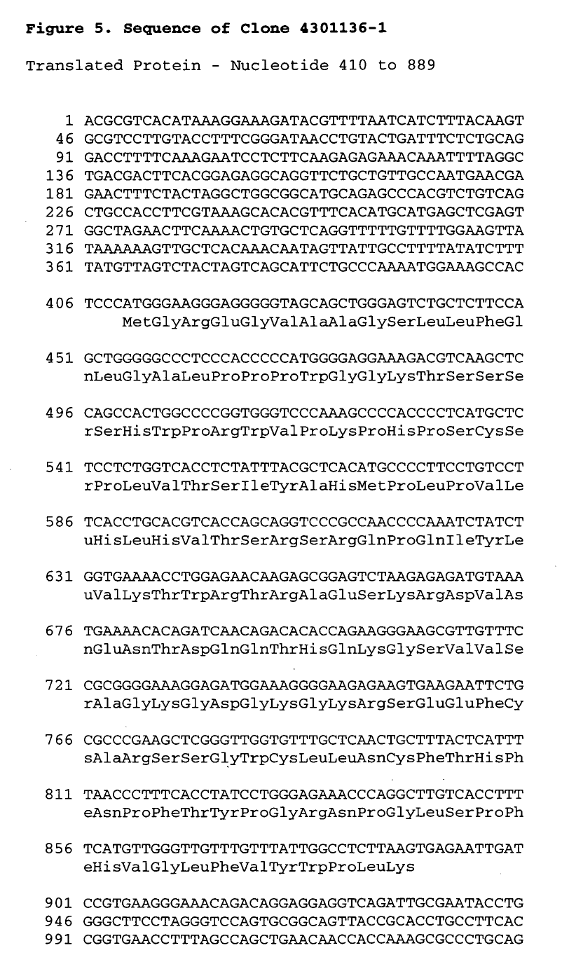

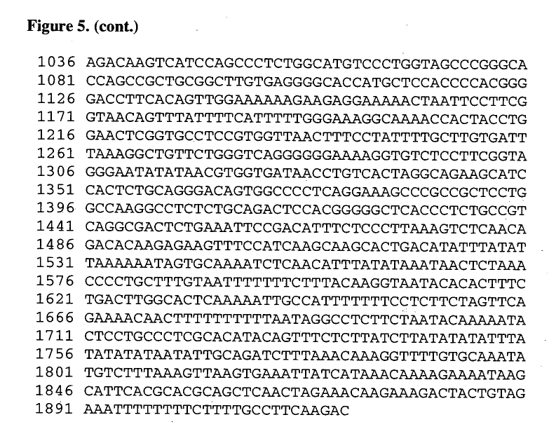

Clone 4301136-1 includes a polynucleotide of 1917 bp (SEQ ID NO:9), as shown in FIG. 5. This clone was initially identified in fetal kidney and heart tissue. The polynucleotide of SEQ ID NO:9 encodes a polypeptide having 160 residues (SEQ ID NO:10), shown in FIG. 5, in an ORF beginning at nucleotides 48-50 and ending with a stop codon at nucleotides 528-530. The PSORT program predicts that the 4301136-1 polypeptide localizes extracellularly with a certainty of 0.3700. The SignalP program predicts only a low probability that the polypeptide contains a known signal peptide. If so, the most likely cleavage site for the signal peptide occurs between residues 23 and 24, represented by the dash between the amino acids PWG-GK (i.e., ProTrpGly-GlyLys).

Clone 4301136-1 proteins of the invention include the full length protein encoded by the ORF disclosed herein, as well as any mature protein arising therefrom, for example, as a result of the removal of a signal peptide. Clone 4301136-1 also includes all fragments, analogs, homologs and derivatives of Clone 4301136-1. Thus the proteins of the invention encompass both the precursors and the active forms of the PCK1-like Clone 4301136-2 protein.

### 6. 4301136-2

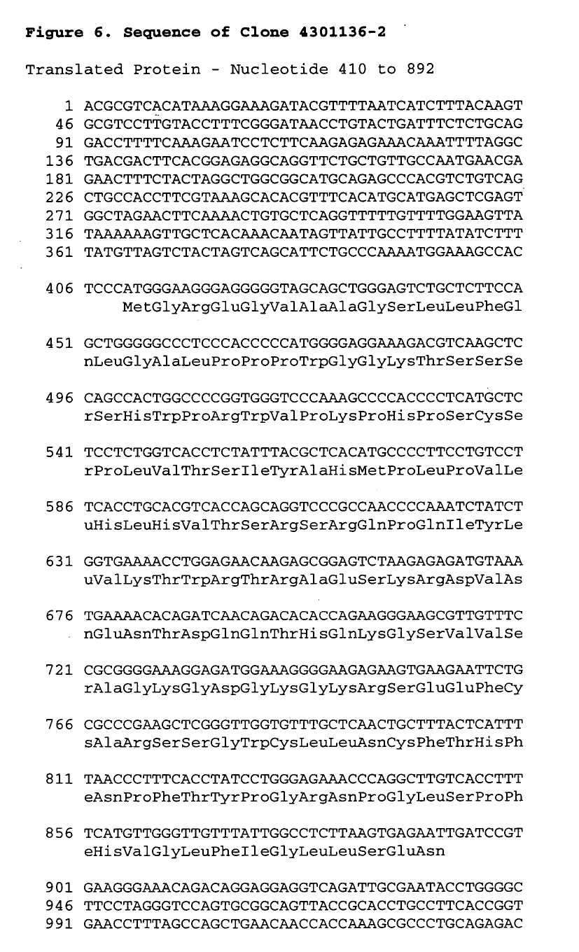

Clone 4301136-2 includes a polynucleotide of 1279 bp (SEQ ID NO:11), shown in FIG. 6. This clone was initially isolated from fetal kidney and heart tissues. It is also found in other tissues, including normal adult lung, osteosarcoma, lymph node tissue, prostate gland, thymus gland, and fetal brain.

The polynucleotide of clone 4301136-2 includes an ORF encoding a polypeptide of 161 residues (SEQ ID NO:12), shown in FIG. 6, with an initiation codon at nucleotides 61-63 and a termination codon at nucleotides 544-546. The PSORT program predicts that this polypeptide localizes extracellularly with a certainty of 0.3700. The SignalP program predicts only a low probability that the polypeptide contains a known signal peptide. If so the most likely cleavage site occurs between residues 23 and 24, represented by the dash between the amino acids PWG-GK (i.e., ProTrpGly-GlyLys).

Clone 4301136-2 includes the full length protein encoded by the ORF disclosed herein, as well as any mature protein arising therefrom, for example, as a result of the removal of a signal peptide. Clone 4301136-2 also includes all fragments, analogs, homologs and derivatives of Clone 4301136-2. Thus the proteins of the invention encompass both the precursors and the active forms of Clone 4301136-2 protein.

### 7. 4324229

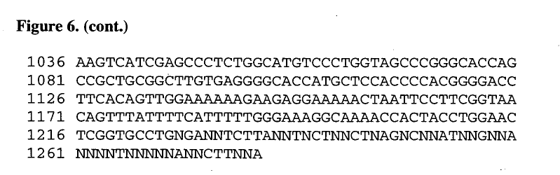

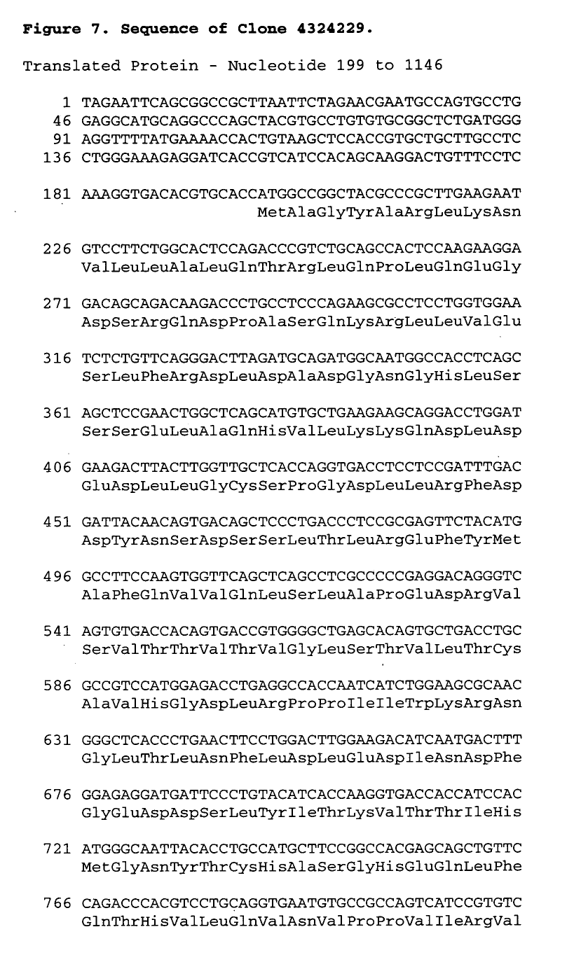

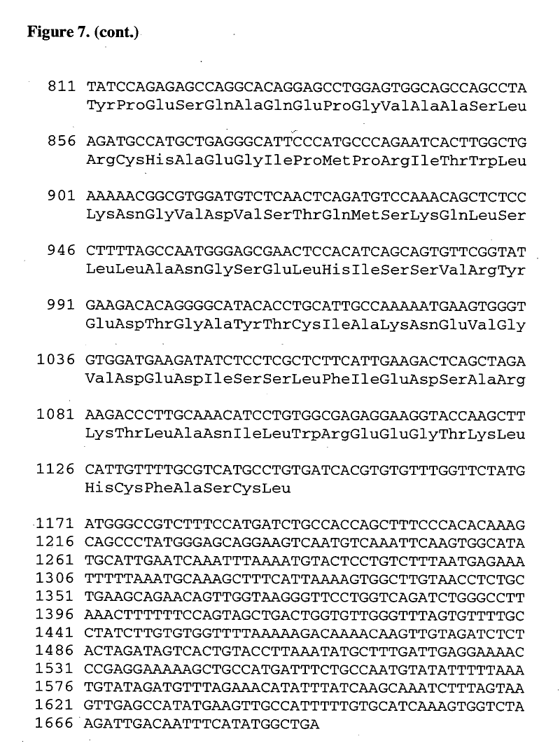

Clone 4324229 includes a polynucleotide of 1689 bp (SEQ ID NO:13), shown in FIG. 7. This clone is shorter in both the 5′ and 3′ directions than the nucleotide sequence of clone 4324229-2 disclosed in the next section. It also is closely related to the nucleotide sequence of clone AC012614-1.0.123 disclosed below. This sequence was originally identified in lymph node. The Clone 4324229 polypeptide has 316 amino acid residues (SEQ ID NO:14), represented in FIG. 7, and is encoded by an ORF beginning at nucleotides 199-201 of SEQ ID NO:13, with a termination codon at nucleotides 1147-1149. The PSORT program predicts that the polypeptide localizes to the mitochondrial matrix space with a certainty of 0.4433. The SignalP program predicts that this sequence most likely has no known signal sequence. SignalP does predict, however, that if a signal sequence is present, the most likely cleavage site occurs between residues 18 and 19, represented by the dash between the amino acids TRL-QP (i.e., TryArgLeu-GlnPro).

Database similarity searches indicate that the protein encoded by clone 4324229 has similarity to a fragment of human limbic system associated membrane protein (LAMP; PCT Publication WO9630052-A1, published 3 Oct. 1996). LAMP is a self-binding, antibody-like cell surface adhesion protein involved in formation of connections between adjacent neurons. LAMP protein, and by analogy the clone 4324229 protein, may be important in nerve growth and differentiation, epilepsy, Alzheimer's disease and schizophrenia.

The protein encoded by clone 4324229 is also similar to portions of human Down syndrome-cell adhesion molecule (DS-CAM2), a protein of 1571 residues (PCT Publication WO9817795-A1, published 30 Apr. 1998). DS-CAM2 is a soluble extracellular protein belonging to a novel subclass of the Ig superfamily, with highest homology to neural cell adhesion molecules. DS-CAM polypeptides are associated with developmental and neurological processes. DS-CAM polypeptides, and by analogy the clone 4324229 polypeptides, can be used in, e.g. neural prosthetic devices used in entubulation methods of repairing (regenerating) damaged or severed peripheral nerves, and in bioassays to identify agonists and antagonists to said proteins and processes. The clone 4324229 polypeptides can also be used in detection, diagnosis and therapy of developmental and neurological abnormalities such as Down syndrome, mental retardation, holoprosencephaly, agenesis of the corpus callosum, or schizencephaly.

In a BLASTN similarity search, a 895 bp portion of the clone 4324229 nucleotide sequence was found to be 100% identical to the sequence of human mRNA for KIAA1061 protein (GenBank-ID:AB028984|acc:AB028984, submitted 17 Jun. 1999). KIAA1061 originates in brain and its sequence falls within the ORF identified above for clone 4324229. A BLASTP similarity was found to FRAZZLED of Drosophila melanogaster (ACC:Q94537).

Clone 4324229 proteins include the full length protein encoded by the ORF disclosed herein, as well as any mature protein arising therefrom. Such a mature protein could be formed, for example, as a result of the removal of a signal peptide. Clone 4324229 also includes all fragments, analogs, homologs and derivatives of Clone 4324229. Thus the proteins of the invention encompass both the precursors and the active forms of Clone 4324229 protein.

### 8. 4324229-2

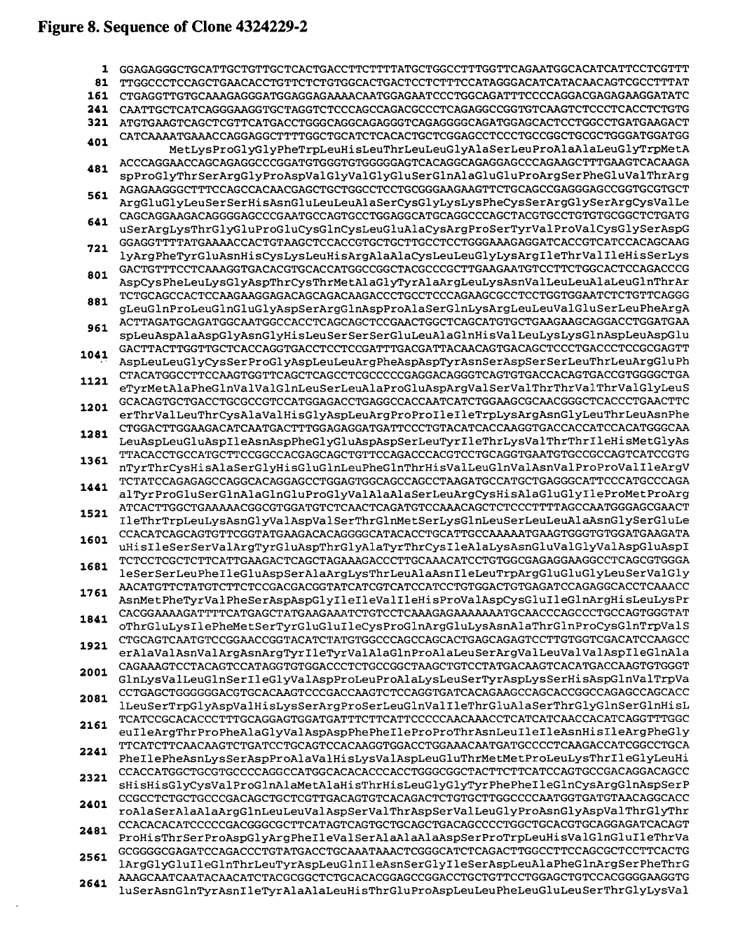

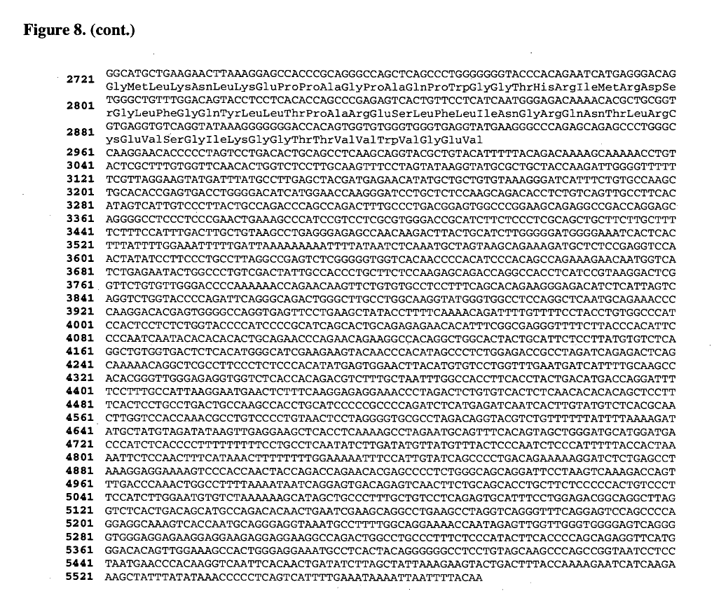

Clone 4324229-2 includes a polynucleotide of 4000 bp (SEQ ID NO:15), shown in FIG. 8. This clone incorporates extensions in both the 5′ and 3′ directions of the nucleotide sequence of clone 4324229 disclosed above. It also is closely related to the nucleotide sequence of clone AC012614-1.0.123 disclosed in the following section. Clone 4324229-2 was originally identified in lymph node. Clone 4324229-2 nucleotide sequence encodes a polypeptide of 842 amino acid residues (SEQ ID NO:16), shown in FIG. 8. This polypeptide is encoded by an open reading frame beginning at nucleotides 408410, with a termination codon at nucleotides 2934-2936.

BLASTX analysis indicates that a portion of the C-terminus of Clone 4324229-2 protein is identical to KIAA1061. See DNA Res. 6:197-205(1999); GenBank-ID:AB028984|acc:AB028984. It is similar to cell adhesion molecules and follistatin-like proteins.

Clone 4324229-2 proteins include the full length protein encoded by the ORF disclosed herein, as well as any mature protein arising therefrom. Such a mature protein could be formed, for example, as a result of the removal of a signal peptide. Clone 4324229-2 also includes all fragments, analogs, homologs and derivatives of Clone 4324229-2. Thus the proteins of the invention encompass both the precursors and the active forms of a LAMP-like and DS-CAM-like Clone 4324229-2 protein.

### 9. AC012614_{—}1.0.123

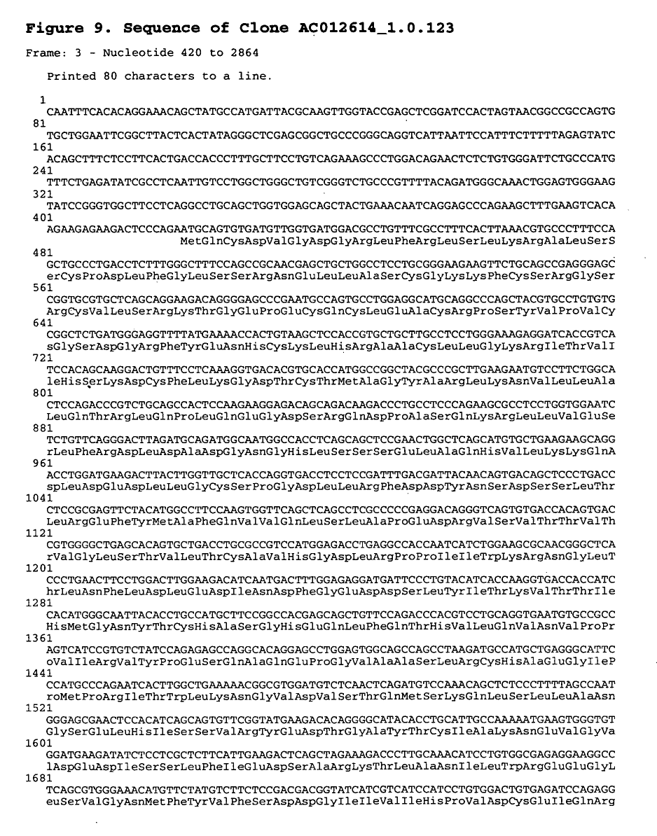

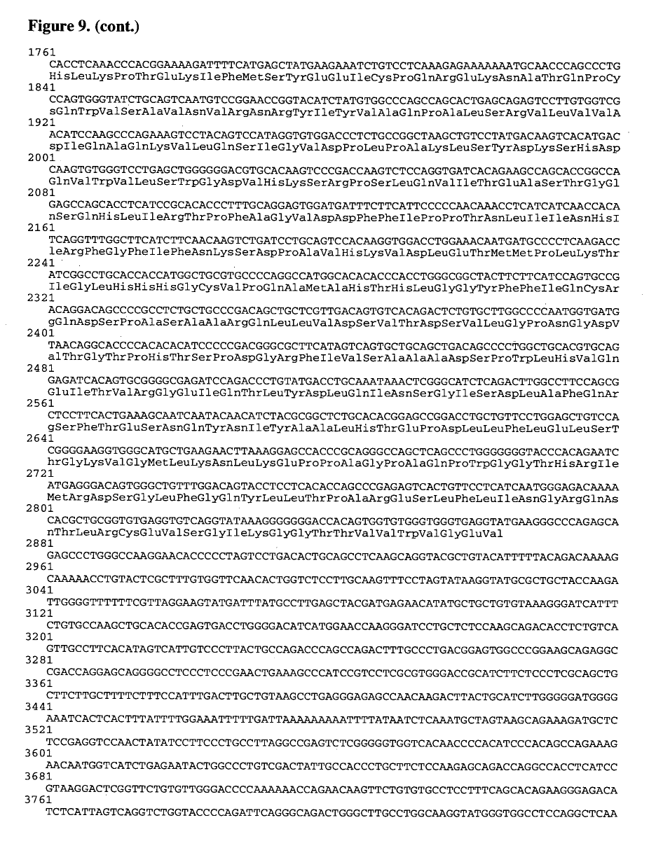

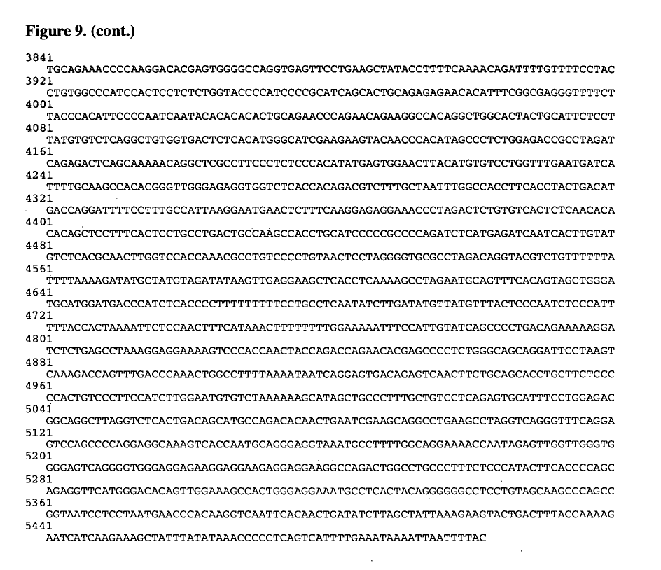

AC012614_{—}1.0.123 includes a full-length clone of 5502 nucleotides (SEQ ID NOS: 17) and the entire coding sequence of a predicted 815 amino acid protein (SEQ ID NOS: 18), shown in FIG. 9. The predicted ORF spans from nucleotides 420 to 2865. This sequence is expressed in glioma, osteoblast, other cancer cells, lung carcinoma, and small intestine.

AC012614_{—}1.0.123 maps to the Unigene entry 123420, which is expressed in brain, breast, kidney, pancreas, and pooled tissue. This entry further maps to the Chromosome 5 marker stSG63086 (also known as RH104076) (GM99-GB4 Map information); Position: 510.63 (cR3000); Lod score: 0.71; Reference Interval: D5S471-D5S393 (129.6-140.8 cM). By integrating the marker information with the MIM gene map, it is believed clone AC012614_{—}1.0.123 maps to 5q21-5q31.

AC012614_{—}1.0.123 was searched against other databases using SignalPep and PSort search protocols. The protein is most likely located in the mitochondrial matrix space (certainty=0.4718) and seems to have no known N-terminal signal sequence. The predicted molecular weight is 90346.9 daltons.

The predicted AC012614_{—}1.0.123 amino acid sequence was searched in the publicly available GenBank database using BLASTP. The 815 residue AC012614_{—}1.0.123 protein shows 100% identity (693 of 693 amino acids) with the 693 aa human KIAA1061 protein fragment (ACC:BAA83013). AC012614_{—}1.0.123 protein is similar to cell adhesion molecules; to murine, rat, Xenopus and human follistatin-related protein precursor (TGF-beta-inducible protein TSC-36, a protein of about 300 residues in the various species); and to short segments of Drosophila roundabout and frazzled. These genes are involved in neuronal development and reproductive physiology. By analogy, AC012614_{—}1.0.123 proteins and polypeptides may also be involved in neuronal development and reproductive physiology.

Frazzled encodes a Drosophila member of the DCC immunoglobulin subfamily and is required for CNS and motor axon guidance. Cell 87:197-204(1996). Characterization of a rat C6 glioma-secreted follistatin-related protein (FRP) and cloning and sequence of the human homologue is described in Eur. J. Biochem. 225:937-946(1994). This protein may modulate the action of some growth factors on cell proliferation and differentiation. FRP binds heparin. The follistatin-related protein is a secreted protein and has one follistatin-like domain. The cloning and early dorsal axial expression of Flik, a chick follistatin-related gene and evidence for involvement in dorsalization/neural induction is described in Dev. Biol. 178:327-342(1996). Roundabout controls axon crossing of the CNS midline and defines a novel subfamily of evolutionarily conserved guidance receptors, as shown in Cell 92:205-215(1998). cDNA cloning and structural analysis of the human limbic-system-associated membrane protein (LAMP) is described in Gene 170:189-195(1996). LAMP, a protein of the OBCAM family that contains three immunoglobulin-like C2-type domains, mediates selective neuronal growth and axon targeting. LAMP contributes to the guidance of developing axons and remodeling of mature circuits in the limbic system. This protein is essential for normal growth of the hippocampal mossy fiber projection. LAMP is attached to the membrane by a GPI-Anchor. It is expressed on limbic neurons and fiber tracts as well as in single layers of the superior colliculus, spinal chord and cerebellum. Characterization of the human full-length PTK7 cDNA encoding a receptor protein tyrosine kinase-like molecule closely related to chick KLG is disclosed in J. Biochem. 119:235-239(1996). Based upon homology, AC012614-1.0.012 proteins and each homologous protein or peptide may share at least some activity.

The region to which AC012614_{—}1.0.123 maps is listed in the National Center for Biotechnology Information website for the Online Mendelian Inheritance in Man (OMIM™) gene map (URL: “www.ncbi.nlm.nih.gov/Omim/”) to be associated with susceptibility to the following diseases (where available, OMIM™ identifying numbers are underlined): allergy and asthma; hemangioma; capillary infantile Schistosoma mansoni infection; susceptibility/resistance to Spinocerebellar ataxia; bronchial asthma; Plasmodium falciparum parasitemia; intensity of Corneal dystrophy, Groenouw type I (OMIM™ 121900); corneal dystrophy, lattice type I (OMIM™ 122200); Reis-Bucklers corneal dystrophy; corneal dystrophy, Avellino type eosinophilia, familial myelodysplastic syndrome; myelogenous leukemia, acute Cutis laxa, recessive, type I; deafness, autosomal dominant non-syndromic sensorineural; 1 contractural arachnodactyly; congenital neonatal alloimmune thrombocytopenia; glycoprotein Ia deficiency male infertility; Charcot-Marie-Tooth neuropathy, demyelinating Gardner syndrome; adenomatous polyposis coli; colorectal cancer; desmoid disease, hereditary (OMIM™ 135290); Turcot syndrome (OMIM™ 276300); and adenomatous polyposis coli, attenuated. By analogy, clone AC012614_{—}1.0.123 is implicated in at least all of the above mentioned diseases and may have therapeutic uses for these diseases.

Clone AC012614_{—}1.0.123 has similarity to cell adhesion molecules, follistatin, roundabout and frazzled. These genes are involved in neuronal development and reproductive physiology. Therefore Clone AC012614_{—}1.0.123 is also implicated in disorders such as or therapeutic uses for nerve trauma, neurodegenerative disorders, epilepsy, mental health conditions, tissue regeneration in vivo and in vitro, and female reproductive system disorders and pregnancy.

Clone AC012614_{—}1.0.123 proteins include the full length protein encoded by the ORF disclosed herein, as well as any mature protein arising therefrom. Such a mature protein could be formed, for example, as a result of the removal of a signal peptide. Clone AC012614_{—}1.0.123 also includes all fragments, analogs, homologs and derivatives of Clone AC012614_{—}1.0.123. Thus the invention encompass both the precursors and the active forms of a protein encoded by clone AC012614_{—}1.0.123.

### 10. 4339264-2

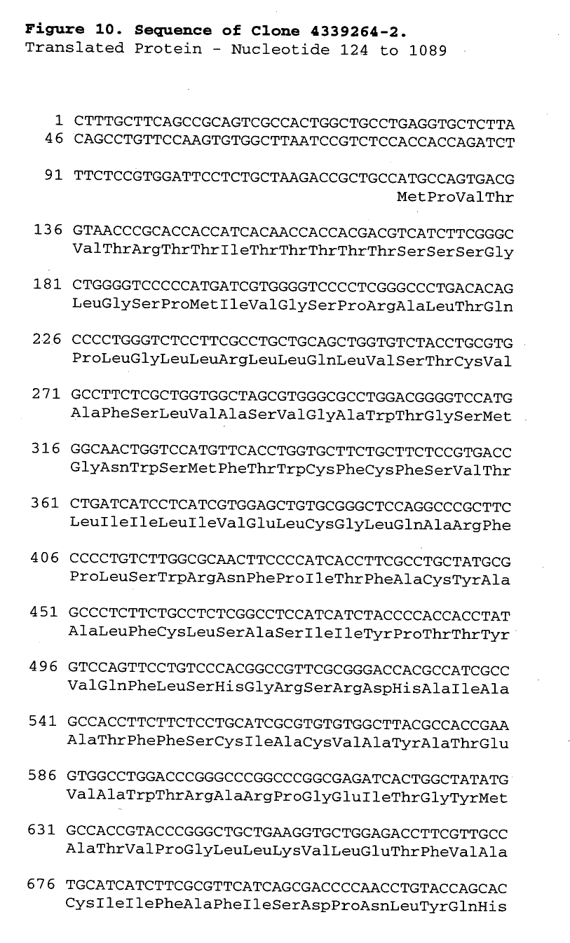

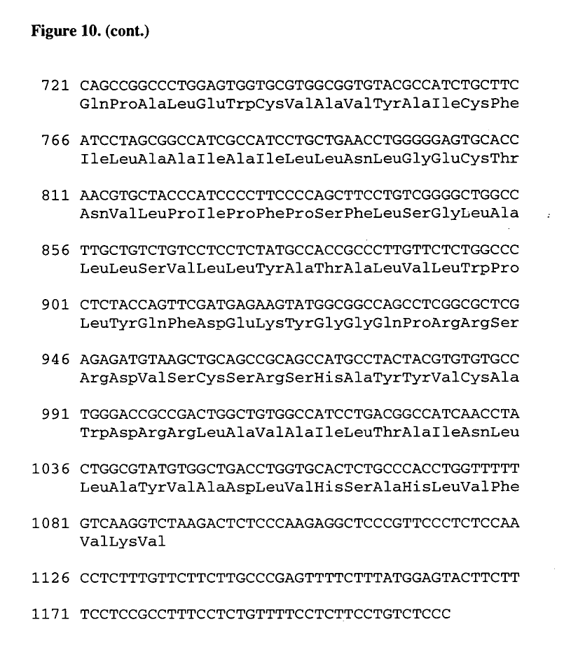

Clone 4339264-2 includes a polynucleotide having 1208 bp (SEQ ID NO:19), shown in FIG. 10. This clone was isolated from lymph node, and is also found in MCF-7, OVCAR-3, heart, prostate, uterus, mammary gland, salivary gland, thalamus, bone marrow, lymph node, spleen, fetal liver, fetal thymus—CRL7046, brain, fetal brain, liver, fetal liver, skeletal muscle, pancreas, kidney, heart, lung and placenta. Clone 4339264-2 includes an ORF encoding a polypeptide of 322 amino acid residues (SEQ ID NO:20), shown in FIG. 7. The initiation codon is found at nucleotides 124-126 and the stop codon is at nucleotides 1090-1092. The PSORT program predicts that the protein localizes to the mitochondrial inner membrane with a certainty of 0.7515, or to the plasma membrane with a certainty of 0.6000. The SignalP program predicts that there may be a signal peptide, with the most likely cleavage site found between residues 59 and 60, represented by the dash between the amino acids VGA-WT (i.e., ValGlyAla-TrpThr).

BLASTX and BLASTP searches show that the protein has an 84 residue fragment that is 100% identical to a the same fragment in a protein encoded by a human EST sequence (PCT Publication WO 9906552-A2, published 11 Feb. 1999). An additional BLASTN search showed that clone 4339264-2 is similar to murine mRNA for myeloid associated differentiation protein (GenBank-ID:MMMYELUPR|acc:AJ001616, submitted 15 Sep. 1997). This gene is described as being expressed in a stage specific fashion during myeloid differentiation but absent in lymphoid cells.

BLASTP searches identified additional similarities to a 296 aa mouse myeloid upregulated protein (GenBank ACC:O35682), and a 153 aa human T-lymphocyte maturation-associated protein (GenBank ACC:P21145).

Clone 4339264-2 proteins include the full length protein encoded by the ORF disclosed herein, as well as any mature protein arising therefrom. Such a mature protein could be formed, for example, as a result of the removal of a signal peptide. Clone 4339264-2 also includes all fragments, analogs, homologs and derivatives of Clone 4339264-2. Thus the proteins of the invention encompass both the precursors and the active forms of 4339264-2, including, for example, a myeloid associated differentiation-like Clone 4339264-2 protein.

### 11. 4357764-3

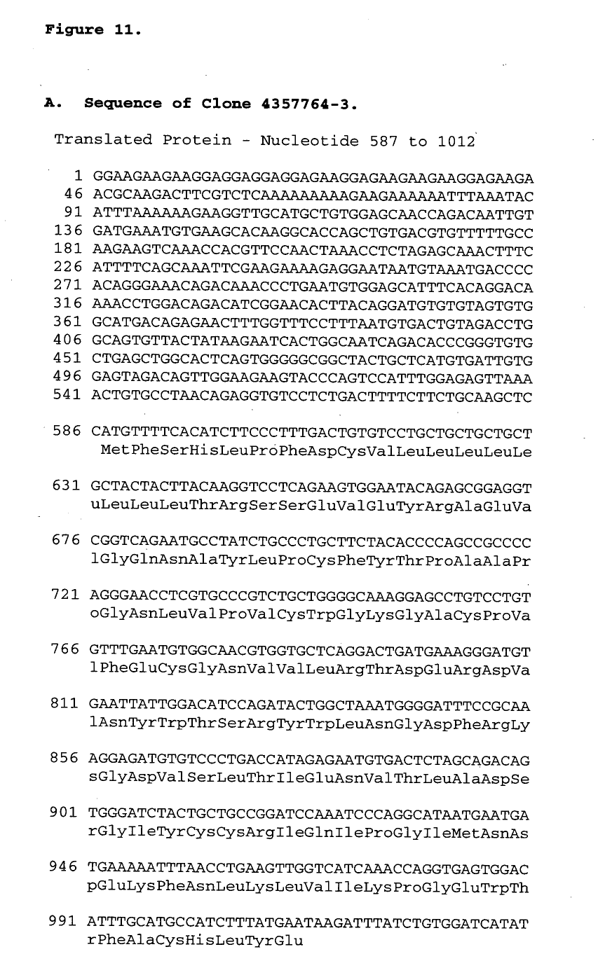

Clone 4357764-3 includes a polynucleotide of 1203 bp (SEQ ID NO:21), shown in FIG. 11. This clone was isolated from lymph node. The clone includes an ORF encoding a polypeptide of 142 amino acid residues (SEQ ID NO:22), shown in FIG. 11. The ORF begins with an initiation codon at nucleotides 587-589 of SEQ ID NO:21 and ends with a stop codon at nucleotides 1013-1015. The PSORT program predicts that the protein is localized extracellularly with a certainty of 0.8200. SignalP predicts that there is a signal peptide present, with the most likely cleavage site found between residues 21 and 22, represented by the dash between the amino acids TRS-SE (i.e., ThrArgSer-SerGlu).

BLASTX analysis showed that, over 135 residues, the polypeptide encoded by clone 4357764-3 is 97% identical and 98% positive with the 301 residue protein encoded by human “200 gene” that is reported to be differentially expressed in T helper cells (U.S. Pat. No. 5,721,351; PCT Publication WO 9627603-A1, published 12 Sep. 1996). The 200 gene protein is reported to be a novel cell surface receptor of the Ig superfamily class. Expression of 200 gene is many-fold higher in TH1 than in TH2 subpopulations (WO9627603-A1). Modulation of the 200 gene product may ameliorate a range of T-cell-related disorders. BLASTP searches also show a moderate degree of similarity to kidney injury molecule-1 of the rat (GenBank acc:O54947) and to human hepatitis A virus cellular receptor 1 (GenBank acc:O43656).

Clone 4357764-3 proteins include the full length protein encoded by the ORF disclosed herein, as well as any mature protein arising therefrom. Such a mature protein could be formed, for example, as a result of the removal of a signal peptide. Clone 4357764-3 also includes all fragments, analogs, homologs and derivatives of Clone 4357764-3. Thus the proteins of the invention encompass both the precursors and the active forms of a protein encoded by clone 4339264-3.

### 12. 4391184

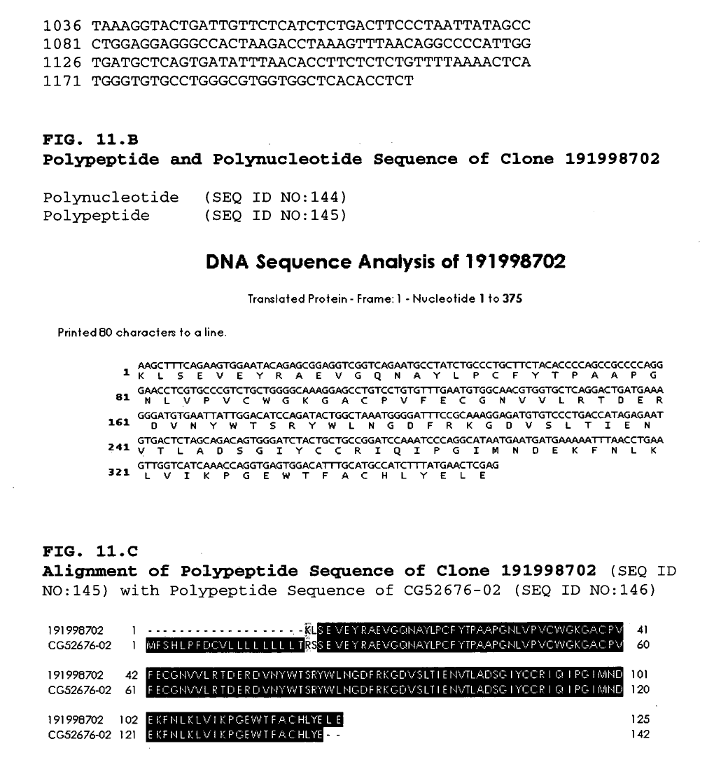

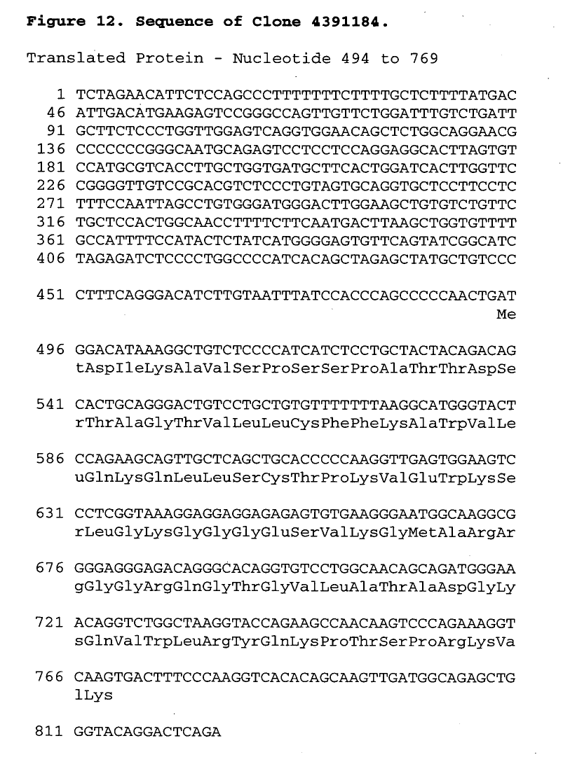

Clone 4391184 is a polynucleotide of 825 bp (SEQ ID NO:23), shown in FIG. 12. This clone was isolated from lymph node tissue. Clone 4391184 contains an ORF encoding a protein of 92 amino acid residues (SEQ ID NO:24), shown in FIG. 12. The start codon is at nucleotides 494496 and the stop codon is at nucleotides 770-772. The PSORT program predicts that the protein of clone 4391184 localizes to the microbody (peroxisome) with a certainty of 0.5690. The SignalP software program predicts a low likelihood of the protein including a known signal peptide.

BLASTP searching revealed a similarity of Clone 4391184 to a fragment of human superoxide dismutase (Cu, Zn) (GenBank ACC:Q16839). Clone 4391184 proteins include the full length protein encoded by the ORF disclosed herein, as well as any mature protein arising therefrom. Such a mature protein could be formed, for example, as a result of the removal of a signal peptide. Clone 4391184 also includes all fragments, analogs, homologs and derivatives of Clone 4391184. Thus the proteins of the invention encompass both the precursors and the active forms of a protein encoded by clone 4391184.

### 13. 4437909.0.4

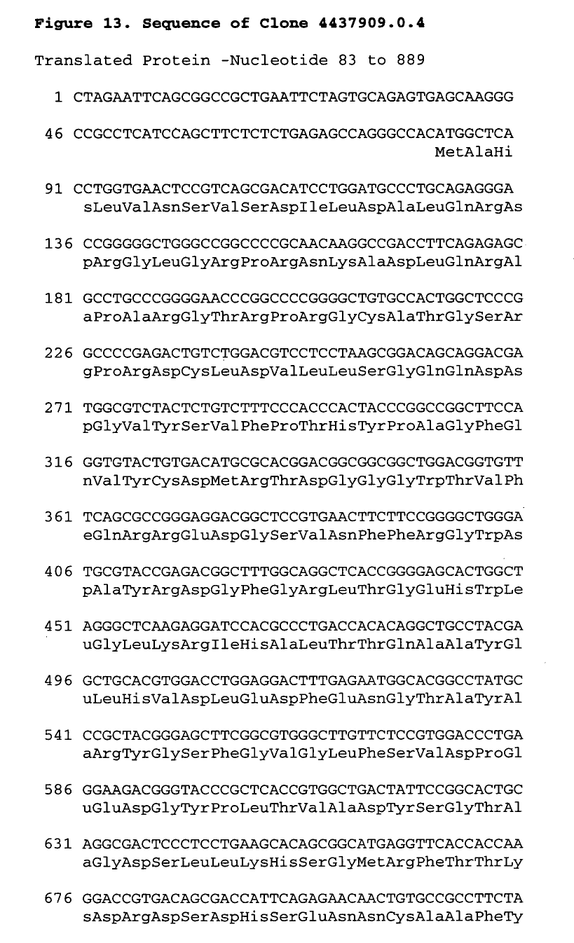

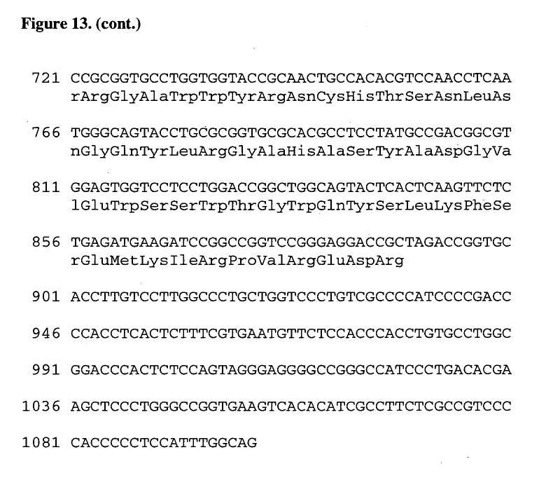

Clone 4437909.0.4 was originally identified by Applicant as clone 4437909 in U.S. Provisional application Ser. No. 60/128,514. Clone 4437909.0.4 includes a polynucleotide of 1099 bp (SEQ ID NO:25), shown in FIG. 13. This clone has been found in osteogenic sarcoma cell lines—HTB96, adrenal gland, thalamus, fetal brain and fetal lung. Clone 4437909.0.4 includes an ORF encoding a polypeptide of 269 amino acid residues (SEQ ID NO:26), shown in FIG. 13. The initiation codon for this polypeptide occurs at nucleotides 83-85 of SEQ ID NO:25 and the termination codon is at nucleotides 890-892. The PSORT program predicts that the 4437909.0.4 protein is localized in the microbody (peroxisome) with a certainty of 0.7480. The SignalP program predicts that there is no known signal peptide.

BLASTP and BLASTX searching showed that the 4437909.0.4 polypeptide has a 55% identity and a 70% similarity to the 255 residue human microfibril-associated glycoprotein 4 (ACC:P55083 and U.S. Pat. No. 5,972,654-A, issued 26 Oct. 1999). The human microfibril-associated glycoprotein 4 splice variant (MAG4V) polypeptides and/or antibodies thereto are disclosed in this patent as being usable to down regulate MAG4V expression and activity. By analogy, Clone 4437909.0.4 polypeptides as well as MAG4V proteins may be used to treat reproductive disorders (e.g. disruptions of the estrous cycle and spermatogenesis, polycystic ovary syndrome and cancers of the prostate and ovaries), muscular disorders (e.g. Duchenne's muscular dystrophy, lipid myopathy and myocarditis), immunological disorders (e.g. Addison's disease, asthma, anemia and acquired immune deficiency syndrome (AIDS)) and neoplastic disorders (e.g. myeloma, sarcoma, leukemia and lung cancer).

BLASTX searching further showed that the 4437909.0.4 protein is 48% identical and 61% positive with the 313 residue human 35 kDa opsonin protein P35 (JP08038182-A, published 13 Feb. 1996). P35 protein has opsonin activity useful in prevention and treatment of infectious diseases. Opsonin activates the phagocytosis of pathogenic microbes by phagocytic cells, useful in the prevention and treatment of infectious diseases. By analogy, Clone 4437909.0.4 may also be utilized in the prevention and treatment of infectious diseases. Additionally the 4437909.0.4 protein, over 221 residues, is also 52% identical and 63% similar to the 324 residue porcine TGF-beta-1 binding protein (WO9222319-A), and Clone 4437909.0.4 protein may additionally have TGF-beta-1 binding activity.

Clone 4437909.0.4 proteins include the full length protein encoded by the 4437909.0.4 ORF disclosed herein, as well as any mature protein arising therefrom. Such a mature protein could be formed, for example, as a result of the removal of a signal peptide. Clone 4437909.0.4 also includes all fragments, analogs, homologs and derivatives of Clone 4437909.0.4. Thus the proteins of the invention encompass both the precursors and the active forms of a protein encoded by clone 4437909.0.4. Clone 4437909.0.4 activities may include those activities possessed by MAG4V protein or opsonin P35 protein.

### 14. 4437909.0.55

Clone 4437909.0.55 is a variant of clone 4437909.0.4, and has a shorter 5′ UTR and two base changes in the coding sequence.

Clone 4437909.0.55 has 1054 bp (SEQ ID NO:27), shown in FIG. 14. This clone has been found in osteogenic sarcoma cell lines—HTB96, adrenal gland, thalamus, fetal brain and fetal lung. This clone includes an ORF beginning at nucleotides 3840 and terminating at nucleotides 845-847, encoding a polypeptide of 269 amino acid residues (SEQ ID NO:28), shown in FIG. 14. The mutations in the open reading frame lead to two mutated amino acid residues. The properties and physiological activities of clone 4437909.0.55 are essentially the same as those summarized above for clone 4437909.0.4

Clone 4437909.0.55 proteins include the full length protein encoded by the 4437909.0.55 ORF disclosed herein, as well as any mature protein arising therefrom. Such a mature protein could be formed, for example, as a result of the removal of a signal peptide. Clone 4437909.0.55 also includes all fragments, analogs, homologs and derivatives of Clone 4437909.0.55. Thus the proteins of the invention encompass both the precursors and the active forms of a protein encoded by clone 4437909.0.55. Clone 4437909.0.55 activities may include those activities possessed by MAG4V protein or opsonin P35 protein.

### 15. CG52703-03

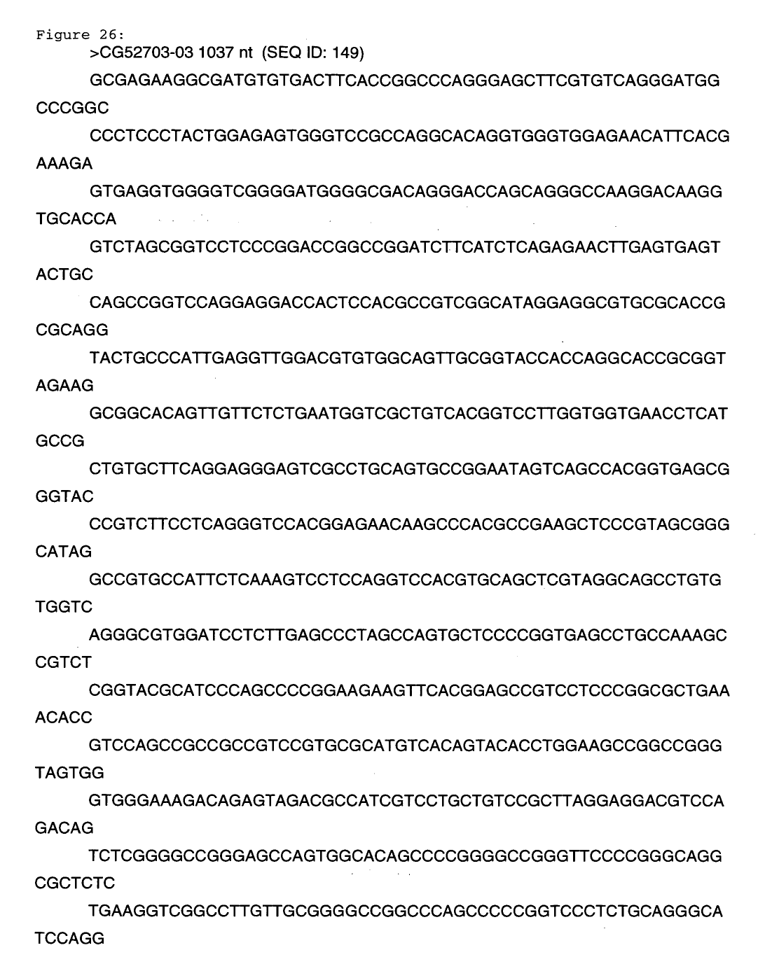

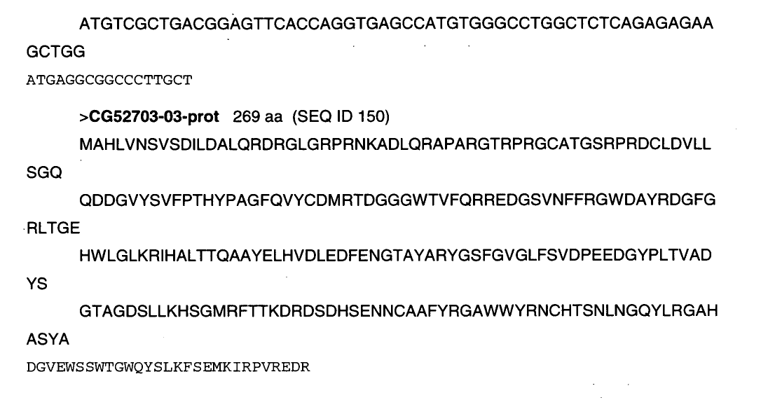

Clone CG52703-03 has 1037 bp, and the polynucleotide (SEQ ID NO: 149) and polypeptide (SEQ ID NO:150) are given at FIG. 26. The polynucleotide sequence encodes a microfibril-associated glycoprotein-like protein. An open reading frame was identified beginning at nucleotides 46-48 and ending at nucleotides 853-855. The encoded polypeptide has 269 amino acid residues, and is presented in FIG. 26 using the one letter codes. CG52703-03 has greater than 65% nucleic acid identity to human microfibril associated glucoprotein 4, and has greater than 55% identity to the same protein. The polypeptide displays at least one fibrinogen beta and gamma chains C-terminal globular domain, and thus has properties similar to those of other proteins known to contain these domains. The nucleic acid maps to chromosome 9. Expression of the gene has been detected in at least the following tissues: adrenal/supraadrenal gland, salivary gland, liver, bone marrow, lymphoid tissue, hippocampus, lung, thalamus, amygdala, placenta and hair follicles. The polypeptide is localized to peroxisomal bodies and is also secreted. The protein has structural and physiological functions similar to those of the microfibril associated glycoprotein family. Clone CG52703-03 activities include those activities possessed by microfibril associated glycoprotein families, and accordingly, is useful in serving as a diagnostic or prognostic marker for the diseases indicated herein. Potential therapeutic applications include protein therapeutics, small molecule targets, antibody targets, nucleic acids useful in gene therapy, and the like.

Clone CG52703-03 proteins include the full length protein encoded by the CG52703-03 ORF disclosed herein, as well as any mature protein arising therefrom. Such a mature protein could be formed, for example, as a result of the removal of a signal peptide. Clone CG52703-03 also includes all fragments, analogs, homologs and derivatives of Clone CG52703-03. Thus the proteins of the invention encompass both the precursors and the active forms of a protein encoded by clone CG52703-03.

### Nucleic Acids

One aspect of the invention pertains to isolated nucleic acid molecules (i.e., SEQ ID NOs:1, 3, 5, 7, 9, 11, 13, 15, 17, 19, 21, 23, 25, 27, 29, 85, 86, 123-125, 144, 147 and 149) that encode the SECX polypeptides of the invention, wherein the SECX polypeptides are selected from the group comprising those indicated in Table 1., such as clone 2191999, clone 11753149.0.6, clone 11753149.0.37, clone 3883556, clone 4301136-1, clone 4301136-2, clone 4324229, clone 4324229-2, clone AC012614_{—}1.0.123, clone 4339264-2, clone 4357764-3, clone 4391184, clone 4437909.0.4, clone 4437909.0.55, clone pCDNA3.1-TOPO-3883556-S54, clone TA4437909-S443, clone CG52703-03, clone CG52643-02, clone 259341359, clone 268824728, clone 268825987, clone 268825997, clone 275698334, clone 191998702, and clone CG52676-02. One aspect of the invention pertains to isolated polypeptides, (i.e., SEQ ID NOs:2, 4, 6, 8, 10, 12, 14, 16, 18, 20, 22, 24, 26, 28, 30, 87, 88, 126, 127, 145, 146, 148 and 150; see Table 1 and FIGS. 1-26), or biologically active portions thereof, encoded by the disclosed nucleic acids, as well as nucleic acid fragments sufficient for use as hybridization probes to identify SECX-encoding nucleic acids (e.g., SECX mRNA) and fragments for use as PCR primers for the amplification or mutation of SECX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA. SECX nucleic acids of the invention thus include SEQ ID NOs:1, 3, 5, 7, 9, 11, 13, 15, 17, 19, 21, 23, 25, 27, 29, 85, 86, 123-125, 144, 147 and 149 (Table 1 and FIGS. 1-26), and complements, fragments, homologs, and derivatives thereof.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, an isolated nucleic acid molecule encoding any one of the SECX polypeptides, including chemokine receptor-like protein, semaphorin protein-like splice variants, a putative mitochondrial protein (clone 2982339), SLIT protein-like splice variants, a putative microbody (peroxisome) associated protein (clone 3884846), a tetraspanin-like protein, a putative proline-rich membrane protein (clone 4004056), a laminin β-chain precursor-like protein, AVENA protein-like splice variants (clones 4009334-1 and 4009334-2), a fetal lung-associated protein (clone 4035508) and a myeloid upregulated protein (clone 4339264), can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., adult and fetal cells from tissues including bone tissue (including bone marrow), heart, lymph node, pancreas, spleen, thymus, placenta, kidney, liver, thalamus, brain, pituitary, breast, lung, salivary gland and adrenal gland). Moreover, an “isolated” nucleic acid molecule, e.g., a cDNA molecule, can be substantially free of other cellular material or culture medium when produced by recombinant techniques, or of chemical precursors or other chemicals when chemically synthesized.

A nucleic acid molecule of the present invention, e.g., a SECX nucleic acid molecule having the nucleotide sequence of SEQ ID NOs:1, 3, 5, 7, 9, 11, 13, 15, 17, 19, 21, 23, 25, 27, 29, 85, 86, 123-125, 144, 147 or 149, or a complement of any of these nucleotide sequences, can be isolated using standard molecular biology techniques and the sequence information provided herein. Using all or a portion of the SECX nucleic acid sequences of SEQ ID NOs:1, 3, 5, 7, 9, 11, 13, 15, 17, 19, 21, 23, 25, 27, 29, 85, 86, 123-125, 144, 147 or 149, or a complement of any of these nucleotide sequences, as a hybridization probe, said SECX molecules can be isolated using standard hybridization and cloning techniques (e.g., as described in Sambrook et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to SECX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nucleotides in length, preferably about 15 nucleotides to 30 nucleotides in length. In one embodiment, an oligonucleotide comprising a nucleic acid molecule less than 100 nucleotides in length would further comprise at lease 6 contiguous nucleotides of SEQ ID NOs:1, 3, 5, 7, 9, 11, 13, 15, 17, 19, 21, 23, 25, 27, 29, 85, 86, 123-125, 144, 147 and 149, or a complement thereof. Oligonucleotides may be chemically synthesized and may be used as probes.

In an embodiment, an isolated nucleic acid molecule of the invention comprises a SECX nucleic acid molecule that is a complement of the nucleotide sequence shown in SEQ ID NOs:1, 3, 5, 7, 9, 11, 13, 15, 17, 19, 21, 23, 25, 27, 29, 85, 86, 123-125, 144 , 147 and 149, or a portion of this nucleotide sequence. A nucleic acid molecule that is complementary to said SECX nucleotide sequences is one that is sufficiently complementary to the nucleotide sequence shown in SEQ ID NOs:1, 3, 5, 7, 9, 11, 13, 15, 17, 19, 21, 23, 25, 27, 29, 85, 86, 123-125, 144, 147 and 149,or a portion of this nucleotide sequence, that it can hydrogen bond with little or no mismatches to the given SECX nucleotide sequence, thereby forming a stable duplex.

As used herein, the term “complementary” refers to Waison-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NOs:1, 3, 5, 7, 9, 11, 13, 15, 17, 19, 21, 23, 25, 27, 29, 85, 86, 123-125, 144, 147 and 149, or the nucleotide sequence of the DNA insert of the plasmid, e.g., e.g., the pSecTag2 B and pSecV5His vectors described in Example 3, wherein e.g., a fragment that can be used as a probe or primer or a fragment encoding a biologically active portion of SECX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 30%, 50%, 70%, 80%, or 95% identity (with a preferred identity of 80-95%) over a nucleic acid or amino acid sequence of identical size or when compared to an aligned sequence in which the alignment is done by a computer homology program known in the art (e.g., see below), or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of SECX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a SECX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human SECX protein. Homologous nucleic acid sequences include those nucleic acid sequences that encode conservative amino acid substitutions (see below) in SEQ ID NOs:1, 3, 5, 7, 9, 11, 13, 15, 17, 19, 21, 23, 25, 27, 29, 85, 86, 123-125, 144, 147 and 149, as well as a polypeptide having SECX activity. Biological activities of the individual SECX proteins are described above. A homologous amino acid sequence does not encode the amino acid sequence of a human SECX polypeptide.

A SECX polypeptide is encoded by the open reading frame (“ORF”) of a SECX nucleic acid. The invention includes the nucleic acid sequence comprising the stretch of nucleic acid sequences of SEQ ID NOs:1, 3, 5, 7, 9, 11, 13, 15, 17, 19, 21, 23, 25, 27, 29, 85, 86, 123-125, 144, 147 and 149, that comprises the ORF of that nucleic acid sequence and encodes a polypeptide of SEQ ID NOs:2, 4, 6, 8, 10, 12, 14, 16, 18, 20, 22, 24, 26, 28, 30, 87, 88, 126, 127, 145, 146, 148 and 150.

An “open reading frame” (“ORF”) corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, for example, a stretch of DNA that would encode a protein of 50 amino acids or more.

The nucleotide sequence determined from the cloning of the human SECX gene allows for the generation of probes and primers designed for use in identifying and/or cloning SECX homologues in other cell types, e.g. from other tissues, as well as SECX homologues from other mammals. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOs:1, 3, 5, 7, 9, 11, 13, 15, 17, 19, 21, 23, 25, 27, 29, 85, 86, 123-125, 144, 147 and 149, or the nucleotide sequence of the DNA insert of the plasmid such as, e.g., the pSecTag2 B and pSecV5His vectors described in Example 3; or an anti-sense strand nucleotide sequence of a SECX nucleotide or the anti-sense strand SECX nucleotide sequence of the DNA insert of the plasmid known in the art; or of a naturally occurring mutant of a SECX nucleotide, or the naturally occurring mutant of the DNA insert of the plasmid vector known in the art.

Probes based on the human SECX nucleotide sequence can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissue which misexpress a SECX protein, e.g., by measuring a level of a SECX-encoding nucleic acid in a sample of cells from a subject e.g., detecting SECX mRNA levels or determining whether a genomic SECX gene has been mutated or deleted.

“A polypeptide having a biologically active portion of SECX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of SECX” can be prepared by isolating a portion of a SECX nucleotide that encodes a polypeptide having a SECX biological activity (wherein the biological activities of the SECX proteins are described above in sections 1-14), expressing the encoded portion of SECX protein (e.g., by recombinant expression in vitro) and assessing the activity of the encoded portion of SECX. For example, a nucleic acid fragment encoding a biologically active portion of SECX includes the mature polypeptide, such as amino acids 23 through 178 of clone 2191999 in SEQ ID NO:2, and as defined above by utilizing computer software programs such as PSORT and SignalP using default parameters. In another embodiment, a nucleic acid fragment encoding a biologically active portion of SECX that includes the mature polypeptide domain includes the DNA encoding such domains, e.g., at least nucleic acids 577 to 1026 of SEQ ID NO:7 that encodes the human clone 3883556 mature polypeptide domain represented by amino acid residues 17 to 166 of SEQ ID NO:8.

### SECX Variants

The invention further encompasses any one or more nucleic acid molecules that differ from the SECX nucleotide sequence shown in at least one of SEQ ID NOs:1, 3, 5, 7, 9, 11, 13, 15, 17, 19, 21, 23, 25, 27, 29, 85, 86, 123-125, 144, 147 and 149, due to degeneracy of the genetic code and thus encode the same SECX protein as that encoded by any of the above nucleotide sequences. In another embodiment, an isolated SECX nucleic acid molecule of the invention has a nucleotide sequence encoding a protein having any one amino acid sequence shown in SEQ ID NOs:2, 4, 6, 8, 10, 12, 14, 16, 18, 20, 22, 24, 26, 28, 30, 87, 88, 126, 127, 145, 146, 148 and 150.

In addition to these human SECX nucleotide sequences, or the SECX nucleotide sequence of the DNA insert of a plasmid or vector, it will be appreciated by those skilled in the art that DNA sequence polymorphisms that lead to changes in the amino acid sequences of a SECX may exist within a population (e.g., the human population). Such genetic polymorphism in a SECX gene may exist among individuals within a population due to natural allelic variation. As used herein, the terms “gene” and “recombinant gene” refer to nucleic acid molecules comprising an open reading frame encoding a SECX protein, preferably a mammalian SECX protein. Such natural allelic variations can typically result in 1-5% variance in the nucleotide sequence of the SECX gene. Any and all such nucleotide variations and resulting amino acid polymorphisms in SECX that are the result of natural allelic variation and that do not alter the functional activity of SECX are intended to be within the scope of the invention.

Moreover, nucleic acid molecules encoding SECX proteins from other species, and thus that have a nucleotide sequence that differs from the human sequence disclosed herein, are intended to be within the scope of the invention. Nucleic acid molecules corresponding to natural allelic variants and homologues of a SECX cDNAs of the invention can be isolated based on their homology to the human SECX nucleic acids disclosed herein using the human cDNAs, or a portion thereof, as a hybridization probe according to standard hybridization techniques under stringent hybridization conditions. For example, a soluble human SECX cDNA can be isolated based on its homology to human membrane-bound SECX. Likewise, a membrane-bound human SECX cDNA can be isolated based on its homology to soluble human SECX.

Allelic variants include nucleotide sequences that differ by a single nucleotide polymorphism (“SNP”). A SNP may alter the encoded amino acid sequence, as shown in clones 4437909.0.4 and 4437909.0.55, or in clones 4301136-1 and 4301136-2. Alternatively the SNP may reside in a “wobble” section of a codon in the coding region and thus remain “silent” with no alteration of the encoded polypeptide sequence, as shown in clones 4437909.0.4 and TA-4437909-S443.

Accordingly, in another embodiment, an isolated nucleic acid molecule of the invention is at least 6 nucleotides in length and hybridizes under stringent conditions to the nucleic acid molecule comprising at least one SECX nucleotide sequence. In another embodiment, the nucleic acid is at least 10, 25, 50, 100, 250, 500 or 2000 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding SECX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer oroligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a SECX nucleotide sequence corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to at least one SECX nucleic acid molecule, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known in the art. See, e.g., Ausubel et al. (eds.), 1993, CURRENT PROTOCOLS IN MOLECULAR BIOLOGY, John Wiley & Sons, NY., and Kriegler, 1990, GENE TRANSFER AND EXPRESSION, A LABORATORY MANUAL, Stockton Press, NY.

In a third embodiment, a nucleic acid that is hybridizable to at least one SECX nucleic acid molecule, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

### Conservative Mutations

In addition to naturally-occurring allelic variants of the SECX sequence that may exist in the population, the skilled artisan will further appreciate that changes can be introduced by mutation into at least one SECX nucleotide sequence of SEQ ID NOs:1, 3, 5, 7, 9, 11, 13, 15, 17, 19, 21, 23, 25, 27, 29, 85, 86, 123-125, 144, 147 and 149, thereby leading to changes in the amino acid sequence of the encoded SECX protein, without altering the functional ability of the SECX protein. For example, nucleotide substitutions leading to amino acid substitutions at “non-essential” amino acid residues can be made in the sequence of SEQ ID NOs:2, 4, 6, 8, 10, 12, 14, 16, 18, 20, 22, 24, 26, 28, 30, 87, 88, 126, 127,145, 146, 148 and 150, or the SECX nucleotide sequence of the DNA insert of the plasmid or vector known in the art. A “non-essential” amino acid residue is a residue that can be altered from the wild-type sequence of SECX without altering the biological activity, whereas an “essential” amino acid residue is required for biological activity. For example, amino acid residues that are conserved among the SECX proteins of the present invention, are predicted to be particularly unamenable to alteration.

Another aspect of the invention pertains to nucleic acid molecules encoding SECX proteins that contain changes in amino acid residues that are not essential for activity. Such SECX proteins differ in amino acid sequence from SEQ ID NOs:2, 4, 6, 8, 10, 12, 14, 16, 18, 20, 22, 24, 26, 28, 30, 87, 88, 126, 127, 145, 146, 148 and 150, yet retain biological activity. In one embodiment, the isolated nucleic acid molecule comprises a nucleotide sequence encoding a protein, wherein the protein comprises an amino acid sequence at least about 45% homologous to at least one SECX amino acid sequence. Preferably, the protein encoded by the nucleic acid molecule is at least about 60% homologous to at least one SECX polypeptide, more preferably at least about 70% homologous, at least about 80% homologous, at least about 90% homologous, and most preferably at least about 95% homologous to that given SECX polypeptide.

An isolated nucleic acid molecule encoding a SECX protein homologous to a given SECX protein can be created by introducing one or more nucleotide substitutions, additions or deletions into the corresponding SECX nucleotide sequence, such that one or more amino acid substitutions, additions or deletions are introduced into the encoded protein.

Mutations can be introduced into SEQ ID NOs:1, 3, 5, 7, 9, 11, 13, 15, 17, 19, 21, 23, 25, 27, 29, 85, 86, 123-125, 144, 147 and 149, by standard techniques, e.g., site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in SECX is replaced with another amino acid residue from the same side chain family. Alternatively, in another embodiment, mutations can be introduced randomly along all or part of a SECX coding sequence, e.g., by saturation mutagenesis, and the resultant mutants can be screened for SECX biological activity to identify mutants that retain activity. Following mutagenesis, the encoded SECX protein can be expressed by any recombinant technology known in the art and the activity of the protein can be determined.

In one embodiment, a mutant SECX protein can be assayed for (1) the ability to form protein:protein interactions with other SECX proteins, other cell-surface proteins, or biologically active portions thereof, (2) complex formation between a mutant SECX protein and a SECX ligand; (3) the ability of a mutant SECX protein to bind to an intracellular target protein or biologically active portion thereof; (e.g. avidin proteins).

### Antisense

Another aspect of the invention pertains to isolated antisense nucleic acid molecules that are hybridizable to or complementary to a SECX nucleic acid molecule, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire SECX coding strand, or to only a portion thereof. Nucleic acid molecules encoding fragments, homologs, derivatives and analogs of a SECX protein, or antisense nucleic acids complementary to a SECX nucleic acid sequence, are additionally provided.

In one embodiment, an antisense nucleic acid molecule is antisense to a “coding region” of the coding strand of a nucleotide sequence encoding SECX. The term “coding region” refers to the region of the nucleotide sequence comprising codons which are translated into amino acid residues (e.g., ORFs shown in FIGS. 1-14 and 18-19). In another embodiment, the antisense nucleic acid molecule is antisense to a “noncoding region” of the coding strand of a nucleotide sequence encoding SECX. The term “noncoding region” refers to 5′ and 3′ sequences which flank the coding region that are not translated into amino acids (i.e., also referred to as 5′ and 3′ untranslated regions).

Given the coding strand sequences encoding SECX disclosed herein (e.g., SEQ ID NOs:1, 3, 5, 7, 9, 11, 13, 15, 17, 19, 21, 23, 25, 27, 29, 85, 86, 123-125, 144, 147 and 149), antisense nucleic acids of the invention can be designed according to the rules of Watson and Crick or Hoogsteen base pairing. The antisense nucleic acid molecule can be complementary to the entire coding region of SECX mRNA, but more preferably is an oligonucleotide that is antisense to only a portion of the coding or noncoding region of SECX mRNA. For example, the antisense oligonucleotide can be complementary to the region surrounding the translation start site of SECX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, dihydrouracil, inosine, 5-carboxymethylaminomethyluracil, beta-D-galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a SECX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

### Ribozymes and PNA Moieties

Nucleic acid modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, e.g., an mRNA, to which they have a complementary region. Thus, ribozymes (e.g., hammerhead ribozymes (described in Haselhoff and Gerlach (1988) Nature 334:585-591)) can be used to catalytically cleave SECX mRNA transcripts to thereby inhibit translation of SECX mRNA. A ribozyme having specificity for a SECX-encoding nucleic acid can be designed based upon the nucleotide sequence of a SECX cDNA disclosed herein (i.e., SEQ ID NOs:1, 3, 5, 7, 9, 11, 13, 15, 17, 19, 21, 23, 25, 27, 29, 85, 86, 123-125, 144, 147 and 149). For example, a derivative of a Tetrahymena L-19 IVS RNA can be constructed in which the nucleotide sequence of the active site is complementary to the nucleotide sequence to be cleaved in a SECX-encoding mRNA. See, e.g., Cech et al. U.S. Pat. No. 4,987,071; and Cech et al. U.S. Pat. No. 5,116,742. Alternatively, SECX mRNA can be used to select a catalytic RNA having a specific ribonuclease activity from a pool of RNA molecules. See, e.g., Bartel et al., (1993) Science 261:1411-1418.

Alternatively, SECX gene expression can be inhibited by targeting nucleotide sequences complementary to the regulatory region of a SECX gene (e.g., the SECX promoter and/or enhancers) to form triple helical structures that prevent transcription of the SECX gene in target cells. See generally, Helene. (1991) Anticancer Drug Des. 6: 569-84; Helene. et al. (1992) Ann. N. Y. Acad. Sci. 660:27-36; and Maher (1992) Bioassays 14: 807-15.

In various embodiments, the nucleic acids of SECX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of SECX can be used in therapeutic and diagnostic applications. For example, PNAs can be used as antisense or antigene agents for sequence-specific modulation of gene expression by, e.g., inducing transcription or translation arrest or inhibiting replication. PNAs of SECX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of SECX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of SECX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3 ′ PNA segment. See, Petersen et al. (1975) Bioorg Med Chem Lett5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

### SECX Proteins

The novel protein of the invention includes the SECX proteins whose sequences are provided in FIGS. 1-14 and 18-19 (SEQ ID NOs:2, 4, 6, 8, 10, 12, 14, 16, 18, 20, 22, 24, 26, 28, 30, 87, 88, 126, 127, 145, 146, 148 and 150). The invention also includes a mutant or variant protein, any of whose residues may be changed from the corresponding residue shown in FIGS. 1-14 and 18-19 while still encoding a protein that maintains its SECX activities and physiological functions, or a functional fragment thereof. In the mutant or variant protein, up to 20% or more of the residues may be so changed.

In general, an SECX variant that preserves SECX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated SECX proteins, and biologically active portions thereof, or derivatives, fragments, analogs or homologs thereof. Also provided are polypeptide fragments suitable for use as immunogens to raise anti-SECX antibodies. In one embodiment, native SECX proteins can be isolated from cells or tissue sources by an appropriate purification scheme using standard protein purification techniques. In another embodiment, SECX proteins are produced by recombinant DNA techniques. Alternative to recombinant expression, a SECX protein or polypeptide can be synthesized chemically using standard peptide synthesis techniques.

An “isolated” or “purified” protein or biologically active portion thereof is substantially free of cellular material or other contaminating proteins from the cell or tissue source from which the SECX protein is derived, or substantially free from chemical precursors or other chemicals when chemically synthesized. The language “substantially free of cellular material” includes preparations of SECX protein in which the protein is separated from cellular components of the cells from which it is isolated or recombinantly produced. In one embodiment, the language “substantially free of cellular material” includes preparations of SECX protein having less than about 30% (by dry weight) of non-SECX protein (also referred to herein as a “contaminating protein”), more preferably less than about 20% of non-SECX protein, still more preferably less than about 10% of non-SECX protein, and most preferably less than about 5% non-SECX protein. When the SECX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of SECX protein in which the protein is separated from chemical precursors or other chemicals that are involved in the synthesis of the protein. In one embodiment, the language “substantially free of chemical precursors or other chemicals” includes preparations of SECX protein having less than about 30% (by dry weight) of chemical precursors or non-SECX chemicals, more preferably less than about 20% chemical precursors or non-SECX chemicals, still more preferably less than about 10% chemical precursors or non-SECX chemicals, and most preferably less than about 5% chemical precursors or non-SECX chemicals.

Biologically active portions of a SECX protein include peptides comprising amino acid sequences sufficiently homologous to or derived from the amino acid sequence of the SECX protein, e.g., the amino acid sequence shown in SEQ ID NOs:2, 4, 6, 8, 10, 12, 14, 16, 18, 20, 22, 24, 26, 28, 30, 87, 88, 126, 127, 145, 146, 148 and 150, that include fewer amino acids than the full length SECX proteins, and exhibit at least one activity of a SECX protein. Typically, biologically active portions comprise a domain or motif with at least one activity of the SECX protein. A biologically active portion of a SECX protein can be a polypeptide which is, for example, 10, 25, 50, 100 or more amino acids in length.

It is to be understood that a biologically active portion of a SECX protein of the present invention may contain at least one of the structural domains identified in Sections 1-14, above. An alternative biologically active portion of a SECX protein may contain an extracellular domain of the SECX protein. Another biologically active portion of a SECX protein may contain the transmembrane domain of the SECX protein. Yet another biologically active portion of a SECX protein of the present invention may contain the intracellular domain of the SECX protein.

Moreover, other biologically active portions, in which other regions of the protein are deleted, can be prepared by recombinant techniques and evaluated for one or more of the functional activities of a native SECX protein.

In an embodiment, the SECX protein has any one or more amino acid sequences shown in SEQ ID NOs:2, 4, 6, 8, 10, 12, 14, 16, 18, 20, 22, 24, 26, 28, 30, 87, 88, 126, 127, 145, 146, 148 and 150. In other embodiments, the SECX protein is substantially homologous to any one of SEQ ID NOs:2, 4, 6, 8, 10, 12, 14, 16, 18, 20, 22, 24, 26, 28, 30, 87, 88, 126, 127, 145, 146, 148 and 150, and retains the functional activity of that given SECX protein yet differs in amino acid sequence due to natural allelic variation or mutagenesis, as described in detail below. Accordingly, in another embodiment, the SECX protein is a protein that comprises an amino acid sequence at least about 75% homologous to any one amino acid sequence of SEQ ID NOs:2, 4, 6, 8, 10, 12, 14, 16, 18, 20, 22, 24, 26, 28, 30, 87, 88, 126, 127, 145, 146, 148 and 150, and retains the functional activity of that SECX protein.

This invention further features isolated SECX protein, or derivatives, fragments, analogs or homologs thereof, that is encoded by a nucleic acid molecule having a nucleotide sequence that hybridizes under stringent hybridization conditions to a nucleic acid molecule comprising the nucleotide sequence of any one or more of SEQ ID NOs:1, 3, 5, 7, 9, 11, 13, 15, 17, 19, 21, 23, 25, 27, 29, 85, 86, 123-125, 144, 147 and 149.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (i.e., encoding) part of the DNA sequence shown in any one or more of SEQ ID NOs:1, 3, 5, 7, 9, 11, 13, 15, 17, 19, 21, 23, 25, 27, 29, 85, 86, 123-125, 144, 147 and 149.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to. 95 percent sequence identity, more usually at least 99 percent sequence identity as compared to a reference sequence over a comparison region. Similar calculation are used when comparing amino acid residues in polypeptide sequences.

### Chimeric and Fusion Proteins

The invention also provides SECX chimeric or fusion proteins. As used herein, a SECX “chimeric protein” or “fusion protein” comprises a SECX polypeptide operatively linked to a non-SECX polypeptide. A “SECX polypeptide” refers to a polypeptide having an amino acid sequence corresponding to SECX, whereas a “non-SECX polypeptide” refers to a polypeptide having an amino acid sequence corresponding to a protein that is not substantially homologous to the SECX protein, e.g., a protein that is different from the SECX protein and that is derived from the same or a different organism. Within a SECX fusion protein the SECX polypeptide can correspond to all or a portion of a SECX protein. In one embodiment, a SECX fusion protein comprises at least one biologically active portion of a SECX protein. In another embodiment, a SECX fusion protein comprises at least two biologically active portions of a SECX protein. In yet another embodiment, a SECX fusion protein comprises at least three biologically active portions of a SECX protein. Within the fusion protein, the term “operatively linked” is intended to indicate that the SECX polypeptide and the non-SECX polypeptide are fused in-frame to each other. The non-SECX polypeptide can be fused to the N-terminus or C-terminus of the SECX polypeptide.

For example, in one embodiment a SECX fusion protein comprises a SECX domain operably linked to the extracellular domain of a second protein known to be involved in an activity of interest. Such fusion proteins can be further utilized in screening assays for compounds which modulate SECX activity (such assays are described in detail below).

In one embodiment, the fusion protein is a GST-SECX fusion protein in which the SECX sequences are fused to the C-terminus of the GST (i.e., glutathione S-transferase) sequences. Such fusion proteins can facilitate the purification of recombinant SECX.

In another embodiment, the fusion protein is a SECX protein containing a heterologous signal sequence at its N-terminus. For example, the native SECX signal sequence (i.e., about amino acids 1 to 26, or as described in Sections 1-14 above) can be removed and replaced with a signal sequence from another protein. In certain host cells (e.g., mammalian host cells), expression and/or secretion of SECX can be increased through use of a heterologous signal sequence.

In yet another embodiment, the fusion protein is a SECX-immunoglobulin fusion protein in which the SECX sequences comprising primarily the extracellular domains are fused to sequences derived from a member of the immunoglobulin protein family. The SECX-immunoglobulin fusion proteins of the invention can be incorporated into pharmaceutical compositions and administered to a subject to inhibit an interaction between a SECX ligand and a SECX protein on the surface of a cell, to thereby suppress SECX-mediated signal transduction in vivo. The SECX-immunoglobulin fusion proteins can be used to affect the bioavailability of a SECX cognate ligand. Inhibition of the SECX ligand/SECX interaction may be useful therapeutically for both the treatment of proliferative and differentiative disorders, as well as modulating (e.g. promoting or inhibiting) cell survival. Moreover, the SECX-immunoglobulin fusion proteins of the invention can be used as immunogens to produce anti-SECX antibodies in a subject, to purify SECX ligands, and in screening assays to identify molecules that inhibit the interaction of SECX with a SECX ligand.

A SECX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A SECX-encoding nucleic acid can be cloned into such an expression vector such that the fusion moiety is linked in-frame to the SECX protein.

The invention also provides signal sequences derived from various SECX polypeptides. The signal sequences include, e.g., polypeptides including the signal peptides identified for the SECX polypeptides as predicted by the SignalP software program for the SECX polypeptides described above. These signal sequences are useful for directing a linked polypeptide sequence to a desired intracellular or extracellular (if secretion from the cell is desired) location. In some embodiments, the signal sequence includes a portion of a SECX signal sequence that is sufficient to direct a linked polypeptide to a desired cellular compartment.

### SECX Agonists and Antagonists

The present invention also pertains to variants of the SECX proteins that function as either SECX agonists (mimetics) or as SECX antagonists. Variants of the SECX protein can be generated by mutagenesis, e.g., discrete point mutation or truncation of the SECX protein. An agonist of the SECX protein can retain substantially the same, or a subset of, the biological activities of the naturally occurring form of the SECX protein. An antagonist of the SECX protein can inhibit one or more of the activities of the naturally occurring form of the SECX protein by, for example, competitively binding to a downstream or upstream member of a cellular signaling cascade which includes the SECX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the SECX proteins.

Variants of the SECX protein that function as either SECX agonists (mimetics) or as SECX antagonists can be identified by screening combinatorial libraries of mutants, e.g., truncation mutants, of the SECX protein for SECX protein agonist or antagonist activity. In one embodiment, a variegated library of SECX variants is generated by combinatorial mutagenesis at the nucleic acid level and is encoded by a variegated gene library. A variegated library of SECX variants can be produced by, for example, enzymatically ligating a mixture of synthetic oligonucleotides into gene sequences such that a degenerate set of potential SECX sequences is expressible as individual polypeptides, or alternatively, as a set of larger fusion proteins (e.g., for phage display) containing the set of SECX sequences therein. There are a variety of methods which can be used to produce libraries of potential SECX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SECX sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

### Polypeptide Libraries

In addition, libraries of fragments of the SECX protein coding sequence can be used to generate a variegated population of SECX fragments for screening and subsequent selection of variants of a SECX protein. In one embodiment, a library of coding sequence fragments can be generated by treating a double stranded PCR fragment of a SECX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the SECX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SECX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique that enhances the frequency of functional mutants in the libraries, can be used in combination with the screening assays to identify SECX variants (Arkin and Yourvan (1992) PNAS 89:7811-7815; Delgrave et al. (1993) Protein Engineering 6:327-331).

In one embodiment, cell based assays can be exploited to analyze a variegated SECX library, e.g., a library of mutant SECX polypeptides. For example, a library of expression vectors can be transfected into a cell line that ordinarily responds to a particular ligand or receptor in a SECX-dependent manner, e.g., through a signaling complex. The transfected cells are then contacted with the putative SECX interactant and the effect of expression of the mutant SECX on signaling by the signaling complex can be detected, e.g. by measuring a cellular activity or cell survival. Plasmid DNA can then be recovered from the cells which score for inhibition, or alternatively, potentiation of, e.g., cytokine induction, and the individual clones further characterized.

### Anti-SECX Antibodies

The invention encompasses antibodies and antibody fragments, such as F_{ab} or (F_{ab})_{2,} that bind immunospecifically to any of the polypeptides of the invention.

An isolated SECX protein, or a portion or fragment thereof, can be used as an immunogen to generate antibodies that bind SECX using standard techniques for polyclonal and monoclonal antibody preparation. The full-length SECX protein can be used or, alternatively, the invention provides antigenic peptide fragments of SECX for use as immunogens. The antigenic peptide of SECX comprises at least 4 amino acid residues of the amino acid sequence shown in SEQ ID NOs:2, 4, 6, 8, 10, 12, 14, 16, 18, 20, 22, 24, 26, 28, 30, 87, 88, 126, 127, 145, 146, 148 and 150 and encompasses an epitope of SECX such that an antibody raised against the peptide forms a specific immune complex with SECX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of SECX that is located on the surface of the protein, e.g., a hydrophilic region. A hydrophobicity analysis of the human SECX protein sequence will indicate which regions of a SECX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety.

As disclosed herein, SECX protein sequence of SEQ ID NOs:2, 4, 6, 8, 10, 12, 14, 16, 18, 20, 22, 24, 26, 28, 30, 87, 88, 126, 127, 145, 146, 148 and 150, or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen, such as SECX. Such antibodies include, but are not limited to, polyclonal, monoclonal, chimeric, single chain, F_{ab} and F_{(ab′)2} fragments, and an F_{ab} expression library. In a specific embodiment, antibodies to human SECX proteins are disclosed. Various procedures known within the art may be used for the production of polyclonal or monoclonal antibodies to a SECX protein sequence, or derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed SECX protein or a chemically synthesized SECX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against SECX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of SECX. A monoclonal antibody composition thus typically displays a single binding affinity for a particular SECX protein with which it immunoreacts. For preparation of monoclonal antibodies directed towards a particular SECX protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96). Each of the above citations are incorporated herein by reference in their entirety.

According to the invention, techniques can be adapted for the production of single-chain antibodies specific to a SECX protein (see e.g., U.S. Pat. No.4,946,778). In addition, methodologies can be adapted for the construction of F_{ab} expression libraries (see e.g., Huse, et al., 1989 Science 246: 1275-1281) to allow rapid and effective identification of monoclonal F_{ab} fragments with the desired specificity for a SECX protein or derivatives, fragments, analogs or homologs thereof. Non-human antibodies can be “humanized” by techniques well known in the art. See e.g., U.S. Pat. No. 5,225,539. Antibody fragments that contain the idiotypes to a SECX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-SECX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No.184,187; European Patent Application No. 171,496; European Patent Application No. 173,494; PCT International Publication No. WO 86/01533; U.S. Pat. No. 4,816,567; U.S. Pat. No.5,225,539; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst 80:1553-1559); Morrison(1985) Science 229:1202-1207; Oi et al. (1986) BioTechniques 4:214; Jones et al. (1986) Nature 321:552-525; Verhoeyan et al. (1988) Science 239:1534; and Beidler et al. (1988) J Immunol 141:4053-4060. Each of the above citations are incorporated herein by reference in their entirety.

In one embodiment, methodologies for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a SECX protein is facilitated by generation of hybridomas that bind to the fragment of a SECX protein possessing such a domain. Antibodies that are specific for an above-described domain within a SECX protein, or derivatives, fragments, analogs or homologs thereof, are also provided herein.

Anti-SECX antibodies may be used in methods known within the art relating to the localization and/or quantitation of a SECX protein (e.g., for use in measuring levels of the SECX protein within appropriate physiological samples, for use in diagnostic methods, for use in imaging the protein, and the like). In a given embodiment, antibodies for SECX proteins, or derivatives, fragments, analogs or homologs thereof, that contain the antibody derived binding domain, are utilized as pharmacologically-active compounds [hereinafter “Therapeutics”].

An anti-SECX antibody (e.g., monoclonal antibody) can be used to isolate SECX by standard techniques, such as affinity chromatography or immunoprecipitation. An anti-SECX antibody can facilitate the purification of natural SECX from cells and of recombinantly produced SECX expressed in host cells. Moreover, an anti-SECX antibody can be used to detect SECX protein (e.g., in a cellular lysate or cell supernatant) in order to evaluate the abundance and pattern of expression of the SECX protein. Anti-SECX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### SECX Recombinant Expression Vectors and Host Cells

Another aspect of the invention pertains to vectors, preferably expression vectors, containing a nucleic acid encoding SECX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., SECX proteins, mutant forms of SECX, fusion proteins, etc.).

The recombinant expression vectors of the invention can be designed for expression of SECX in prokaryotic or eukaryotic cells. For example, SECX can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc.; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the SECX expression vector is a yeast expression vector. Examples of vectors for expression in yeast S. cerivisae include pYepSec1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp., San Diego, Calif.).

Alternatively, SECX can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to SECX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, SECX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding SECX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) SECX protein. Accordingly, the invention further provides methods for producing SECX protein using the host cells of the invention. In one embodiment, the method comprises culturing the host cell of invention (into which a recombinant expression vector encoding SECX has been introduced) in a suitable medium such that SECX protein is produced. In another embodiment, the method further comprises isolating SECX from the medium or the host cell.

### Transgenic Animals

The host cells of the invention can also be used to produce nonhuman transgenic animals. For example, in one embodiment, a host cell of the invention is a fertilized oocyte or an embryonic stem cell into which SECX-coding sequences have been introduced. Such host cells can then be used to create non-human transgenic animals in which exogenous SECX sequences have been introduced into their genome or homologous recombinant animals in which endogenous SECX sequences have been altered. Such animals are useful for studying the function and/or activity of SECX and for identifying and/or evaluating modulators of SECX activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous SECX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing SECX-encoding nucleic acid into the male pronuclei of a fertilized oocyte, e.g., by microinjection, retroviral infection, and allowing the oocyte to develop in a pseudopregnant female foster animal. The human SECX cDNA can be introduced as a transgene into the genome of a non-human animal. Alternatively, a nonhuman homologue of the human SECX gene, such as a mouse SECX gene, can be isolated based on hybridization to the human SECX cDNA (described further above) and used as a transgene. Intronic sequences and polyadenylation signals can also be included in the transgene to increase the efficiency of expression of the transgene. A tissue-specific regulatory sequence(s) can be operably linked to the SECX transgene to direct expression of SECX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the SECX transgene in its genome and/or expression of SECX mRNA in tissues or cells of the animals. A transgenic founder animal can then be used to breed additional animals carrying the transgene. Moreover, transgenic animals carrying a transgene encoding SECX can further be bred to other transgenic animals carrying other transgenes.

To create a homologous recombinant animal, a vector is prepared which contains at least a portion of a SECX gene into which a deletion, addition or substitution has been introduced to thereby alter, e.g., functionally disrupt, the SECX gene. The SECX gene can be a human gene (e.g., the cDNA of SEQ ID NOs:1, 3, 5, 7, 9, 11, 13, 15, 17, 19, 21, 23, 25, 27, 29, 85, 86, 123-125, 144, 147 and 149), but more preferably, is a non-human homologue of a human SECX gene. For example, a mouse homologue of human SECX gene of, e.g., SEQ ID NO :29, can be used to construct a homologous recombination vector suitable for altering an endogenous SECX gene in the mouse genome. In one embodiment, the vector is designed such that, upon homologous recombination, the endogenous SECX gene is functionally disrupted (i.e., no longer encodes a functional protein; also referred to as a “knock out” vector).

Alternatively, the vector can be designed such that, upon homologous recombination, the endogenous SECX gene is mutated or otherwise altered but still encodes functional protein (e.g., the upstream regulatory region can be altered to thereby alter the expression of the endogenous SECX protein). In the homologous recombination vector, the altered portion of the SECX gene is flanked at its 5′ and 3′ ends by additional nucleic acid of the SECX gene to allow for homologous recombination to occur between the exogenous SECX gene carried by the vector and an endogenous SECX gene in an embryonic stem cell. The additional flanking SECX nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51:503 for a description of homologous recombination vectors. The vector is introduced into an embryonic stem cell line (e.g., by electroporation) and cells in which the introduced SECX gene has homologously recombined with the endogenous SECX gene are selected (see e.g., Li et al. (1992) Cell 69:915).

The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2:823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

### Pharmaceutical Compositions

The SECX nucleic acid molecules, SECX proteins, and anti-SECX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a SECX protein or anti-SECX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No.5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### USES AND METHODS OF THE INVENTION

The nucleic acid molecules, proteins, protein homologues, and antibodies described herein that include extracellular and transmembrane domains and, therefore, can be used in one or more of the following methods: (a) screening assays; (b) detection assays (e.g., chromosomal mapping, tissue typing, forensic biology), (c) predictive medicine (e.g., diagnostic assays, prognostic assays, monitoring clinical trials, and pharmacogenomics); and (d) methods of treatment (e.g., therapeutic and prophylactic). A SECX protein interacting with other cellular proteins can thus be used to (i) modulate that respective protein activity; (ii) regulate cellular proliferation; (iii) regulate cellular differentiation; and (iv) regulate cell survival.

The isolated nucleic acid molecules of the invention can be used to express SECX protein (e.g., via a recombinant expression vector in a host cell in gene therapy applications), to detect SECX mRNA (e.g., in a biological sample) or a genetic lesion in a SECX gene, and to modulate SECX activity, as described further below. In addition, the SECX proteins can be used to screen drugs or compounds that modulate the SECX activity or expression as well as to treat disorders characterized by insufficient or excessive production of SECX protein or production of SECX protein forms that have decreased or aberrant activity compared to SECX wild type protein (e.g. proliferative disorders such as cancer or preclampsia, or any disease or disorder described in Sections 1-14 above). In addition, the anti-SECX antibodies of the invention can be used to detect and isolate SECX proteins and modulate SECX activity.

This invention further pertains to novel agents identified by the above described screening assays and uses thereof for treatments as described herein.

### Screening Assays

The invention provides a method (also referred to herein as a “screening assay”) for identifying modulators, i.e., candidate or test compounds or agents (e.g., peptides, peptidomimetics, small molecules or other drugs) that bind to SECX proteins or have a stimulatory or inhibitory effect on, for example, SECX expression or SECX activity.

In one embodiment, the invention provides assays for screening candidate or test compounds which bind to or modulate the activity of the membrane-bound form of a SECX protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) BioTechniques 13:412421), or on beads (Lam (1991) Nature 354:82-84), on chips (Fodor (1993) Nature 364:555-556), bacteria (Ladner U.S. Pat. No.5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404406; Cwirla et al. (1990) Proc Natl Acad Sci U.S.A. 87:6378-6382; Felici (1991) J Mol Biol 222:301-310; Ladner above.).

In one embodiment, an assay is a cell-based assay in which a cell which expresses a membrane-bound form of SECX protein, or a biologically active portion thereof, on the cell surface is contacted with a test compound and the ability of the test compound to bind to a SECX protein determined. The cell, for example, can of mammalian origin or a yeast cell. Determining the ability of the test compound to bind to the SECX protein can be accomplished, for example, by coupling the test compound with a radioisotope or enzymatic label such that binding of the test compound to the SECX protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of SECX protein, or a biologically active portion thereof, on the cell surface with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the test compound to preferentially bind to SECX or a biologically active portion thereof as compared to the known compound.

In another embodiment, an assay is a cell-based assay comprising contacting a cell expressing a membrane-bound form of SECX protein, or a biologically active portion thereof, on the cell surface with a test compound and determining the ability of the test compound to modulate (e.g., stimulate or inhibit) the activity of the SECX protein or biologically active portion thereof. Determining the ability of the test compound to modulate the activity of SECX or a biologically active portion thereof can be accomplished, for example, by determining the ability of the SECX protein to bind to or interact with a SECX target molecule. As used herein, a “target molecule” is a molecule with which a SECX protein binds or interacts in nature, for example, a molecule on the surface of a cell which expresses a SECX interacting protein, a molecule on the surface of a second cell, a molecule in the extracellular milieu, a molecule associated with the internal surface of a cell membrane or a cytoplasmic molecule. A SECX target molecule can be a non-SECX molecule or a SECX protein or polypeptide of the present invention. In one embodiment, a SECX target molecule is a component of a signal transduction pathway that facilitates transduction of an extracellular signal (e.g. a signal generated by binding of a compound to a membrane-bound SECX molecule) through the cell membrane and into the cell. The target, for example, can be a second intercellular protein that has catalytic activity or a protein that facilitates the association of downstream signaling molecules with SECX.

Determining the ability of the SECX protein to bind to or interact with a SECX target molecule can be accomplished by one of the methods described above for determining direct binding. In one embodiment, determining the ability of the SECX protein to bind to or interact with a SECX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a SECX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a SECX protein or biologically active portion thereof with a test compound and determining the ability of the test compound to bind to the SECX protein or biologically active portion thereof. Binding of the test compound to the SECX protein can be determined either directly or indirectly as described above. In one embodiment, the assay comprises contacting the SECX protein or biologically active portion thereof with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the test compound to preferentially bind to SECX or biologically active portion thereof as compared to the known compound.

In another embodiment, an assay is a cell-free assay comprising contacting SECX protein or biologically active portion thereof with a test compound and determining the ability of the test compound to modulate (e.g. stimulate or inhibit) the activity of the SECX protein or biologically active portion thereof. Determining the ability of the test compound to modulate the activity of SECX can be accomplished, for example, by determining the ability of the SECX protein to bind to a SECX target molecule by one of the methods described above for determining direct binding. In an alternative embodiment, determining the ability of the test compound to modulate the activity of SECX can be accomplished by determining the ability of the SECX protein further modulate a SECX target molecule. For example, the catalytic/enzymatic activity of the target molecule on an appropriate substrate can be determined as previously described.

In yet another embodiment, the cell-free assay comprises contacting the SECX protein or biologically active portion thereof with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the SECX protein to preferentially bind to or modulate the activity of a SECX target molecule.

The cell-free assays of the present invention are amenable to use of both the soluble form or the membrane-bound form of SECX. In the case of cell-free assays comprising the membrane-bound form of SECX, it may be desirable to utilize a solubilizing agent such that the membrane-bound form of SECX is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl) dimethylamminiol-2-hydroxy-1-propane sulfonate (CHAPSO).

In more than one embodiment of the above assay methods of the present invention, it may be desirable to immobilize either SECX or its target molecule to facilitate separation of complexed from uncomplexed forms of one or both of the proteins, as well as to accommodate automation of the assay. Binding of a test compound to SECX, or interaction of SECX with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-SECX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or SECX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of SECX binding or activity determined using standard techniques.

Other techniques for immobilizing proteins on matrices can also be used in the screening assays of the invention. For example, either SECX or its target molecule can be immobilized utilizing conjugation of biotin and streptavidin. Biotinylated SECX or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with SECX or target molecules, but which do not interfere with binding of the SECX protein to its target molecule, can be derivatized to the wells of the plate, and unbound target or SECX trapped in the wells by antibody conjugation. Methods for detecting such complexes, in addition to those described above for the GST-immobilized complexes, include immunodetection of complexes using antibodies reactive with the SECX or target molecule, as well as enzyme-linked assays that rely on detecting an enzymatic activity associated with the SECX or target molecule.

In another embodiment, modulators of SECX expression are identified in a method wherein a cell is contacted with a candidate compound and the expression of SECX mRNA or protein in the cell is determined. The level of expression of SECX mRNA or protein in the presence of the candidate compound is compared to the level of expression of SECX mRNA or protein in the absence of the candidate compound. The candidate compound can then be identified as a modulator of SECX expression based on this comparison. For example, when expression of SECX mRNA or protein is greater (statistically significantly greater) in the presence of the candidate compound than in its absence, the candidate compound is identified as a stimulator of SECX mRNA or protein expression. Alternatively, when expression of SECX mRNA or protein is less (statistically significantly less) in the presence of the candidate compound than in its absence, the candidate compound is identified as an inhibitor of SECX mRNA or protein expression. The level of SECX mRNA or protein expression in the cells can be determined by methods described herein for detecting SECX mRNA or protein.

In yet another aspect of the invention, the SECX proteins can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) BioTechniques 14:920-924; Iwabuchi et al. (1993) Oncogene 8:1693-1696; and Brent WO94/10300), to identify other proteins that bind to or interact with SECX (“SECX-binding proteins” or “SECX-bp”) and modulate SECX activity. Such SECX-binding proteins are also likely to be involved in the propagation of signals by the SECX proteins as, for example, upstream or downstream elements of the SECX pathway.

The two-hybrid system is based on the modular nature of most transcription factors, which consist of separable DNA-binding and activation domains. Briefly, the assay utilizes two different DNA constructs. In one construct, the gene that codes for SECX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a SECX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with SECX.

This invention further pertains to novel agents identified by the above-described screening assays and uses thereof for treatments as described herein.

### Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

### Chromosome Mapping

Once the sequence (or a portion of the sequence) of a gene has been isolated, this sequence can be used to map the location of the gene on a chromosome. This process is called chromosome mapping. Accordingly, portions or fragments of the SECX, sequences, described herein, can be used to map the location of the SECX genes, respectively, on a chromosome. The mapping of the SECX sequences to chromosomes is an important first step in correlating these sequences with genes associated with disease.

Briefly, SECX genes can be mapped to chromosomes by preparing PCR primers (preferably 15-25 bp in length) from the SECX sequences. Computer analysis of the SECX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the SECX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the SECX sequences to design oligonucleotide primers, sublocalization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see Verma et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, for example, in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, for example, Egeland et al. (1987) Nature, 325:783-787.

Moreover, differences in the DNA sequences between individuals affected and unaffected with a disease associated with the SECX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

The SECX sequences of the present invention can also be used to identify individuals from minute biological samples. In this technique, an individual's genomic DNA is digested with one or more restriction enzymes, and probed on a Southern blot to yield unique bands for identification. The sequences of the present invention are useful as additional DNA markers for RFLP (“restriction fragment length polymorphisms,” described in U.S. Pat. No. 5,272,057).

Furthermore, the sequences of the present invention can be used to provide an alternative technique that determines the actual base-by-base DNA sequence of selected portions of an individual's genome. Thus, the SECX sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The SECX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can be used as a standard against which DNA from an individual can be compared for identification purposes. Because greater numbers of polymorphisms occur in the noncoding regions, fewer sequences are necessary to differentiate individuals. The noncoding sequences of any one or more of SEQ ID NOs:1, 3, 5, 7, 9, 11, 13, 15, 17, 19, 21, 23, 25, 27, 29, 85, 86, 123-125, 144, 147 and 149 can comfortably provide positive individual identification with a panel of perhaps 10 to 1,000 primers that each yield a noncoding amplified sequence of 100 bases. If predicted coding sequences are used, a more appropriate number of primers for positive individual identification would be 500-2,000.

A further use of the SECX sequences is to identify a cell or tissue type in a biological sample. As discussed above, various SECX genes are expressed in one or more cell types. Thus, a cell type can be identified based on the presence of RNA molecules from one or more SECX genes. Tissue distribution of various SECX genes are shown and discussed in FIGS. 20-23 and Examples 8-11, below.

### Use of SECX Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, that can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SECX gene are particularly appropriate for this use, as greater numbers of polymorphisms occur in the noncoding regions, making it easier to differentiate individuals using this technique. Examples of polynucleotide reagents include the SECX sequences or portions thereof, e.g., fragments derived from the noncoding regions of a SECX gene described herein, having a length of at least 20 bases, preferably at least 30 bases.

The SECX sequences described herein can further be used to provide polynucleotide reagents, e.g., labeled or labelable probes that can be used, for example, in an in situ hybridization technique, to identify a specific tissue, e.g., brain tissue, etc. This can be very useful in cases where a forensic pathologist is presented with a tissue of unknown origin. Panels of such SECX probes can be used to identify tissue by species and/or by organ type.

In a similar fashion, these reagents, e.g., SECX primers or probes can be used to screen tissue culture for contamination (i.e. screen for the presence of a mixture of different types of cells in a culture).

### Determination of the Biological Effect of the Therapeutic

In various embodiments of the present invention, suitable in vitro or in vivo assays are utilized to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Malignancies

SECX proteins are located at the cellular membrane and are thought to be involved in the regulation of cell proliferation and differentiation. Accordingly, Therapeutics of the present invention may be useful in the therapeutic or prophylactic treatment of diseases or disorders that are associated with cell hyperproliferation and/or loss of control of cell proliferation (e.g., cancers, malignancies and tumors). For a review of such hyperproliferation disorders, see e.g., Fishman, et al., 1985. MEDICINE, 2nd ed., J.B. Lippincott Co., Philadelphia, Pa.

Therapeutics of the present invention may be assayed by any method known within the art for efficacy in treating or preventing malignancies and related disorders. Such assays include, but are not limited to, in vitro assays utilizing transformed cells or cells derived from the patient's tumor, as well as in vivo assays using animal models of cancer or malignancies. Potentially effective Therapeutics are those that, for example, inhibit the proliferation of tumor-derived or transformed cells in culture or cause a regression of tumors in animal models, in comparison to the controls.

In the practice of the present invention, once a malignancy or cancer has been shown to be amenable to treatment by modulating (i.e., inhibiting, antagonizing or agonizing) activity, that cancer or malignancy may subsequently be treated or prevented by the administration of a Therapeutic that serves to modulate protein function.

### Premalignant Conditions

The Therapeutics of the present invention that are effective in the therapeutic or prophylactic treatment of cancer or malignancies may also be administered for the treatment of pre-malignant conditions and/or to prevent the progression of a pre-malignancy to a neoplastic or malignant state. Such prophylactic or therapeutic use is indicated in conditions known or suspected of preceding progression to neoplasia or cancer, in particular, where non-neoplastic cell growth consisting of hyperplasia, metaplasia or, most particularly, dysplasia has occurred. For a review of such abnormal cell growth see e.g., Robbins & Angell, 1976. BASIC PATHOLOGY, 2nd ed., W.B. Saunders Co., Philadelphia, Pa.

Hyperplasia is a form of controlled cell proliferation involving an increase in cell number in a tissue or organ, without significant alteration in its structure or function. For example, it has been demonstrated that endometrial hyperplasia often precedes endometrial cancer. Metaplasia is a form of controlled cell growth in which one type of mature or fully differentiated cell substitutes for another type of mature cell. Metaplasia may occur in epithelial or connective tissue cells. Dysplasia is generally considered a precursor of cancer, and is found mainly in the epithelia. Dysplasia is the most disorderly form of non-neoplastic cell growth, and involves a loss in individual cell uniformity and in the architectural orientation of cells. Dysplasia characteristically occurs where there exists chronic irritation or inflammation, and is often found in the cervix, respiratory passages, oral cavity, and gall bladder.

Alternatively, or in addition to the presence of abnormal cell growth characterized as hyperplasia, metaplasia, or dysplasia, the presence of one or more characteristics of a transformed or malignant phenotype displayed either in vivo or in vitro within a cell sample derived from a patient, is indicative of the desirability of prophylactic/therapeutic administration of a Therapeutic that possesses the ability to modulate activity of An aforementioned protein. Characteristics of a transformed phenotype include, but are not limited to: (i) morphological changes; (ii) looser substratum attachment; (iii) loss of cell-to-cell contact inhibition; (iv) loss of anchorage dependence; (v) protease release; (vi) increased sugar transport; (vii) decreased serum requirement; (viii) expression of fetal antigens, (ix) disappearance of the 250 kdal cell-surface protein, and the like. See e.g., Richards, et al., 1986. MOLECULAR PATHOLOGY, W.B. Saunders Co., Philadelphia, Pa.

In a specific embodiment of the present invention, a patient that exhibits one or more of the following predisposing factors for malignancy is treated by administration of an effective amount of a Therapeutic: (i) a chromosomal translocation associated with a malignancy (e.g., the Philadelphia chromosome (bcr/abl) for chronic myelogenous leukemia and t(14;18) for follicular lymphoma, etc.); (ii) familial polyposis or Gardner's syndrome (possible forerunners of colon cancer); (iii) monoclonal gammopathy of undetermined significance (a possible precursor of multiple myeloma) and (iv) a first degree kinship with persons having a cancer or pre-cancerous disease showing a Mendelian (genetic) inheritance pattern (e.g., familial polyposis of the colon, Gardner's syndrome, hereditary exostosis, polyendocrine adenomatosis, Peutz-Jeghers syndrome, neurofibromatosis of Von Recklinghausen, medullary thyroid carcinoma with amyloid production and pheochromocytoma, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia and Bloom's syndrome).

In another embodiment, a Therapeutic of the present invention is administered to a human patient to prevent the progression to breast, colon, lung, pancreatic, or uterine cancer, or melanoma or sarcoma.

### Hyperproliferative and Dysproliferative Disorders

In one embodiment of the present invention, a Therapeutic is administered in the therapeutic or prophylactic treatment of hyperproliferative or benign dysproliferative disorders. The efficacy in treating or preventing hyperproliferative diseases or disorders of a Therapeutic of the present invention may be assayed by any method known within the art. Such assays include in vitro cell proliferation assays, in vitro or in vivo assays using animal models of hyperproliferative diseases or disorders, or the like. Potentially effective Therapeutics may, for example, promote cell proliferation in culture or cause growth or cell proliferation in animal models in comparison to controls.

Specific embodiments of the present invention are directed to the treatment or prevention of cirrhosis of the liver (a condition in which scarring has overtaken normal liver regeneration processes); treatment of keloid (hypertrophic scar) formation causing disfiguring of the skin in which the scarring process interferes with normal renewal; psoriasis (a common skin condition characterized by excessive proliferation of the skin and delay in proper cell fate determination); benign tumors; fibrocystic conditions and tissue hypertrophy (e.g., benign prostatic hypertrophy).

### Neurodegenerative Disorders

SECX protein have been implicated in the deregulation of cellular maturation and apoptosis, which are both characteristic of neurodegenerative disease. Accordingly, Therapeutics of the invention, particularly but not limited to those that modulate (or supply) activity of an aforementioned protein, may be effective in treating or preventing neurodegenerative disease. Therapeutics of the present invention that modulate the activity of an aforementioned protein involved in neurodegenerative disorders can be assayed by any method known in the art for efficacy in treating or preventing such neurodegenerative diseases and disorders. Such assays include in vitro assays for regulated cell maturation or inhibition of apoptosis or in vivo assays using animal models of neurodegenerative diseases or disorders, or any of the assays described below. Potentially effective Therapeutics, for example but not by way of limitation, promote regulated cell maturation and prevent cell apoptosis in culture, or reduce neurodegeneration in animal models in comparison to controls.

Once a neurodegenerative disease or disorder has been shown to be amenable to treatment by modulation activity, that neurodegenerative disease or disorder can be treated or prevented by administration of a Therapeutic that modulates activity. Such diseases include all degenerative disorders involved with aging, especially osteoarthritis and neurodegenerative disorders.

### Disorders Related to Organ Transplantation

SECX has been implicated in disorders related to organ transplantation, in particular but not limited to organ rejection. Therapeutics of the invention, particularly those that modulate (or supply) activity, may be effective in treating or preventing diseases or disorders related to organ transplantation. Therapeutics of the invention (particularly Therapeutics that modulate the levels or activity of an aforementioned protein) can be assayed by any method known in the art for efficacy in treating or preventing such diseases and disorders related to organ transplantation. Such assays include in vitro assays for using cell culture models as described below, or in vivo assays using animal models of diseases and disorders related to organ transplantation, see e.g., below. Potentially effective Therapeutics, for example but not by way of limitation, reduce immune rejection responses in animal models in comparison to controls.

Accordingly, once diseases and disorders related to organ transplantation are shown to be amenable to treatment by modulation of activity, such diseases or disorders can be treated or prevented by administration of a Therapeutic that modulates activity.

### Cardiovascular Disease

SECX has been implicated in cardiovascular disorders, including in atherosclerotic plaque formation. Diseases such as cardiovascular disease, including cerebral thrombosis or hemorrhage, ischemic heart or renal disease, peripheral vascular disease, or thrombosis of other major vessel, and other diseases, including diabetes mellitus, hypertension, hypothyroidism, cholesterol ester storage disease, systemic lupus erythematosus, homocysteinemia, and familial protein or lipid processing diseases, and the like, are either directly or indirectly associated with atherosclerosis. Accordingly, Therapeutics of the invention, particularly those that modulate (or supply) activity or formation may be effective in treating or preventing atherosclerosis-associated diseases or disorders. Therapeutics of the invention (particularly Therapeutics that modulate the levels or activity) can be assayed by any method known in the art, including those described below, for efficacy in treating or preventing such diseases and disorders.

A vast array of animal and cell culture models exist for processes involved in atherosclerosis. A limited and non-exclusive list of animal models includes knockout mice for premature atherosclerosis (Kurabayashi and Yazaki, 1996, Int. Angiol. 15: 187-194), transgenic mouse models of atherosclerosis (Kappel et al., 1994, FASEB J. 8: 583-592), antisense oligonucleotide treatment of animal models (Callow, 1995, Curr. Opin. Cardiol. 10: 569-576), transgenic rabbit models for atherosclerosis (Taylor, 1997, Ann. N.Y. Acad. Sci 811: 146-152), hypercholesterolemic animal models (Rosenfeld, 1996, Diabetes Res. Clin. Pract. 30 Suppl.: 1-11), hyperlipidemic mice (Paigen et al., 1994, Curr. Opin. Lipidol. 5: 258-264), and inhibition of lipoxygenase in animals (Sigal et al., 1994, Ann. N.Y. Acad. Sci. 714: 211-224). In addition, in vitro cell models include but are not limited to monocytes exposed to low density lipoprotein (Frostegard et al., 1996, Atherosclerosis 121: 93-103), cloned vascular smooth muscle cells (Suttles et al., 1995, Exp. Cell Res. 218: 331-338), endothelial cell-derived chemoattractant exposed T cells (Katz et al., 1994, J. Leukoc. Biol. 55: 567-573), cultured human aortic endothelial cells (Farber et al., 1992, Am. J. Physiol. 262: H1088-1085), and foam cell cultures (Libby et al., 1996, Curr Opin Lipidol 7: 330-335). Potentially effective Therapeutics, for example but not by way of limitation, reduce foam cell formation in cell culture models, or reduce atherosclerotic plaque formation in hypercholesterolemic mouse models of atherosclerosis in comparison to controls.

Accordingly, once an atherosclerosis-associated disease or disorder has been shown to be amenable to treatment by modulation of activity or formation, that disease or disorder can be treated or prevented by administration of a Therapeutic that modulates activity.

### Cytokine and Cell Proliferation/Differentiation Activity

A SECX protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK.

The activity of a protein of the invention may, among other means, be measured by the following methods: Assays for T-cell or thymocyte proliferation include without limitation those described in: CURRENT PROTOCOLS IN IMMUNOLOGY, Ed by Coligan et al., Greene Publishing Associates and Wiley-Interscience (Chapter 3 and Chapter 7); Takai et al., J Immunol 137:3494-3500, 1986; Bertagnoili et al., J Immunol 145:1706-1712, 1990; Bertagnolli et al., Cell Immunol 133:327-341, 1991; Bertagnolli, et al., J Immunol 149:3778-3783, 1992; Bowman et al., J Immunol 152:1756-1761, 1994.

Assays for cytokine production and/or proliferation of spleen cells, lymph node cells or thymocytes include, without limitation, those described by Kruisbeek and Shevach, In: CURRENT PROTOCOLS IN IMMUNOLOGY. Coligan et al., eds. Vol 1, pp. 3.12.1-14, John Wiley and Sons, Toronto 1994; and by Schreiber, In: CURRENT PROTOCOLS IN IMMUNOLOGY. Coligan eds. Vol 1 pp. 6.8.1-8, John Wiley and Sons, Toronto 1994.

Assays for proliferation and differentiation of hematopoietic and lymphopoietic cells include, without limitation, those described by Bottomly et al., In: CURRENT PROTOCOLS IN IMMUNOLOGY. Coligan et al., eds. Vol 1 pp. 6.3.1-6.3.12, John Wiley and Sons, Toronto 1991; deVries et al., J Exp Med 173:1205-1211, 1991; Moreau et al., Nature 336:690-692, 1988; Greenberger et al., Proc Natl Acad Sci U.S.A. 80:2931-2938,1983; Nordan, In: CURRENT PROTOCOLS IN IMMUNOLOGY. Coligan et al., eds. Vol 1 pp. 6.6.1-5, John Wiley and Sons, Toronto 1991; Smith et al., Proc Natl Acad Sci U.S.A. 83:1857-1861, 1986; Measurement of human Interleukin 11-Bennett, et al. In: CURRENT PROTOCOLS IN IMMUNOLOGY. Coligan et al., eds. Vol 1 pp. 6.15.1 John Wiley and Sons, Toronto 1991; Ciarletta, et al., In: CURRENT PROTOCOLS IN IMMUNOLOGY. Coligan et al., eds. Vol 1 pp. 6.13.1, John Wiley and Sons, Toronto 1991.

Assays for T-cell clone responses to antigens (which will identify, among others, proteins that affect APC-T cell interactions as well as direct T-cell effects by measuring proliferation and cytokine production) include, without limitation, those described In: CURRENT PROTOCOLS IN IMMUNOLOGY. Coligan et al., eds., Greene Publishing Associates and Wiley-Interscience (Chapter 3Chapter 6, Chapter 7); Weinberger et al., Proc Natl Acad Sci USA 77:6091-6095, 1980; Weinberger et al., Eur J Immun 11:405411, 1981; Takai et al., J Immunol 137:3494-3500, 1986; Takai et al., J Immunol 140:508-512, 1988.

### Immune Stimulating or Suppressing Activity

A SECX protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by vital (e.g., HIV) as well as bacterial or fungal infections, or may result from autoimmune disorders. More specifically, infectious diseases causes by vital, bacterial, fungal or other infection may be treatable using a protein of the present invention, including infections by HIV, hepatitis viruses, herpesviruses, mycobacteria, Leishmania species., malaria species. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitus, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or energy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

Down regulating or preventing one or more antigen functions (including without limitation B lymphocyte antigen functions (such as, for example, B7), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to energize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc Natl Acad Sci USA, 89:11102-11105 (1992). In addition, murine models of GVHD (see Paul ed., FUNDAMENTAL IMMUNOLOGY, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and auto-antibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of auto-antibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythematosis in MRL/Ipr/Ipr mice or NZB hybrid mice, murine autoimmune collagen arthritis, diabetes mellitus in NOD mice and BB rats, and murine experimental myasthenia gravis (see Paul ed., FUNDAMENTAL IMMUNOLOGY, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic vital diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-vital immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II a chain protein and an MHC class II β chain protein to thereby express MHC class I or MHC class H proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods: Suitable assays for thymocyte or splenocyte cytotoxicity include, without limitation, those described In: CURRENT PROTOCOLS IN IMMUNOLOGY. Coligan et al., eds. Greene Publishing Associates and Wiley-Interscience (Chapter 3, Chapter 7); Herrmann et al., Proc Natl Acad Sci USA 78:2488-2492,1981; Herrmann et al., J Immunol 128:1968-1974,1982; Handa et al., J Immunol 135:1564-1572, 1985; Takai et al., J Immunol 137:3494-3500, 1986; Takai et al., J Immunol 140:508-512,1988; Herrmann et al., Proc Natl Acad Sci USA 78:2488-2492, 1981; Herrmann et al., J Immunol 128:1968-1974, 1982; Handa et al., J Immunol 135:1564-1572, 1985; Takai et al., J Immunol 137:3494-3500,1986; Bowman et al., J Virology 61:1992-1998; Takai et al., J Immunol 140:508-512, 1988; Bertagnolli et al., Cell Immunol 133:327-341, 1991; Brown et al., J Immunol 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J Immunol 144:3028-3033, 1990; and Mond and Brunswick In: CURRENT PROTOCOLS IN IMMUNOLOGY. Coligan et al., (eds.) Vol 1 pp. 3.8.1-3.8.16, John Wiley and Sons, Toronto 1994.

Mixed lymphocyte reaction (MLR) assays (which will identify, among others, proteins that generate predominantly Th1 and CTL responses) include, without limitation, those described In: CURRENT PROTOCOLS IN IMMUNOLOGY. Coligan et al., eds. Greene Publishing Associates and Wiley-Interscience (Chapter 3, Chapter 7); Takai et al., J Immunol 137:3494-3500, 1986; Takai et al., J Immunol 140:508-512, 1988; Bertagnolli et al., J Immunol 149:3778-3783, 1992.

Dendritic cell-dependent assays (which will identify, among others, proteins expressed by dendritic cells that activate naive T-cells) include, without limitation, those described in: Guery et al., J Immunol 134:536-544, 1995; Inaba et al., J Exp Med 173:549-559,1991; Macatonia et al., J Immunol 154:5071-5079,1995; Porgador et al., J Exp Med 182:255-260, 1995; Nair et al., J Virol 67:40624069, 1993; Huang et al., Science 264:961-965, 1994; Macatonia et al., J Exp Med 169:1255-1264, 1989; Bhardwaj et al., J Clin Investig 94:797-807, 1994; and Inaba et al., J Exp Med 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Res 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., Internat J Oncol 1:639-648, 1992.

Assays for proteins that influence early steps of T-cell commitment and development include, without limitation, those described in: Antica et al., Blood 84:111-117, 1994; Fine et al., Cell Immunol 155: 111-122, 1994; Galy et al., Blood 85:2770-2778, 1995; Toki et al., Proc Nat Acad Sci USA 88:7548-7551, 1991.

### Hematopoiesis Regulating Activity

A SECX protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods. Suitable assays for proliferation and differentiation of various hematopoietic lines are cited above. In addition, assays for embryonic stem cell differentiation (which will identify, among others, proteins that influence embryonic differentiation hematopoiesis) include, without limitation, those described in: Johansson et al. Cellular Biology 15:141-151, 1995; Keller et al., Mol. Cell. Biol. 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, In: CULTURE OF HEMATOPOIETIC CELLS. Freshney, et al. (eds.) Vol pp. 265-268, Wiley-Liss, Inc., New York, N.Y 1994; Hirayama et al., Proc Natl Acad Sci USA 89:5907-5911, 1992; McNiece and Briddeli, In: CULTURE OF HEMATOPOIETIC CELLS. Freshney, et al. (eds.) Vol pp. 23-39, Wiley-Liss, Inc., New York, N.Y. 1994; Neben et al., Exp Hematol 22:353-359, 1994; Ploemacher, In: CULTURE OF HEMATOPOIETIC CELLS. Freshney, et al. eds. Vol pp. 1-21, Wiley-Liss, Inc., New York, N.Y. 1994; Spooncer et al., In: CULTURE OF HEMATOPOIETIC CELLS. Freshhey, et al., (eds.) Vol pp. 163-179, Wiley-Liss, Inc., New York, N.Y. 1994; Sutherland, In: CULTURE OF HEMATOPOIETIC CELLS. Freshney, et al., (eds.) Vol pp. 139-162, Wiley-Liss, Inc., New York, N.Y. 1994.

### Tissue Growth Activity

A SECX protein of the present invention also may have utility in compositions used for bone, cartilage, tendon, ligament and/or nerve tissue growth or regeneration, as well as for wound healing and tissue repair and replacement, and in the treatment of bums, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendonitis, carpal tunnel syndrome and other tendon or ligament defects. The compositions may also include an appropriate matrix and/or sequestering agent as a career as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage. A protein of the present invention may also be useful for promoting or inhibiting differentiation of tissues described above from precursor tissues or cells; or for inhibiting the growth of tissues described above.

The activity of a protein of the invention may, among other means, be measured by the following methods. Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium). Assays for wound healing activity include, without limitation, those described in: Winter, EPIDERMAL WOUND HEALING, pp. 71-112 (Maibach and Rovee, eds.), Year Book Medical Publishers, Inc., Chicago, as modified by Eaglstein and Menz, J. Invest. Dermatol 71:382-84 (1978).

### Activin/Inhibin Activity

A SECX protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-b group, may be useful as a fertility inducing therapeutic, based upon the ability of activin molecules in stimulating FSH release from cells of the anterior pituitary. See, for example, U.S. Pat. No.4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc Natl Acad Sci USA 83:3091-3095, 1986.

### Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by following methods. Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: CURRENT PROTOCOLS IN IMMUNOLOGY, Coligan et al., eds. (Chapter 6.12, MEASUREMENT OF ALPHA AND BETA CHEMOKINES 6.12.1-6.12.28); Taub et al. J Clin Invest 95:1370-1376, 1995; Lind et al. APMIS 103:140-146, 1995; Muller et al., Eur J Immunol 25: 1744-1748; Gruber et al. J Immunol 152:5860-5867, 1994; Johnston et al., J Immunol 153: 1762-1768, 1994.

### Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26:131-140,1986; Burdick et al., Thrombosis Res. 45:413419, 1987; Humphrey et al., Fibrinolysis 5:71-79 (1991); Schaub, Prostaglandins 35:467-474, 1988.

### Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selectins, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: CURRENT PROTOCOLS IN IMMUNOLOGY, Ed by Coligan, et al., Greene Publishing Associates and Wiley-Interscience (Chapter 7.28, Measurement of Cellular Adhesion under static conditions 7.28.1-7.28.22), Takai et al., Proc Natl Acad Sci USA 84:6864-6868,1987; Bierer et al., J. Exp. Med. 168:1145-1156, 1988; Rosenstein et al., J. Exp. Med. 169:149-160 1989; Stoltenborg et al., J Immunol Methods 175:59-68, 1994; Stitt et al., Cell 80:661-670, 1995.

### Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

### Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

### Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

### Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining SECX protein and/or nucleic acid expression as well as SECX activity, in the context of a biological sample (e.g., blood, serum, cells, tissue) to thereby determine whether an individual is afflicted with a disease or disorder, or is at risk of developing a disorder, associated with aberrant SECX expression or activity. The invention also provides for prognostic (or predictive) assays for determining whether an individual is at risk of developing a disorder associated with SECX protein, nucleic acid expression or activity. For example, mutations in a SECX gene can be assayed in a biological sample. Such assays can be used for prognostic or predictive purpose to thereby prophylactically treat an individual prior to the onset of a disorder characterized by or associated with SECX protein, nucleic acid expression or activity.

Another aspect of the invention provides methods for determining SECX protein, nucleic acid expression or SECX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of SECX in clinical trials.

These and other agents are described in further detail in the following sections.

### Diagnostic Assays

An exemplary method for detecting the presence or absence of SECX in a biological sample involves obtaining a biological sample from a test subject and contacting the biological sample with a compound or an agent capable of detecting SECX protein or nucleic acid (e.g., mRNA, genomic DNA) that encodes SECX protein such that the presence of SECX is detected in the biological sample. Techniques that detect the presence of a SECX nucleic acid or protein can be modified by methods known in the art in order to quantitate relative or absolute concentrations of the SECX nucleic acid or protein being detected. Methods include, e.g., Northern or Southern blot hybridization (above) or quantitative PCR (below) for measuring SECX nucleic acid levels, and antibody detection (below), such as ELISA assays and antibody pull-down assay for measuring SECX polypeptide levels.

An agent for detecting SECX mRNA or genomic DNA is a labeled nucleic acid probe capable of hybridizing to SECX mRNA or genomic DNA. The nucleic acid probe can be, for example, a full-length SECX nucleic acid, or a portion thereof, such as an oligonucleotide of at least 15, 30, 50, 100, 250 or 500 nucleotides in length and sufficient to specifically hybridize under stringent conditions to SECX mRNA or genomic DNA. Presence or absence of a hybridization signal using a given probe with reveal the presence or absence of the nucleic acid complementary to the probe. Hybridization signal can be quantitated to measure the absolute or relative quantities of the nucleic acid being probed. Other suitable probes for use in the diagnostic assays of the invention are described herein.

An agent for detecting SECX protein is an antibody capable of binding to SECX protein, preferably an antibody with a detectable label. Antibodies can be polyclonal, or more preferably, monoclonal. An intact antibody, or a fragment thereof (e.g., F_{ab} or F_{(ab′)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect SECX mRNA, protein, or genomic DNA in a biological sample in vitro as well as in vivo. For example, in vitro techniques for detection of SECX mRNA include Northern hybridizations and in situ hybridizations. In vitro techniques for detection of SECX protein include enzyme linked immunosorbent assays (ELISAs), Western blots, immunoprecipitations and immunofluorescence. In vitro techniques for detection of SECX genomic DNA include Southern hybridizations. Furthermore, in vivo techniques for detection of SECX protein include introducing into a subject a labeled anti-SECX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting SECX protein, mRNA, or genomic DNA, such that the presence of SECX protein, mRNA or genomic DNA is detected in the biological sample, and comparing the presence of SECX protein, mRNA or genomic DNA in the control sample with the presence of SECX protein, mRNA or genomic DNA in the test sample.

The invention also encompasses kits for detecting the presence of SECX in a biological sample. For example, the kit can comprise: a labeled compound or agent capable of detecting SECX protein or mRNA in a biological sample; means for determining the amount of SECX in the sample; and means for comparing the amount of SECX in the sample with a standard. The compound or agent can be packaged in a suitable container. The kit can further comprise instructions for using the kit to detect SECX protein or nucleic acid.

### Prognostic Assays

The diagnostic methods described herein can furthermore be utilized to identify subjects having or at risk of developing a disease or disorder associated with aberrant SECX expression or activity. For example, the assays described herein, such as the preceding diagnostic assays or the following assays, can be utilized to identify a subject having or at risk of developing a disorder associated with SECX protein, nucleic acid expression or activity such as cancer or fibrotic disorders, or a SECX-specific disease as described in the individual sections 1-14, above. Alternatively, the prognostic assays can be utilized to identify a subject having or at risk for developing a disease or disorder. Thus, the present invention provides a method for identifying a disease or disorder associated with aberrant SECX expression or activity in which a test sample is obtained from a subject and SECX protein or nucleic acid (e.g., mRNA, genomic DNA) is detected, wherein the presence of SECX protein or nucleic acid is diagnostic for a subject having or at risk of developing a disease or disorder associated with aberrant SECX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant SECX expression or activity. For example, such methods can be used to determine whether a subject can be effectively treated with an agent for a disorder, such as cancer or preclampsia or a SECX-specific disease as described in the individual sections 1-14, above. Thus, the present invention provides methods for determining whether a subject can be effectively treated with an agent for a disorder associated with aberrant SECX expression or activity in which a test sample is obtained and SECX protein or nucleic acid is detected (e.g., wherein the presence of SECX protein or nucleic acid is diagnostic for a subject that can be administered the agent to treat a disorder associated with aberrant SECX expression or activity.)

The methods of the invention can also be used to detect genetic lesions in a SECX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a SECX-protein, or the mis-expression of the SECX gene. For example, such genetic lesions can be detected by ascertaining the existence of at least one of (1) a deletion of one or more nucleotides from a SECX gene; (2) an addition of one or more nucleotides to a SECX gene; (3) a substitution of one or more nucleotides of a SECX gene, (4) a chromosomal rearrangement of a SECX gene; (5) an alteration in the level of a messenger RNA transcript of a SECX gene, (6) aberrant modification of a SECX gene, such as of the methylation pattern of the genomic DNA, (7) the presence of a non-wild type splicing pattern of a messenger RNA transcript of a SECX gene, (8) a non-wild type level of a SECX-protein, (9) allelic loss of a SECX gene, and (10) inappropriate post-translational modification of a SECX-protein. As described herein, there are a large number of assay techniques known in the art which can be used for detecting lesions in a SECX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the SECX-gene (see Abravaya et al. (1995) Nucl Acids Res 23:675-682). This method can include the steps of collecting a sample of cells from a patient, isolating nucleic acid (e.g., genomic, mRNA or both) from the cells of the sample, contacting the nucleic acid sample with one or more primers that specifically hybridize to a SECX gene under conditions such that hybridization and amplification of the SECX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci USA 87:1874-1878), transcriptional amplification system (Kwoh, et al., 1989, Proc Natl Acad Sci USA 86:1173-1177), Q-Beta Replicase (Lizardi et al, 1988, BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a SECX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No.5,493,531) can be used to score for the presence of specific mutations by development or loss of a ribozyme cleavage site.

In other embodiments, genetic mutations in SECX can be identified by hybridizing a sample and control nucleic acids, e.g., DNA or RNA, to high density arrays containing hundreds or thousands of oligonucleotides probes (Cronin et al. (1996) Human Mutation 7: 244-255; Kozal et al. (1996) Nature Medicine 2: 753-759). For example, genetic mutations in SECX can be identified in two dimensional arrays containing light-generated DNA probes as described in Cronin et al. above. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the SECX gene and detect mutations by comparing the sequence of the sample SECX with the corresponding wild-type (control) sequence. Examples of sequencing reactions include those based on techniques developed by Maxim and Gilbert (1977) PNAS 74:560 or Sanger (1977) PNAS 74:5463. It is also contemplated that any of a variety of automated sequencing procedures can be utilized when performing the diagnostic assays (Naeve et al., (1995) BioTechniques 19:448), including sequencing by mass spectrometry (see, e.g., PCT International Publ. No. WO 94/16101; Cohen et al. (1996) Adv Chromatogr 36:127-162; and Griffin et al. (1993) Appl Biochem Biotechnol 38:147-159).

Other methods for detecting mutations in the SECX gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type SECX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc Natl Acad Sci USA 85:4397; Saleeba et al (1992) Methods Enzymol 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in SECX cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a SECX sequence, e.g., a wild-type SECX sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in SECX genes. For example, single strand conformation polymorphism (SSCP) may be used to detect differences in electrophoretic mobility between mutant and wild type nucleic acids (Orita et al. (1989) Proc Natl Acad Sci USA: 86:2766, see also Cotton (1993) Mutat Res 285:125-144; Hayashi (1992) Genet Anal Tech Appl 9:73-79). Single-stranded DNA fragments of sample and control SECX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol Cell Probes 6:1). It is anticipated that in certain embodiments amplification may also be performed using Taq ligase for amplification (Barany (1991) Proc Natl Acad Sci USA 88:189). In such cases, ligation will occur only if there is a perfect match at the 3′ end of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a SECX gene.

Furthermore, any cell type or tissue, preferably peripheral blood leukocytes, in which SECX is expressed may be utilized in the prognostic assays described herein. However, any biological sample containing nucleated cells may be used, including, for example, buccal mucosal cells.

### Pharmacogenomics

Agents, or modulators that have a stimulatory or inhibitory effect on SECX activity (e.g., SECX gene expression), as identified by a screening assay described herein can be administered to individuals to treat (prophylactically or therapeutically) disorders (e.g., cancer or gestational disorders or a SECX-specific disease as described in the individual sections 1-14, above) associated with aberrant SECX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of SECX protein, expression of SECX nucleic acid, or mutation content of SECX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, Clin Exp Pharmacol Physiol, 1996, 23:983-985 and Linder, Clin Chem, 1997, 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of SECX protein, expression of SECX nucleic acid, or mutation content of SECX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a SECX modulator, such as a modulator identified by one of the exemplary screening assays described herein.

### Monitoring of Effects During Clinical Trials

Monitoring the influence of agents (e.g., drugs, compounds) on the expression or activity of SECX (e.g., the ability to modulate aberrant cell proliferation and/or differentiation) can be applied not only in basic drug screening, but also in clinical trials. For example, the effectiveness of an agent determined by a screening assay as described herein to increase SECX gene expression, protein levels, or upregulate SECX activity, can be monitored in clinical trails of subjects exhibiting decreased SECX gene expression, protein levels, or downregulated SECX activity. Alternatively, the effectiveness of an agent determined by a screening assay to decrease SECX gene expression, protein levels, or downregulate SECX activity, can be monitored in clinical trails of subjects exhibiting increased SECX gene expression, protein levels, or upregulated SECX activity. In such clinical trials, the expression or activity of SECX and, preferably, other genes that have been implicated in, for example, a cellular proliferation disorder or a SECX-specific disease as described in the individual sections 1-14, above, can be used as a “read out” or markers of the immune responsiveness of a particular cell.

For example, and not by way of limitation, genes, including SECX, that are modulated in cells by treatment with an agent (e.g., compound, drug or small molecule) that modulates SECX activity (e.g., identified in a screening assay as described herein) can be identified. Thus, to study the effect of agents on cellular proliferation disorders, for example, in a clinical trial, cells can be isolated and RNA prepared and analyzed for the levels of expression of SECX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of SECX or other genes. In this way, the gene expression pattern can serve as a marker, indicative of the physiological response of the cells to the agent. Accordingly, this response state may be determined before, and at various points during, treatment of the individual with the agent.

In one embodiment, the present invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a SECX protein, mRNA, or genomic DNA in the preadministration sample; (iii) obtaining one or more post-administration samples from the subject; (iv) detecting the level of expression or activity of the SECX protein, mRNA, or genomic DNA in the post-administration samples; (v) comparing the level of expression or activity of the SECX protein, mRNA, or genomic DNA in the pre-administration sample with the SECX protein, mRNA, or genomic DNA in the post administration sample or samples; and (vi) altering the administration of the agent to the subject accordingly. For example, increased administration of the agent may be desirable to increase the expression or activity of SECX to higher levels than detected, i.e., to increase the effectiveness of the agent. Alternatively, decreased administration of the agent may be desirable to decrease expression or activity of SECX to lower levels than detected, i.e., to decrease the effectiveness of the agent.

### Methods of Treatment

The present invention provides for both prophylactic and therapeutic methods of treating a subject at risk of (or susceptible to) a disorder or having a disorder associated with aberrant SECX expression or activity.

### Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to, (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989, Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

### Prophylactic Methods

In one aspect, the invention provides a method for preventing, in a subject, a disease or condition associated with an aberrant SECX expression or activity, by administering to the subject an agent that modulates SECX expression or at least one SECX activity. Subjects at risk for a disease that is caused or contributed to by aberrant SECX expression or activity can be identified by, for example, any or a combination of diagnostic or prognostic assays as described herein. Administration of a prophylactic agent can occur prior to the manifestation of symptoms characteristic of the SECX aberrancy, such that a disease or disorder is prevented or, alternatively, delayed in its progression. Depending on the type of SECX aberrancy, for example, a SECX agonist or SECX antagonist agent can be used for treating the subject. The appropriate agent can be determined based on screening assays described herein. The prophylactic methods of the present invention are further discussed in the following subsections.

### Therapeutic Methods

Another aspect of the invention pertains to methods of modulating SECX expression or activity for therapeutic purposes. The modulatory method of the invention involves contacting a cell with an agent that modulates one or more of the activities of SECX protein activity associated with the cell. An agent that modulates SECX protein activity can be an agent as described herein, such as a nucleic acid or a protein, a naturally-occurring cognate ligand of a SECX protein, a peptide, a SECX peptidomimetic, or other small molecule. In one embodiment, the agent stimulates one or more SECX protein activity. Examples of such stimulatory agents include active SECX protein and a nucleic acid molecule encoding SECX that has been introduced into the cell. In another embodiment, the agent inhibits one or more SECX protein activity. Examples of such inhibitory agents include antisense SECX nucleic acid molecules and anti-SECX antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a SECX protein or nucleic acid molecule. In one embodiment, the method involves administering an agent (e.g., an agent identified by a screening assay described herein), or combination of agents that modulates (e.g., upregulates or downregulates) SECX expression or activity. In another embodiment, the method involves administering a SECX protein or nucleic acid molecule as therapy to compensate for reduced or aberrant SECX expression or activity.

Stimulation of SECX activity is desirable in situations in which SECX is abnormally downregulated and/or in which increased SECX activity is likely to have a beneficial effect. One example of such a situation is where a subject has a disorder characterized by aberrant cell proliferation and/or differentiation (e.g., cancer). Another example of such a situation is where the subject has a gestational disease (e.g., preclampsia). Other diseases of the invention include the SECX-specific diseases as described in the individual sections 1-14, above.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

This invention is further illustrated by the following examples, which should not be construed as limiting. The contents of all references, patents and published patent applications cited throughout this application are hereby incorporated by reference.

### EXAMPLES

### Example 1

### Preparation of Expression Vector pCEP4/Sec

The oligonucleotide primers, pSec-V5-His Forward 5′-CTCGTCCTCG AGGGTAAGCC TATCC CTAAC-3′ (SEQ ID NO:32) and pSec-V5-His Reverse 5′-CTCGTCGGGC CCCTGATCAG CGGGTTTAAA C-3′ (SEQ ID NO:33), were designed to amplify a fragment from the pcDNA3.1-V5His (Invitrogen, Carlsbad, Calif.) expression vector. The PCR product was digested with XhoI and ApaI and ligated into the XhoI/ApaI digested pSecTag2 B vector harboring an Ig kappa leader sequence (Invitrogen, Carlsbad Calif.). The correct structure of the resulting vector, pSecV5His, was verified by DNA sequence analysis. The vector pSecV5His was digested with PmeI and NheI, and the PmeI-NheI fragment was ligated into the BamHI/Klenow and NheI treated vector pCEP4 (Invitrogen, Carlsbad, Calif.). The resulting vector was named pCEP4/Sec.

### Example 2

### Molecular Cloning of 11753149

In this example, cloning is described for the Clone 11753149 cDNA from residue 32 to 326, which encodes the predicted mature protein without the membrane attachment domain predicted to start at residue 327.

Oligonucleotide primers were designed to PCR amplify the above 11753149 sequence. The forward primer includes an in-frame BglII restriction site (underlined): 11753149 SECF: 5′-CTCGTC AGATCT CGC AGC GGA GAT GCC ACC TTC CCC AAA G-3′ (SEQ ID NO:34), and the reverse primer contains an in-frame XhoI restriction site (underlined): 11753149 SECR: 5′-CTCGTC CTCGAG CCT CCT CGA CGT GCC GTT GCT CAC CTC G-3′ (SEQ ID NO:35).

Three separate PCR reactions were set up using 5 ng human fetal brain cDNA template, a total of 5 ng cDNA combined from equal amounts of cDNAs derived from human testis, human fetal brain, human mammary and human skeletal muscle, and 5 ng human testis cDNA template. The reaction mixtures contained 1 microM each of the 11753149 SECF and 11753149 SECR primers, 5 micromoles dNTP (Clontech Laboratories, Palo Alto Calif.) and 1 microliter of 50×Advantage-HF 2 polymerase (Clontech Laboratories, Palo Alto Calif.) in 50 microliter reaction volume. The following reaction conditions were used:
a) 96° C. 3 minutes
b) 96° C. 30 seconds denaturation
c) 60° C. 30 seconds, primer annealing.
d) 72° C. 2 minute extension.
Repeat steps (b)-(d) 35 times
e) 72° C. 5 minutes final extension

The expected 885 bp amplified product was detected by agarose gel electrophoresis in all samples. The fragment derived from human fetal brain was purified from agarose gel and ligated to pCR2.1 vector (Invitrogen, Carlsbad, Calif.). The cloned insert was sequenced and verified as an ORF encoding the predicted mature form of 11753149. The construct is called TA-1753149-S263D. The cloned sequence was determined to be 100% identical to the predicted one.

### Example 3

### Expression of h11753149 in Human Embryonic Kidney 293 Cells

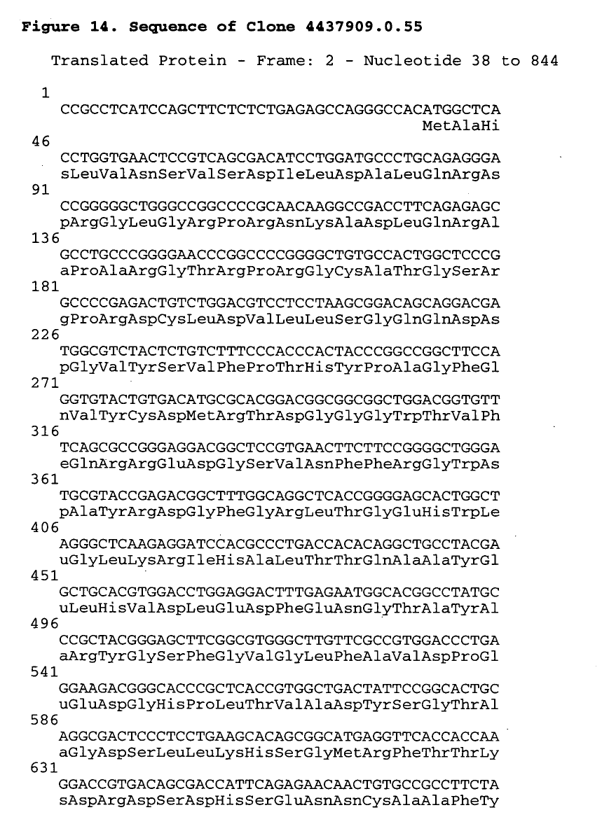

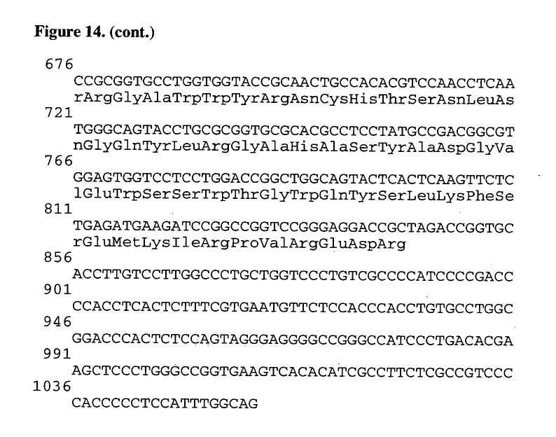

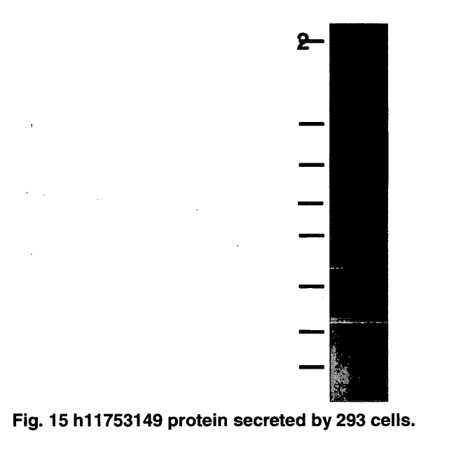

The BglII-XhoI fragment containing the human clone 11753149 sequence was isolated from 11753149-pCR2.1 and subcloned into the vector pCEP4/Sec to generate expression vector pCEP4/Sec-11753149. The pCEP4/Sec-11753149 vector was transfected into 293 cells using LipofectaminePlus™ reagent (Life Technologies, Rockville, Md.) following the manufacturer's instructions. The cell pellet and supernatant were harvested 72 hours after transfection and examined for h11753149 expression by Western blotting (reducing conditions) with an anti-V5 antibody. FIG. 15 shows that h11753149 is expressed as a protein secreted by 293 cells that is broadly distributed around 64 kDa, with a minor band at 35 kDa. The predicted molecular weight of the cloned fragment of 11753149 is about 32 kDa. The low intensity band, observed at about 35 kDa in FIG. 15, corresponds closely to the predicted molecular weight of the unmodified gene product. The program PROSITE predicts seven potential N-glycosylation sites in this fragment. Variable glycosylation of the recombinant 11753149 protein would account for the broad distribution of protein observed around 64 kDa in FIG. 15.

### Example 4

### Molecular Cloning of 3883556

This example describes cloning the cDNA coding for the predicted full length protein of clone 3883556. The following oligonucleotide primers were designed to PCR amplify the full length ORF. For downstream cloning purposes, the forward primer includes an in-frame BamHI restriction site and the reverse primer contains an in-frame XhoI restriction site. Restriction site sequences are underlined and the forward primer also includes an optimized Kozak sequence (in italics). The primer sequences used are 3883556 F-TOPO-F: GGA TCC ACC ATG AAT TTT CTG AAA TTA ATT GCT GTG TTT ATA G-3′ (SEQ ID NO:36), and 3883556 F-TOPO-R: 5′-CTC GAG ATT CAG CAG CTC CAG ACT CCC CCA TCC ATG-3′ (SEQ ID NO:37).

A PCR reaction was set up using 5 ng human fetal brain cDNA template. The reaction mixture contained 1 μM of each of the 3883556 F-TOPO-F and 3883556 F-TOPO-R primers, 5 micromoles dNTP (Clontech Laboratories, Palo Alto Calif.) and 1 μl of 50×Advantage-HF 2 polymerase (Clontech Laboratories, Palo Alto Calif.) in 50 μl reaction volume. The following reaction conditions were used:
a) 96° C. 3 minutes
b) 96° C. 30 seconds denaturation
c) 60° C. 30 seconds, primer annealing.
d) 72° C. 2 minute extension.
Repeat steps (b)-(d) 35 times
e) 72° C. 5 minutes final extension

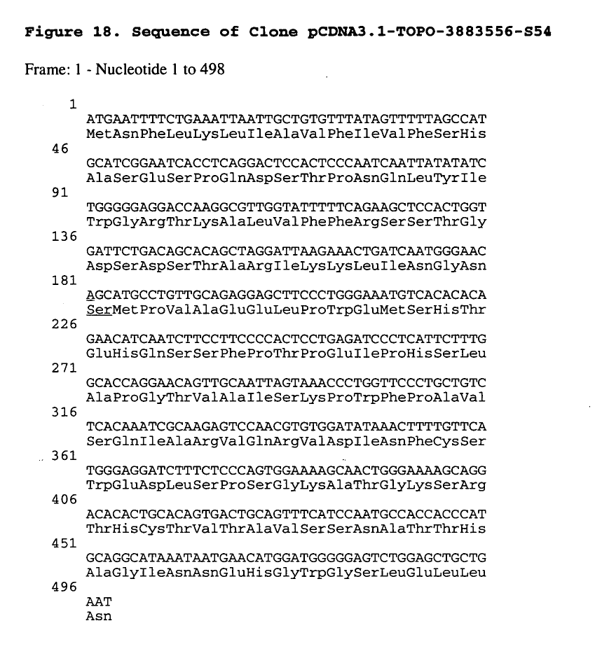

The expected 498 bp amplified product was detected by agarose gel electrophoresis. The fragment was purified from agarose gel and ligated to pCDNA3.1-TOPO-V5His vector (Invitrogen, Carlsbad, Calif.) following the manufacturer's recommendation. The cloned insert was sequenced and verified as an ORF coding for the predicted mature form of 3883556 with one basepair alteration. The construct is called pCDNA3.1-TOPO-3883556-S54 (SEQ ID NO:29), shown in FIG. 18. To verify the alteration, an independent PCR reaction was set up on a fetal brain template using identical conditions and parameters as above. The amplicon obtained in the second PCR was sequenced and the same alteration was again detected. The altered nucleotide yields an altered protein sequence (SEQ ID NO:30), shown in FIG. 18, compared to the original sequence of clone 3883556. The altered base and the altered amino acid residue are underlined in FIG. 18.

### Example 5

### Molecular Cloning of 4437909

This example describes the cloning of the sequence disclosed for clone 4437909. The segment coding for residues 31 to 269 of clone 4437909 was selected for cloning. The following oligonucleotide primers were designed to PCR amplify the cDNA. For downstream cloning purposes, the forward primer includes an in-frame BglII restriction site and the reverse primer contains an in-frame XhoI restriction site. Restriction site sequences are underlined. The primers used are 4437909-F: 5′-AGATC TCAGA GAGCG CCTGC CCGGG GAACC-3′ (SEQ ID NO:38), and 4437909-R: 5′-CTCGA GGCGG TCCTC CCGGA CCGGC CGGAT C-3′ (SEQ ID NO:39).

A PCR reaction was set up using a total of 5 ng cDNA, combined from equal amounts of human fetal brain, testis, mammary and skeletal muscle, as template. The reaction mixtures contained 1 microM of each of the 4437909 4437909-F and 4437909-R primers, 5 micromoles dNTP (Clontech Laboratories, Palo Alto Calif.) and 1 microliter of 50 ×Advantage-HF 2 polymerase (Clontech Laboratories, Palo Alto Calif.) in 50 microliter reaction volume. The following reaction conditions were used:
a) 96° C. 3 minutes
b) 96° C. 30 seconds denaturation
c) 60° C. 30 seconds, primer annealing.
d) 72° C. 2 minute extension.
Repeat steps (b)-(d) 35 times
e) 72° C. 5 minutes final extension

The expected 717 bp amplified product was detected by agarose gel electrophoresis. The fragment was purified from agarose gel and ligated to pCR2.1 vector (Invitrogen, Carlsbad, Calif.) following the manufacturer's recommendation. The cloned insert was sequenced using vector specific M13 Forward and M13 Reverse primers and the following gene-specific primers: 4437909 S1: 5′-GAGGACGGCTCCGTGAAC-3′ (SEQ ID NO:40), 4437909 S2: 5′-GTTCACGGAGCCGTCCTC-3′ (SEQ ID NO:41),4437909 S3: 5′-CAGCGGCATGAGGTTCACC-3′ (SEQ ID NO:42), and 4437909 S4: 5′-GGTGAACCTCATGCCGCTG-3′ (SEQ ID NO:43).

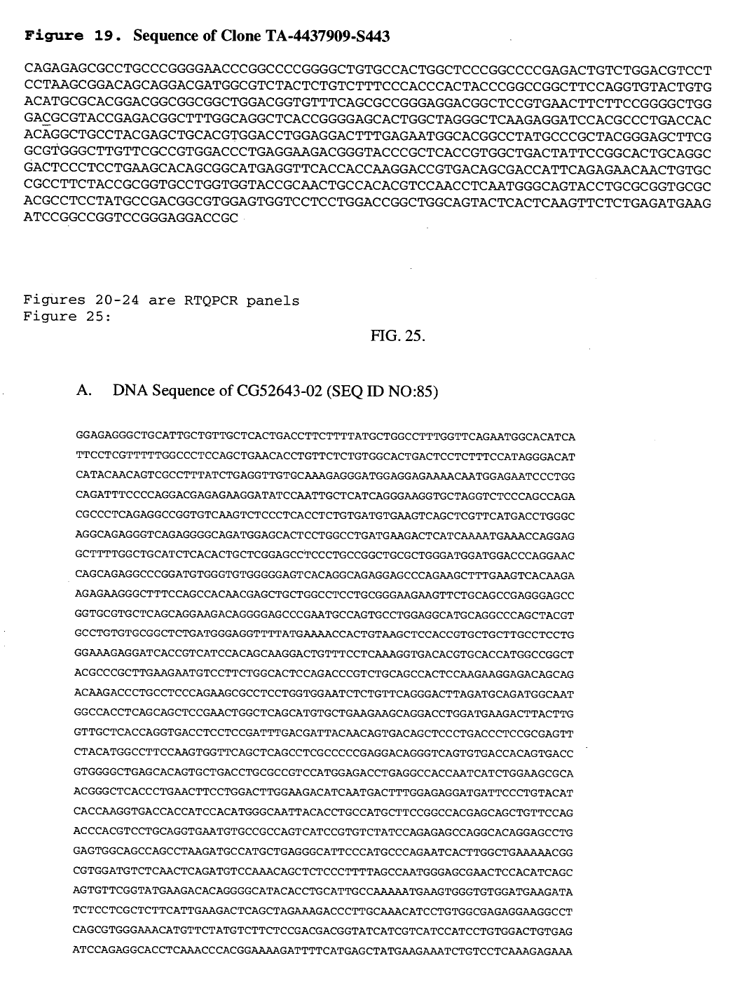

The construct is called TA-4437909-S443. The coding sequence in clone TA-4437909-S443 differs by one bp from that given for clone 4437909.0.4 (FIG. 13; SEQ ID NO:25). The sequence of clone TA-4437909-S443 (SEQ ID NO:31) is shown in FIG. 19, and the variant base is underlined. The base change, however, is silent and does not change the polypeptide sequence (SEQ ID NO:26) predicted for clone 4437909.0.4 (FIG. 13).

### Example 6

### Expression of h4437909 in Human Embryonic Kidney 293 Cells

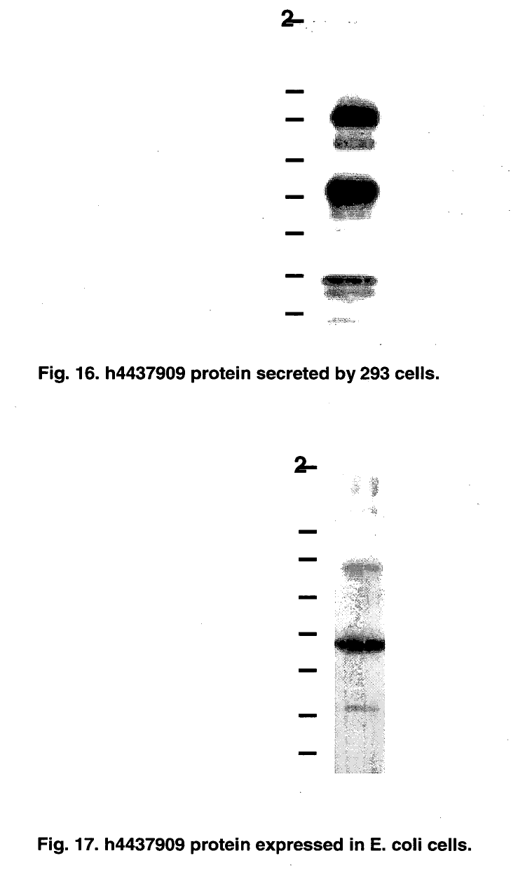

The BglII-XhoI fragment containing the human 4437909 sequence was isolated from plasmid 4437909-pCR2.1 and subcloned into the vector pCEP4/Sec to generate expression vector pCEP4/Sec-4437909. The pCEP4/Sec4437909 vector was transfected into 293 cells using the LipofectaminePlus™ reagent following the manufacturer's instructions (Gibco/BRL, Life Technologies, Inc., Rockville, Md.).). The cell pellet and supernatant were harvested 72 hours after transfection and examined for h4437909 expression by Western blotting (reducing conditions) with an anti-V5 antibody. FIG. 16 shows that h4437909 is expressed in 293 cells as three discrete secreted protein bands of 16, 40, and 70 kDa. The predicted molecular weight is 27064 Da. There is one N-glycosylation site predicted at residue 118. Accordingly it is believed that the band at about 40 kDa corresponds to the full sized monomeric form of the protein encoded by pCEP4/Sec-4437909.

### Example 7

### Expression of h4437909 in Recombinant E. coli Cells Using the Expression Vector pETMY-h4437909

The vector pRSETA (Invitrogen Inc., Carlsbad, Calif.) was digested with XhoI and NcoI restriction enzymes. Oligonucleotide linkers used include Linker 1: 5′-CATGGTCAGCCTAC-3′ (SEQ ID NO:44) and Linker 2: 5′-TCGAGTAGGCTGAC-3′ (SEQ ID NO:45). Linker 1 and Linker 2 were annealed at 37 degree Celsius and ligated into the XhoI-NcoI treated pRSETA. The resulting vector was confirmed by restriction analysis and sequencing and was named as pETMY. The BglIII-XhoI fragment (see above) was ligated into the pETMY that was digested with BamHI and XhoI restriction enzymes. The expression vector is named as pETMY-4437909. In this vector, h4437909 was fused to the 6×His tag and T7 epitope at its N-terminus. The plasmid pETMY-4437909 was then transformed into the E. coli expression host BL21 (DE3, pLys) (Novagen, Madison, Wis.). Expression of protein 4437909 was induced according to the manufacturer's instructions. After induction, total cells were harvested, and proteins were analyzed by Western blotting using anti-HisGly antibody (Invitrogen, Carlsbad, Calif.). FIG. 17 shows h4437909 was expressed as a 34 kDa protein in E. coli cells. This corresponds approximately to the molecular weight of 27064 Da expected for the protein encoded by pETMY-4437909.

### Example 8

### Quantitative Expression Analysis of 3883556 Nucleic Acid

The quantitative expression of clone 3883556 was assessed in 41 normal and 55 tumor samples (see Table 2) by real time quantitative PCR (TAQMAN®) performed on a Perkin-Elmer Biosystems ABI PRISM® 7700 Sequence Detection System.

First, 96 RNA samples were normalized to beta-actin and GAPDH. RNA (˜50 ng total RNA or ˜1 ng polyA+RNA) was converted to cDNA using the TAQMAN® Reverse Transcription Reagents Kit (PE Biosystems, Foster City, Calif.; cat # N808-0234) and random hexamers according to the manufacturer's protocol. Reactions were performed in 20 μl and incubated for 30 min. at 48° C. The cDNA (5 μl) was then transferred to a separate plate for the TAQMAN® reaction using beta-actin and GAPDH TAQMAN® Assay Reagents (PE Biosystems; cat. #'s 4310881E and 4310884E, respectively) and TAQMAN® universal PCR Master Mix (PE Biosystems; cat #4304447) according to the manufacturer's protocol. Reactions were performed in 25 μl using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between two samples being represented as 2 to the power of delta CT (i.e., 2^{ΔCT}). The average CT values obtained for β-actin and GAPDH were used to normalize RNA samples. The RNA sample generating the highest CT value required no further diluting, while all other samples were diluted relative to this sample according to their beta-actin /GAPDH average CT values.

Normalized RNA (5 μl) was converted to cDNA and analyzed via TAQMAN® using One Step RT-PCR Master Mix Reagents (PE Biosystems; cat. #4309169) and gene-specific primers according to the manufacturer's instructions. Probes and primers were designed for each assay according to Perkin Elmer Biosystem's Primer Express Software package (version I for Apple Computer's Macintosh Power PC) using the sequence of 3352358 as input. The primers used were Ag 45 (F): 5′-TCCCTGGGAA ATGTCACACA-3′ (SEQ ID NO:46) and Ag 45 (R): 5′-TTCCTGGTGC CAAAGAATGA G-3′ (SEQ ID NO:47), and the labeled probe was Ag 45 (P): TET-5′-AGAACATCAA TCTTCCTTCC CCACTCCTGA G-3′-TAM (SEQ ID NO:48).

Default settings were used for reaction conditions and the following parameters were set before selecting primers: primer concentration =250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size=75 bp to 100 bp. The probes and primers selected (see below) were synthesized by Synthegen (Houston, Tex., USA). Probes were double HPLC purified to remove uncoupled dye and evaluated by mass spectroscopy to verify coupling of reporter and quencher dyes to the 5′ and 3′ ends of the probe, respectively. The final concentrations for the forward and reverse primers were 900 nM each, and probe concentration was 200 nM.

PCR conditions: Normalized RNA from each tissue and each cell line was spotted in each well of a 96 well PCR plate (Perkin Elmer Biosystems). PCR cocktails including two probes (SEQX-specific and another gene-specific probe multiplexed with the SEQX probe) were set up using 1×TaqMan™ PCR Master Mix for the PE Biosystems 7700, with 5 mM MgCl₂, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by PCR amplification cycles as follows: 95° C. 10 min, then 40 cycles of 95° C for 15 seconds, 60° C. for 1 minute.

The TaqMan panel is shown in FIG. 20, Panels A, B and C. Tissues with high levels of expression include fetal brain, cerebellum, hippocampus, hypothalamus, liver, kidney, breast cancer, and uterus. These results suggest therapeutic indications for targeting 3883556 include brain tumors, preferably astrocytomas and gliomas; carcinomas such as selected renal cell and ovarian carcinomas.

TABLE 2
Tissue Sources of RNA

| | |
|---|---|
| 1 | Endothelial cells |
| 2 | Endothelial cells (treated) |
| 3 | Pancreas |
| 4 | Pancreatic ca. CAPAN 2 |
| 5 | Adipose |
| 6 | Adrenal gland |
| 7 | Thyroid |
| 8 | Salivary gland |
| 9 | Pituitary gland |
| 10 | Brain (fetal) |
| 11 | Brain (whole) |
| 12 | Brain (amygdala) |
| 13 | Brain (cerebellum) |
| 14 | Brain (hippocampus) |
| 15 | Brain (hypothalamus) |
| 16 | Brain (substantia nigra) |
| 17 | Brain (thalamus) |
| 18 | Spinal cord |
| 19 | CNS ca. (glio/astro) - U87-MG |
| 20 | CNS ca. (glio/astro) - U-118-MG |
| 21 | CNS ca. (astro) - SW1783 |
| 22 | CNS ca.* (neuro; met) SK-N-AS |
| 23 | CNS ca. (astro) - SF-539 |
| 24 | CNS ca. (astro) - SNB-75 |
| 25 | CNS ca. (glio) - SNB-19 |
| 26 | CNS ca. (glio) - U251 |
| 27 | CNS ca. (glio) - SF-295 |
| 28 | Heart |
| 29 | Skeletal muscle |
| 30 | Bone marrow |
| 31 | Thymus |
| 32 | Spleen |
| 33 | Lymph node |
| 34 | Colon (ascending) |
| 35 | Stomach |
| 36 | Small intestine |
| 37 | Colon ca./GI tract - SW480 |
| 38 | Colon ca.*/GI tract - (SW480 met)SW620 |
| 39 | Colon ca./GI tract - HT29 |
| 40 | Colon ca./GI tract - HCT-116 |
| 41 | Colon ca./GI tract - CaCo-2 |
| 42 | Colon ca./GI tract - HCT-15 |
| 43 | Colon ca./GI tract - HCC-2998 |
| 44 | Gastric ca.*/GI tract - (liver met) NCI-N87 |
| 45 | Bladder |
| 46 | Trachea |
| 47 | Kidney |
| 48 | Kidney (fetal) |
| 49 | Renal ca./Kidney - 786-0 |
| 50 | Renal ca./Kidney - A498 |
| 51 | Renal ca./Kidney - RXF 393 |
| 52 | Renal ca./Kidney - ACHN |
| 53 | Renal ca./Kidney - UO-31 |
| 54 | Renal ca./Kidney - TK-10 |
| 55 | Liver |
| 56 | Liver (fetal) |
| 57 | Liver ca. (hepatoblast) HepG2 |
| 58 | Lung |
| 59 | Lung (fetal) |
| 60 | Lung ca. (small cell) - LX-1 |
| 61 | Lung ca. (small cell) - NCI-H69 |
| 62 | Lung ca. (s. cell var.) - SHP-77 |
| 63 | Lung ca. (large cell) - NCI-H460 |
| 64 | Lung ca. (non-sm. cell) - A549 |
| 65 | Lung ca. (non-s. cell) - NCI-H23 |
| 66 | Lung ca (non-S. C.) - HOP-62 |
| 67 | Lung ca. (non-s. cl) - NCI-H522 |
| 68 | Lung ca. (squam.) - SW 900 |
| 69 | Lung ca. (squam.) - NCI-H596 |
| 70 | Mammary gland |
| 71 | Breast ca.* (pl. effusion) - MCF-7 |
| 72 | Breast ca.* (pl. ef) MDA-MB-231 |
| 73 | Breast ca.* (pl. effusion) - T47D |
| 74 | Breast ca. BT-549 |
| 75 | Breast ca. MDA-N |
| 76 | Ovary |
| 77 | Ovarian ca. - OVCAR-3 |
| 78 | Ovarian ca. - OVCAR-4 |
| 79 | Ovarian ca. - OVCAR-5 |
| 80 | Ovarian ca. - OVCAR-8 |
| 81 | Ovarian ca. - IGROV-1 |
| 82 | Ovarian ca.* - (ascites) SK-OV-3 |
| 83 | Myometrium |
| 84 | Uterus |
| 85 | Placenta |
| 86 | Prostate |
| 87 | Prostate ca.* (bone met)PC-3 |
| 88 | Testis |
| 89 | Melanoma/Skin - Hs688(A).T |
| 90 | Melanoma*/Skin - (met) Hs688(B).T |
| 91 | Melanoma/Skin - UACC-62 |
| 92 | Melanoma/Skin - M14 |
| 93 | Melanoma/Skin - LOX IMVI |
| 94 | Melanoma*/Skin - (met) SK-MEL-5 |
| 95 | Melanoma/Skin - SK-MEL-28 |
| 96 | Melanoma/Skin - UACC-257 |

### Example 9

### Quantitative Expression Analysis of 4324229 Nucleic Acids

The quantitative expression of clone 4324229 was assessed in normal and tumor by real time quantitive PCR (TAQMAN®) as described in EXAMPLE 8. The 4324229 clone-specific primers used were Ab10(F): 5′-GCCTGGCTCTCTGGATAGACA-3′ (SEQ ID NO:49) and Ab10(R): 5′-CACGAGCAGC TGTTCCAGAC-3′ (SEQ ID NO:50), and the labeled probe was Ab10(P):-FAM-5′-TGGCGGCACA TTCACCTGCA G-3′-TAMRA (SEQ ID NO:51). The tissue sources are listed in Table 2. The results are shown in FIG. 21, Panels A, B and C. Among the tissues showing a high level expression is lung (NCI-H460). The results suggest that therapeutic indications for targeting 4324229 include selected lung, breast and ovarian carcinomas.

### Example 10

### Quantitative Expression Analysis of 4339264 Nucleic Acid

The quantitative expression of clone 4339264 was assessed in normal and tumor by real time quantitive PCR (TAQMAN™) as described in Example 8. The clone-specific primers used were Ag 120 (F): 5′-AAAGGCGGAGGAAAGAAGTACTC-3′ (SEQ ID NO:52) and Ag 120 (R): 5′-GCTCCCGTTC CCTCTCCA-3′ (SEQ ID NO:53), and the labeled probe was Ag 120 (P): FAM-5′-CCTCTTTGTT CTTCTTGCCC GAGTTTTCTT T-3′-TAMRA (SEQ ID NO:54).

The tissue sources are listed in Table 2. The results are shown in FIG. 22, Panels A, B and C. The highest expression was shown in Prostate ca (bone met) PC-3. Other tissues showing a high level expression include adipose, colon ca. (HCT-116), renal ca. ( A498), lung ca. (S. CELL var.) SHP-77, lung ca. (large cell) NCI-H460, ovary, testis and melanoma (LOX IMVI). Furthermore the results suggest that clone 4339264 is downregulated in astrocytoma, when compared with normal brain or glioma. Therefore restoring the expression of 4339264 could be useful for the treatment of astrocytomas. Since it is a multiple membrane spanning protein, some kind of gene therapy would be necessary.

### Example 11

### Quantitative Expression Analysis of 4391184 Nucleic Acid

The quantitative expression of clone 4391184 was assessed in normal and tumor by real time quantitative PCR (TAQMAN®) as described in Example 8. The clone-specific primers used were Ab11(F): 5′-TGGAAGTCCCTCGGTAAAGGA-3′ (SEQ ID NO:55) and Ab11 (R): 5′-AGGACACCTG TGCCCTGTCT-3′ (SEQ ID NO:56), and the labeled probe was Ab11 (P): FAM-5′-CCCGCCTTGC CATTCCCTTC A -3′-TAMRA (SEQ ID NO:57).

The tissue sources are listed in Table 2. The results are shown in FIG. 23, Panels (A), (B) and (C). The highest expression was shown in prostate ca. (bone met) PC-3. Other tissues showing a high level expression include adipose, colon ca. (HCT-116), renal ca. ( A498), lung ca. (S. CELL var.) SHP-77, lung ca. (large cell) NCI-H460, ovary, testis and melanoma (LOX IMVI). Furthermore the results suggest that clone 4391184 is downregulated in astrocytoma, when compared with normal brain or glioma. Therefore restoring the expression of 4339264 could be useful for the treatment of astrocytomas. Since it is a multiple membrane spanning protein, some kind of gene therapy would be necessary.

Clone 4391184 is up-regulated in hematopoietic organs, bone marrow, thymus, spleen and lymph node, therefore it can have a hematopoietic activity, useful as protein drug after chemotherapy treatment. This exclude a systemic delivery of a targeting agent. If specific delivery could be achieved, targeting agent could be useful to treat breast tumors and selected melanomas.

### Example 12

### Radiation Hybrid Mapping Identifies the Chromosomal Location of Clones of the Invention

Radiation hybrid mapping using human chromosome markers was carried out for many of the clones described in the present invention. The procedure used to obtain these results is analogous to that described in Steen et al. (A High-Density Integrated Genetic Linkage and Radiation Hybrid Map of the Laboratory Rat, Genome Research 1999 (Published Online on May 21, 1999) Vol. 9, AP1-AP8, 1999). A panel of 93 cell clones containing the randomized radiation-induced human chromosomal fragments was screened in 96 well plates using PCR primers designed to identify the sought clones in a unique fashion. The results are presented in Table 3, which provides four columns giving, respectively, the clone number, the chromosome on which the clone is found, the distance in cR from a marker gene to the sought clone, and the identity of the marker gene.

TABLE 3
Radiation Hybrid Mapping Results for Clones of the Invention

| | Chromosome | Distance, | |
|---|---|---|---|
| Clone No. | No. | cR | Marker Gene |
| 4339264 | 19 | 6.40 | WI-5264 |
| | | 311.50 | IB1264 |
| 4437909 | 9 | 1.41 | WI-6494 |
| | | 426.40 | CHLC.GCT3G05 |

### Example 13

### Quantitative Expression Analysis of 4324229 Nucleic Acids

The quantitative expression of clone 4324229-02 was assessed in normal and tumor by real time quantitative PCR (TAQMAN®) as described in EXAMPLE 8. The 4324229-02 clone-specific primers used were as follows:

| | | | | |
|---|---|---|---|---|
| | | | | SEQ ID |
| Primers | Sequences | Tm | Start | NO: |

| | | | | |
|---|---|---|---|---|
| Ag22822 | 5′-TCTTCATCCAGGTCCTGCTT-3′ | 59.8 | 850 | 59 |
| Forward | | | | |
| Ag22822 | TET-5′-CTTCAGCACATGCTGAGCCAGTTCG- | 71.2 | 870 | 60 |
| Probe | 3′-TAMRA | | | |
| Ag22822 | 5′-TTCAGGGACTTAGATGCAGATG-3′ | 59.3 | 917 | 61 |
| Reverse | | | | |
| Ag 2812 | 5′-CTGTACTCGCTTTGTGGTTCA-3′ | 59 | 2968 | 62 |
| Forward | | | | |
| Ag 2812 | TET-5′-CACTGGTCTCCTTGCAAGTTTCCTAG- | 65.5 | 2990 | 63 |
| Probe | 3′-TAMRA | | | |
| Ag 2812 | 5′-AATCTTGGTAGCAGCGCATAC-3′ | 59.4 | 3022 | 64 |
| Reverse | | | | |

The tissue sources are listed in Table 2. The results are shown in FIG. 24. Among the tissues showing a high level of expression is, for example but not limited to lung (NCI-H460). The results suggest that therapeutic indications for targeting 4324229 include selected lung, breast, bladder and ovarian carcinomas. For example, therapeutic targeting with an antibody or domain thereof will block the migration and growth of cancer cells and promote cell death in cells where the gene is overexpressed in the tumor compared to normal adjacent tissues.

### Example 14

### Molecular Cloning of CG52676-02

The cDNA coding for the mature form of CG52676-02 (also known as 4357764-3) from residue 22 to 142 was targeted for “in-frame” cloning by PCR. The PCR template is based on human cDNA(s).

The following oligonucleotide primers were used to clone the target cDNA sequence:

| | | SEQ |
|---|---|---|
| Prim- | | ID |
| ers | Sequences | NO: |
| F1 | 5′-AAGCTTTCAGAAGTGGAATACAGAGCGGAGGTCG-3′ | 65 |
| R1 | 5′-CTCGAGTTCATAAAGATGGCATGCAAATGTCCACTC- | 66 |
| | 3′ | |

For downstream cloning purposes, the forward primer includes an in-frame HindIII restriction site and the reverse primer contains an in-frame XhoI restriction site.

Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus. PCR was performed using the above primers and 0.5ng-1.0 ng of one of the following human tissue cDNAs: skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM dNTP (Clontech Laboratories, Palo Alto Calif.) and 1 microliter of 50×Advantage-HF 2 polymerase (Clontech Laboratories) in 50 microliter-reaction volume. The following reaction conditions were used:

PCR condition 1:
a) 96° C. 3 minutes
b) 96° C. 30 seconds denaturation
c) 60° C. 30 seconds, primer annealing
d) 72° C. 6 minutes extension
Repeat steps b-d 15 times
e) 96° C. 15 seconds denaturation
f) 60° C. 30 seconds, primer annealing
g) 72° C. 6 minutes extension
Repeat steps e-g 29 times
e) 72° C. 10 minutes final extension

PCR condition 2:
a) 96° C. 3 minutes
b) 96° C. 15 seconds denaturation
c) 76° C. 30 seconds, primer annealing, reducing the temperature by 1° C. per cycle
d) 72° C. 4 minutes extension
Repeat steps b-d 34 times
e) 72° C. 10 minutes final extension

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers. The insert assembly 191998702 was found to encode an open reading frame between residues 22 and 142 of the CG52676-02 sequence. The polynucleotide sequence of 191998702 is given in FIG. 11B as SEQ ID NO:144 and the polypeptide sequence is given as (SEQ ID NO:145). FIG. 11C provides a ClustalW alignment of the polypeptide sequence of Clone 191998702 (SEQ ID NO:145) with the polypeptide sequence of CG52676-02 (SEQ ID NO: 146) and is about 100% identical to the sequence CG52676-02.

### Example 15

### Exon Linking of CG52703-03

Exon Linking: The cDNA coding for the nucleic acid CG52703-03 was cloned by the polymerase chain reaction (PCR) using the primers:

| | | SEQ ID |
|---|---|---|
| Primers | Sequences | NO: |
| F1 | 5′-GGGGGAATGAATAGCTCCCCTCACTT-3′ | 67 |
| R1 | 5′-ACTCACTATAGGGCTCGAGCGGCTG-3′ | 68 |

Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations. Physical clone: The PCR product derived by exon linking, covering the entire open reading frame, was cloned into the pCR2.1 vector from Invitrogen to provide clone 4002473_{—}1.699520.A7.

### Example 16

### Molecular Cloning of CG52643-02

Clone CG52643-02 encodes a follistatin-like protein, similar to clones AC012614_{—}1.0.123 and 4324229-2 described above, and has the same properties and uses described for these clones, i.e., regulation of cellular proliferation, cancers, and reproduction.

Exon Linking: The cDNA coding for the full-length of CG52643-02 from residue 1 to 842 and 23-842 were targeted for “in-frame” cloning by PCR. The PCR template is based on the previously identified plasmid, when available, or on human cDNA(s).

| | | |
|---|---|---|
| | | SEQ ID |
| Primers | Sequences | NO: |

| | | |
|---|---|---|
| 259341359 | 5′-CTCGAG ATGAAACCAGGAGGCTTTTGGCTGCATCTC-3′ | 69 |
| F1 | | |
| 259341359 | 5′-CTCGAG TACCTCACCCACCCACACCACTGTGGTCC-3′ | 70 |
| R1 | | |
| 268824728 | 5′-CTCGAG ATGAAACCAGGAGGCTTTTGGCTGCATCTC-3′ | 89 |
| F1 | | |
| 268824728 | 5′-CTCGAG TACCTCACCCACCCACACCACTGTGGTCC-3′ | 90 |
| R1 | | |
| 268825987 | 5′-CTCGAG ATGAAACCAGGAGGCTTTTGGCTGCATCTC-3′ | 91 |
| F1 | | |
| 268825987 | 5′-CTCGAG TACCTCACCCACCCACACCACTGTGGTCC-3′ | 92 |
| R1 | | |
| 268825997 | 5′-CTCGAG TGGATGGACCCAGGAACCAGCAGAGGCCC-3′ | 93 |
| F2 | | |
| 268825997 | 5′-CTCGAG TACCTCACCCACCCACACCACTGTGGTCC-3′ | 94 |
| R1 | | |
| 275698334 | 5′-CTCGAG | 129 |
| F1 | ATGAAACCAGGAGGCTTTTGGCTGCATCTC-3′ | |
| 275698334 | 5′-CTCGAG TACCTCACCCACCCACACCACTGT | 130 |
| R1 | GGTCC-3′ | |

For downstream cloning purposes, the forward primer includes an in-frame Xho I restriction site and the reverse primer contains an in-frame Xho I restriction site. Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus. When the tissue of expression is known and available, the second PCR was performed using the above primers and 0.5 ng-1.0 ng of one of the following human tissue cDNAs: skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus. The reaction mixtures contained 2 microliters of each of the primers (original concentration: 5 pmol/ul), 1 microliter of 10 mM dNTP (Clontech Laboratories, Palo Alto Calif.) and 1 microliter of Pfu DNA polymerase (Strategene) in 50 microliter-reaction volume. The following reaction conditions were used:

PCR condition 1:
a) 96° C. 3 minutes
b) 96° C. 30 seconds denaturation
c) 60° C. 30 seconds, primer annealing
d) 72° C. 6 minutes extension
Repeat steps b-d 15 times
e) 96° C. 15 seconds denaturation
f) 60° C. 30 seconds, primer annealing
g) 72° C. 6 minutes extension
Repeat steps e-g 29 times
e) 72° C. 10 minutes final extension

PCR condition 2:
a) 96° C. 3 minutes
b) 96° C. 15 seconds denaturation
c) 76° C. 30 seconds, reducing the temperature by 1° C. per cycle
d) 72° C. 4 minutes extension
Repeat steps b-d 34 times
e) 72° C. 10 minutes final extension.

An amplified product was detected by agarose gel electrophoresis. The fragment was gel-purified and ligated into the pCR2.1-TOPO vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers for assembly 259341359:

| | | SEQ ID |
|---|---|---|
| Primers | Sequences | NO: |
| SF1 | 5′-GAAAACCACTGTAAGCTCCACCG-3′ | 71 |
| SF2 | 5′-AGGTGACCTCCTCCGATTTGACGATTA-3′ | 72 |
| SF3 | 5′-TACATCACCAAGGTGACCACCATCC-3′ | 73 |
| SF4 | 5′-GAAGATATCTCCTCGCTCTTCATTG-3′ | 74 |
| SF5 | 5′-CAAGCCCAGAAAGTCCTACAGTCCA-3′ | 75 |
| SF6 | 5′-ACAATGATGCCCCTCAAGACCAT-3′ | 76 |
| SF7 | 5′-CAATCAATACAACATCTACGCGGCT-3′ | 77 |
| SR1 | 5′-TTTCATAAAACCTCCCATCAGAGCC-3′ | 78 |
| SR2 | 5′-CAAGTAAGTCTTCATCCAGGTCCTGCT-3′ | 79 |
| SR3 | 5′-GTGTAATTGCCCATGTGGATGGT-3′ | 80 |
| SR4 | 5′-TATCTTCATCCACACCCACTTCATT-3′ | 81 |
| SR5 | 5′-GATGTCGACCACAAGGACTCTGC-3′ | 82 |
| SR6 | 5′-GGTCTTGAGGGGCATCATTGTTT-3′ | 83 |
| SR7 | 5′-TGATTGCTTTCAGTGAAGGAGCG-3′ | 84 |

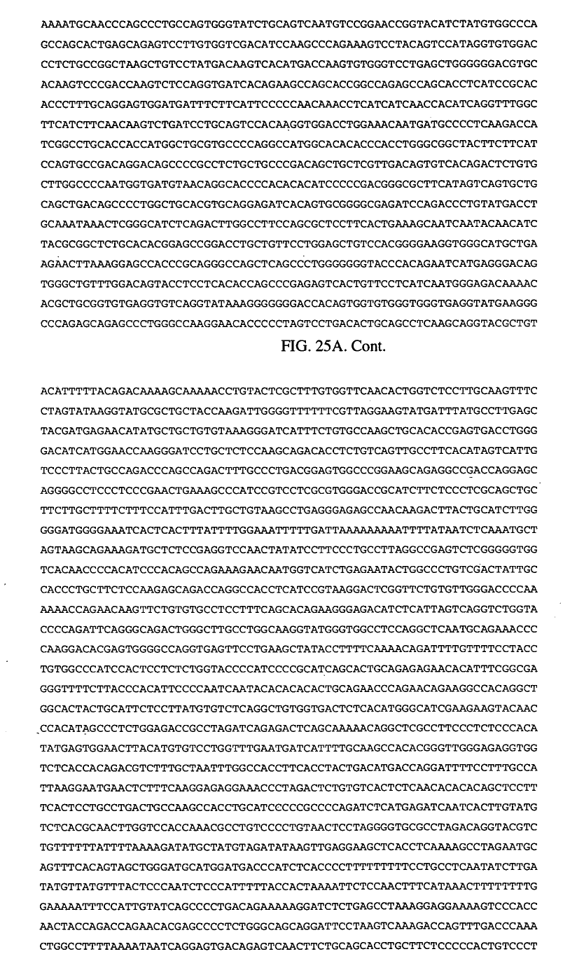

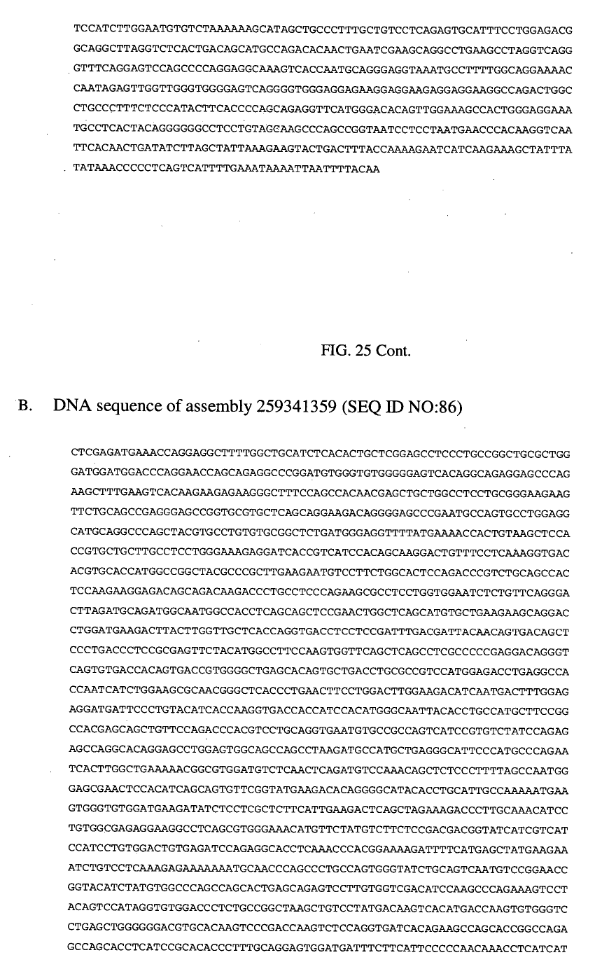

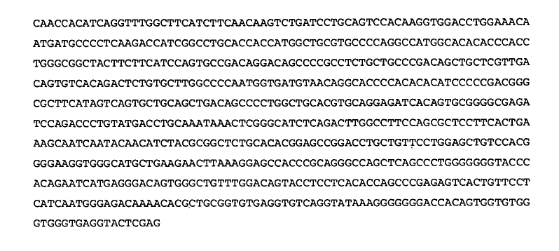

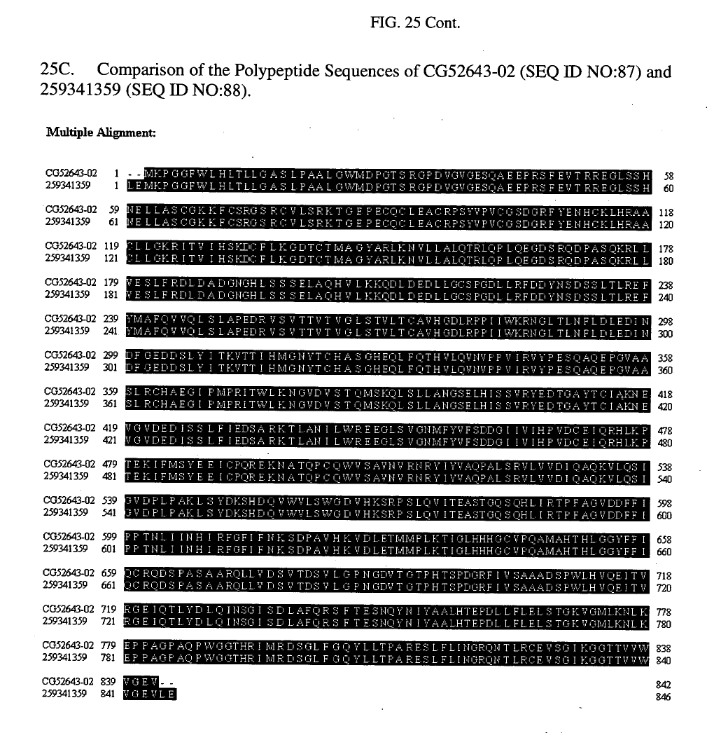

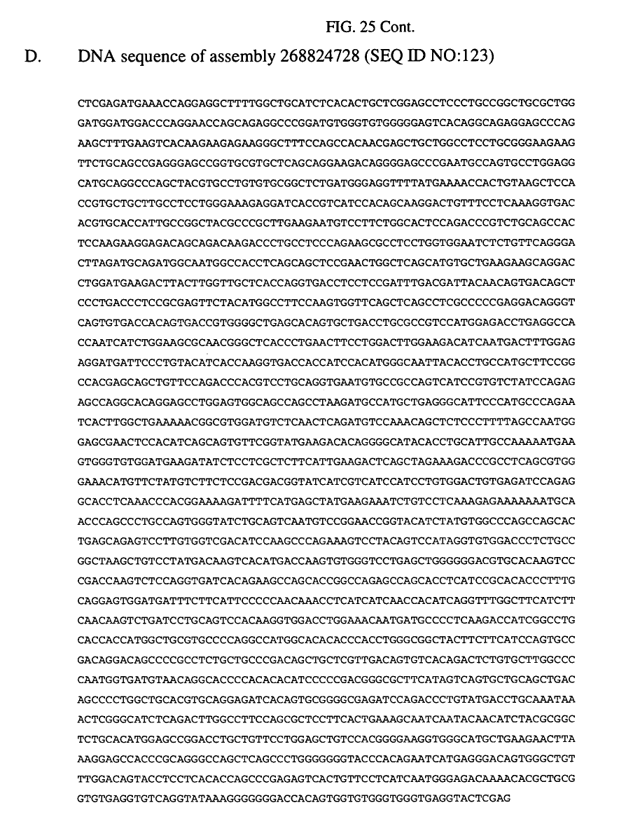

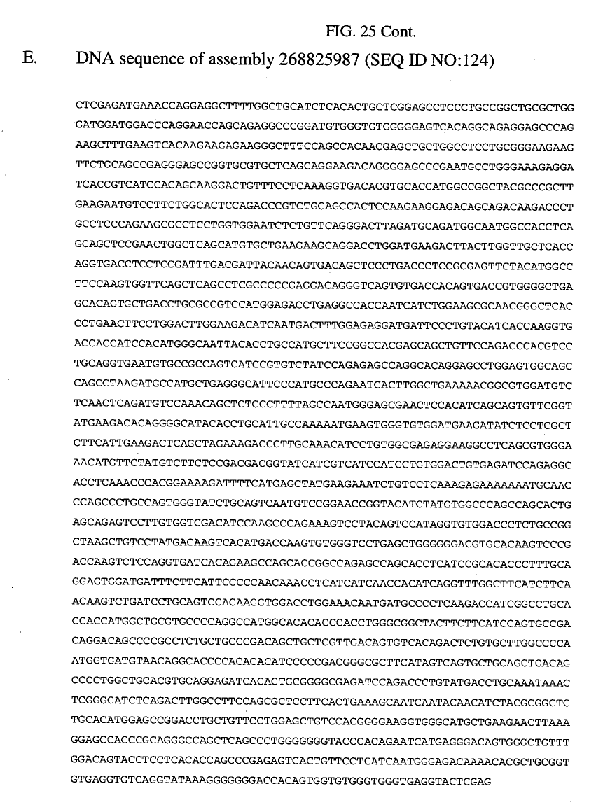

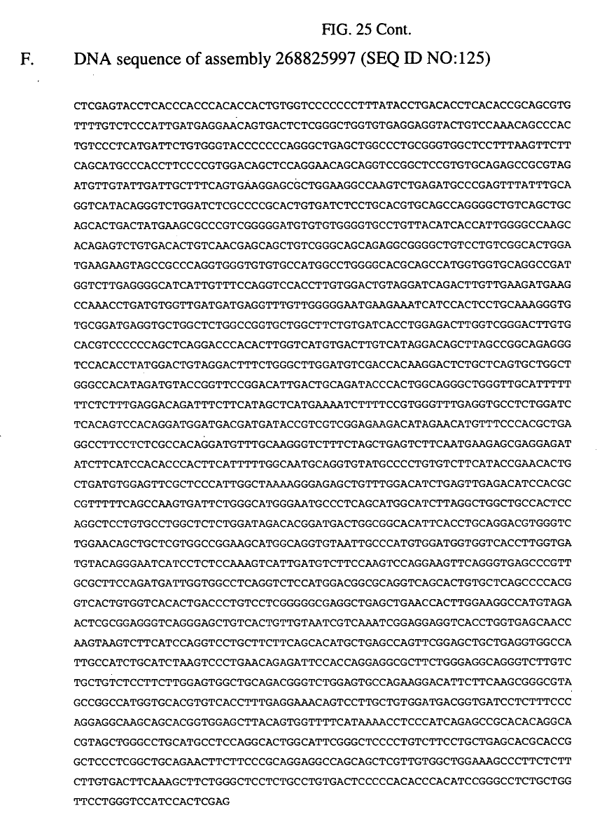

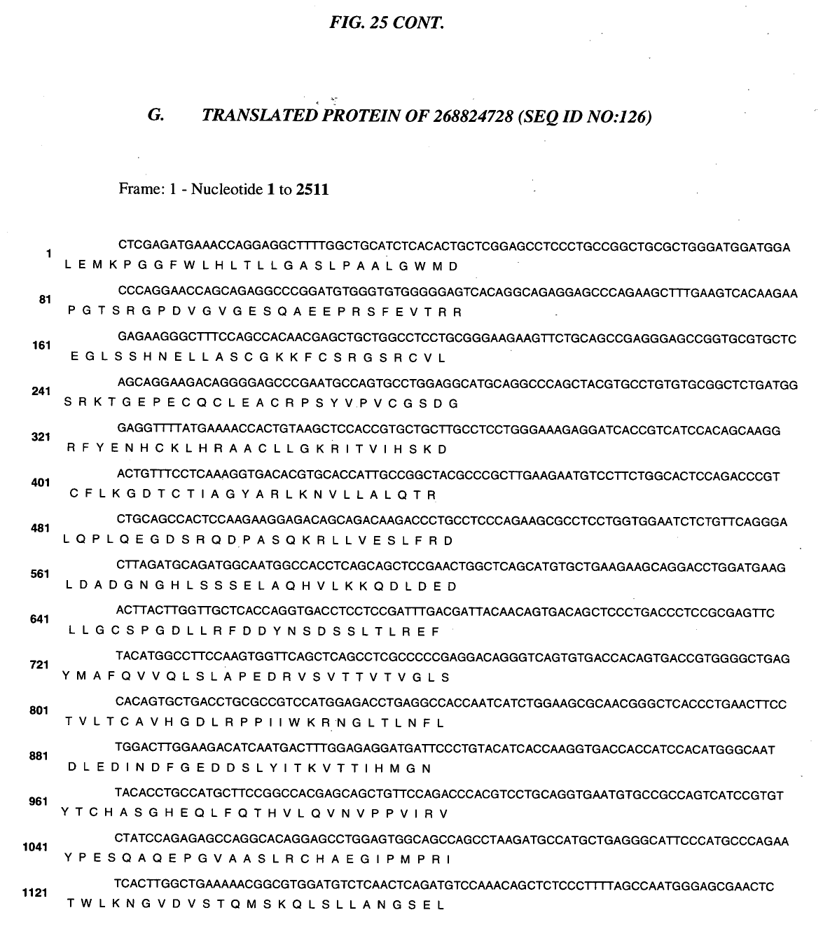

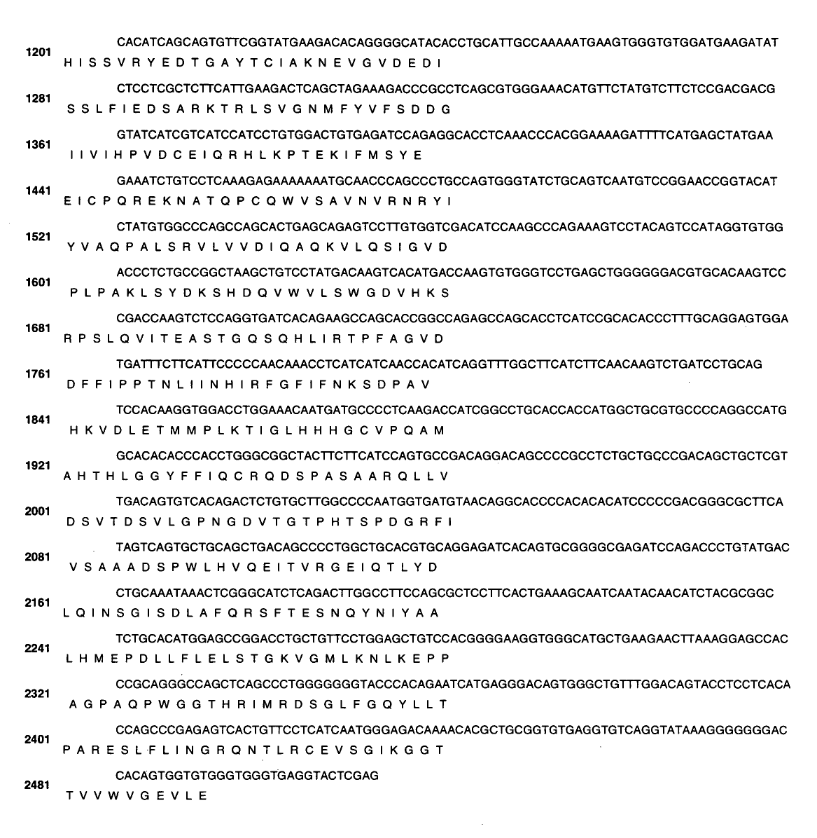

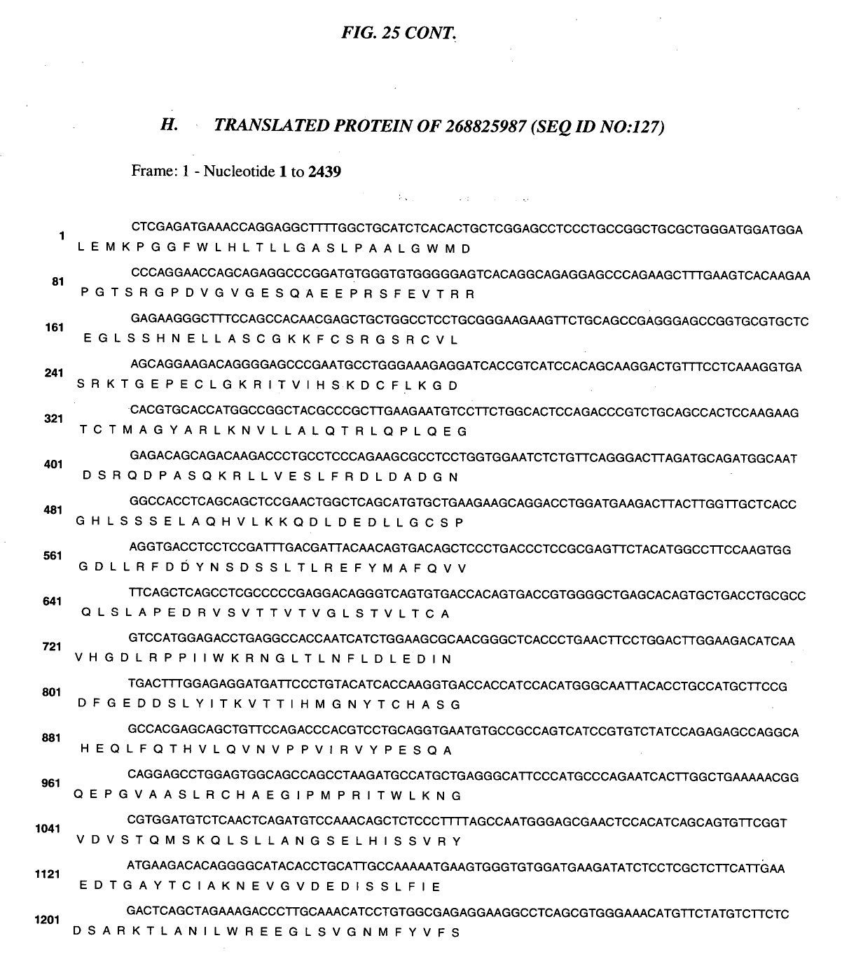

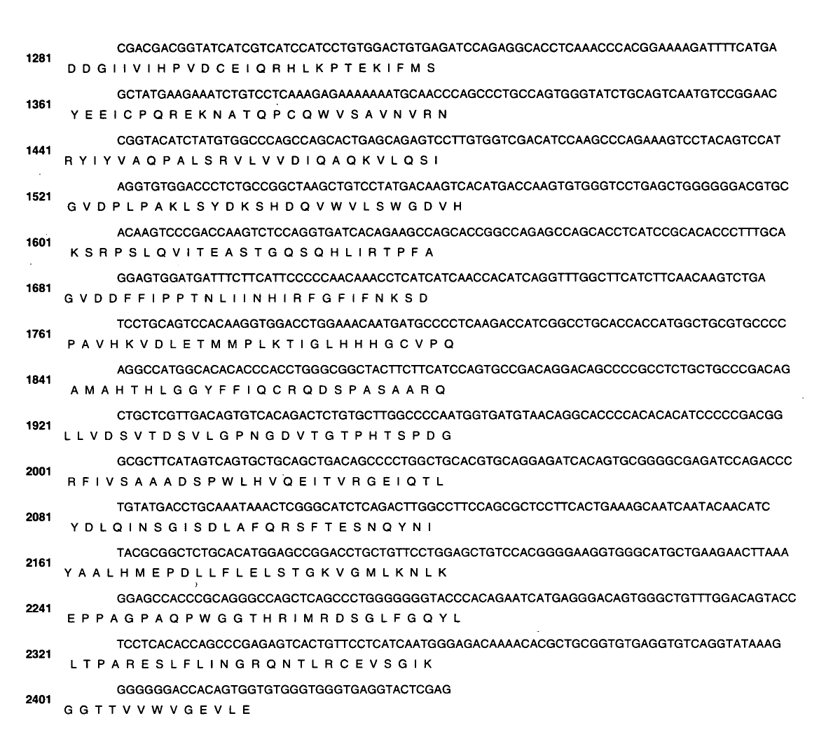

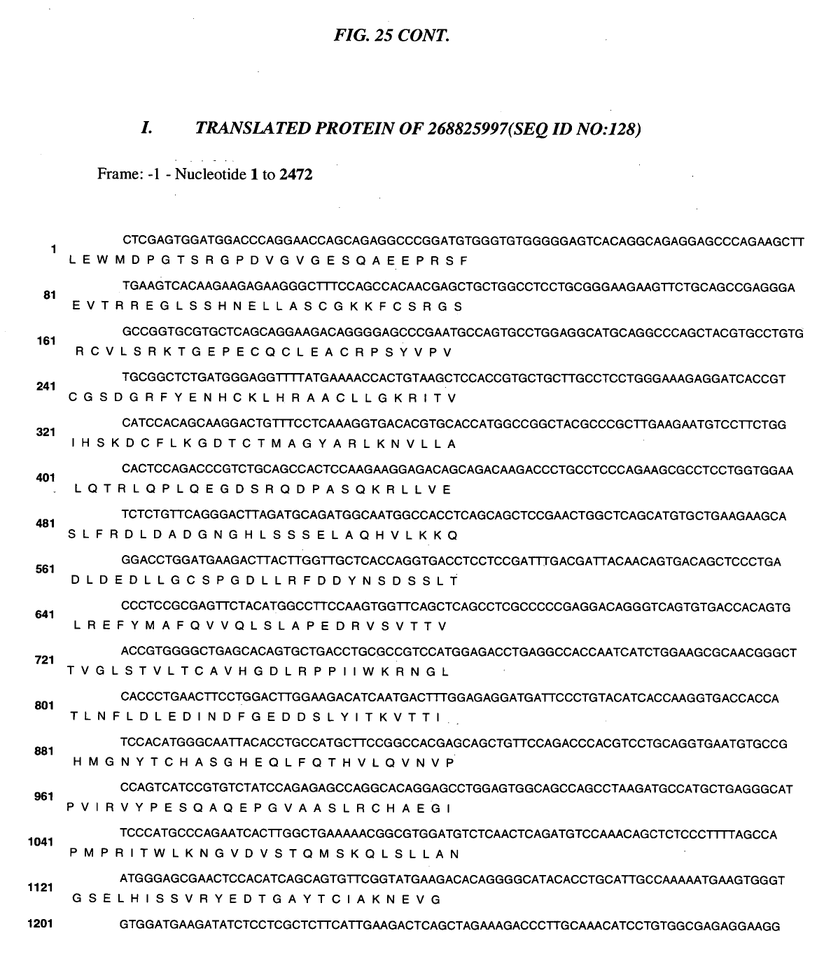

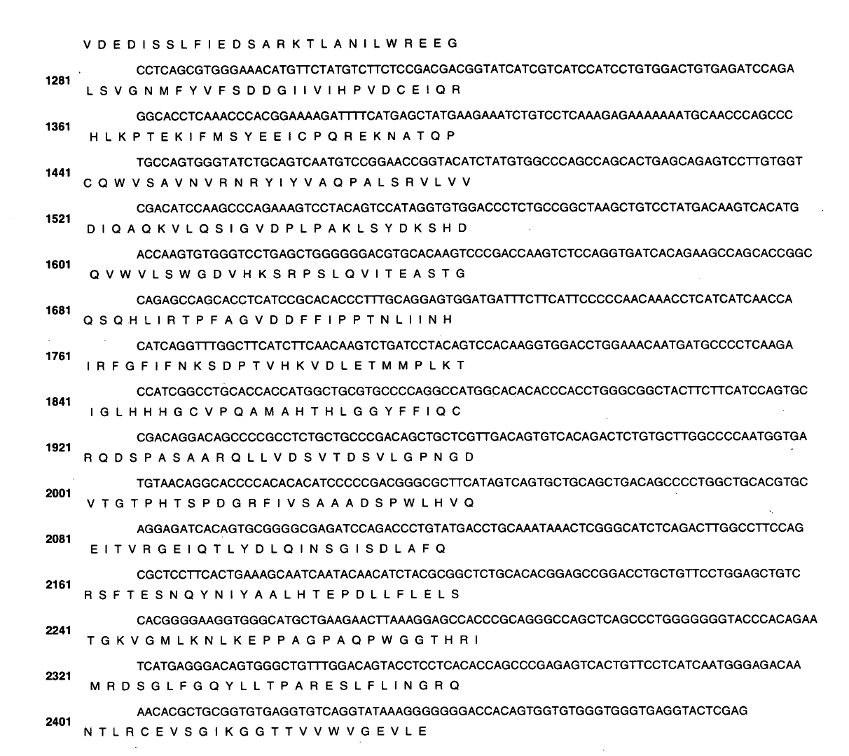

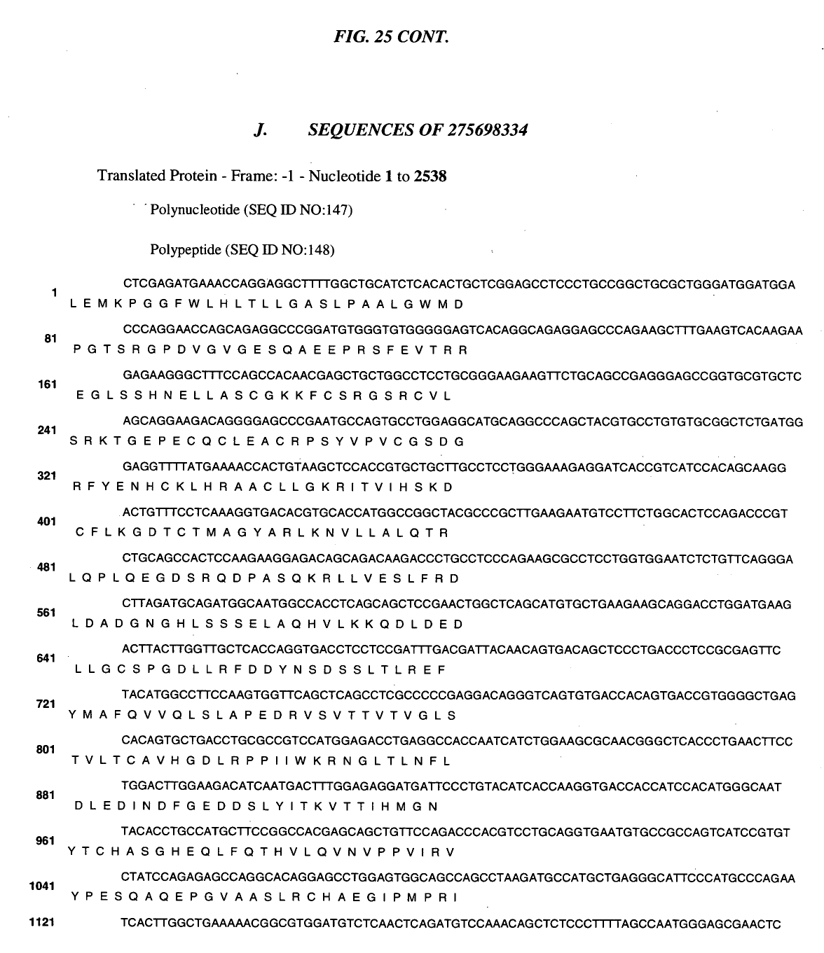

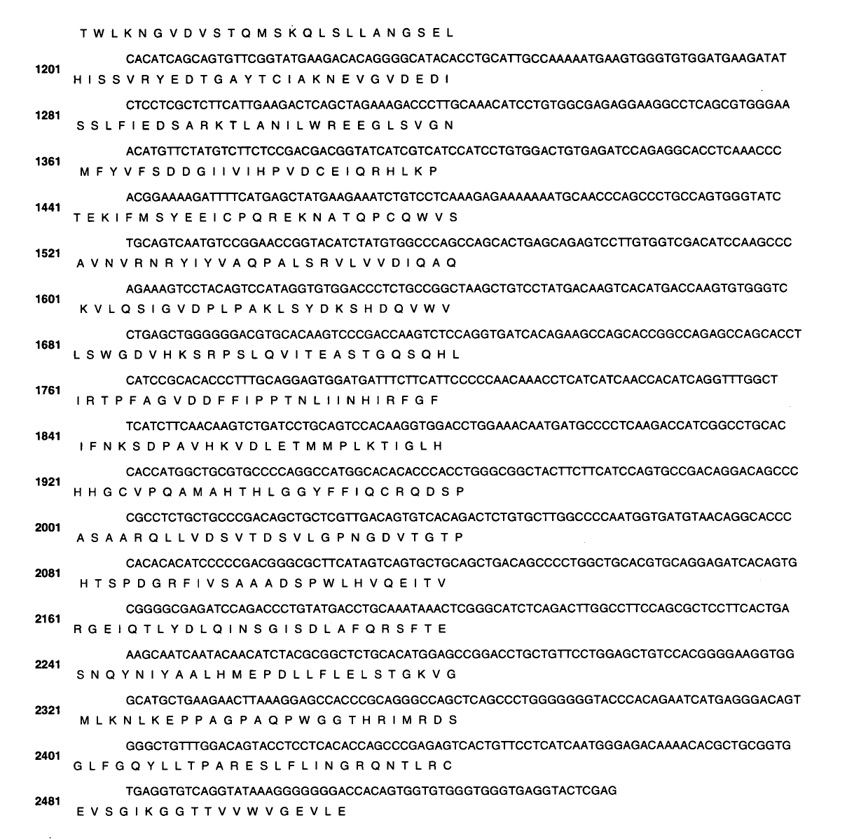

The insert assembly 259341359 shown at FIG. 25b was found to encode an open reading frame between residues 1 and 842 of the target sequence of CG52643-02, shown at FIG. 25a. The cloned insert is 100% identical to the original sequence. A comparison of the polypeptide sequences of CG52643-02 and 259341359 is shown at FIG. 25c.

Other inserts encoding CG52643-02 were sequenced using vector-specific M13 Forward and M13 Reverse primers, and gene-specific primers as described above. For assemblies 268824728 and 268825987 the following gene-specific primers were used:

| | | |
|---|---|---|
| | | SEQ ID |
| Primers | Sequences | NO: |

| | | |
|---|---|---|
| SF1 | 5′-GAAAACCACTGTAAGCTCCACCG-3′ | 95 |
| SF2 | 5′-AGGTGACCTCCTCCGATTTGACGATTA-3′ | 96 |
| SF3 | 5′-TACATCACCAAGGTGACCACCATCC-3′ | 97 |
| SF4 | 5′-GAAGATATCTCCTCGCTCTTCATTG-3′ | 98 |
| SF5 | 5′-CAAGCCCAGAAAGTCCTACAGTCCA-3′ | 99 |
| SF6 | 5′-ACAATGATGCCCCTCAAGACCAT-3′ | 100 |
| SF7 | 5′-CAATCAATACAACATCTACGCGGCT-3′ | 101 |
| SR1 | 5′-TTTCATAAAACCTCCCATCAGAGCC-3′ | 102 |
| SR2 | 5′-CAAGTAAGTCTTCATCCAGGTCCTGCT-3′ | 103 |
| SR3 | 5′-GTGTAATTGCCCATGTGGATGGT-3′ | 104 |
| SR4 | 5′-TATCTTCATCCACACCCACTTCATT-3′ | 105 |
| SR5 | 5′-GATGTCGACCACAAGGACTCTGC-3′ | 106 |
| SR6 | 5′-GGTCTTGAGGGGCATCATTGTTT-3′ | 107 |
| SR7 | 5′-TGATTGCTTTCAGTGAAGGAGCG-3′ | 108 |

For assembly 268825997 the following gene-specific primers were used:

| | | SEQ ID |
|---|---|---|
| Primers | Sequences | NO: |
| SF1 | 5′-CACAGCAAGGACTGTTTCCTCAA-3′ | 109 |
| SF2 | 5′-CTGACCCTCCGCGAGTTCTACAT-3′ | 110 |
| SF3 | 5′-GCCAGTCATCCGTGTCTATCCAGAG-3′ | 111 |
| SF4 | 5′-AAGACCCTTGCAAACATCCTGTG-3′ | 112 |
| SF5 | 5′-CTCTGCCGGCTAAGCTGTCCTA-3′ | 113 |
| SF6 | 5′-ACAATGATGCCCCTCAAGACCAT-3′ | 114 |
| SF7 | 5′-CAATCAATACAACATCTACGCGGCT-3′ | 115 |
| SR1 | 5′-ATGACGGTGATCCTCTTTCCCAG-3′ | 116 |
| SR2 | 5′-AGCTGTCACTGTTGTAATCGTCAAAT-3′ | 117 |
| SR3 | 5′-CAGGTGTAATTGCCCATGTGGAT-3′ | 118 |
| SR4 | 5′-GGGTCTTTCTAGCTGAGTCTTCAATG-3′ | 119 |
| SR5 | 5′-TATGGACTGTAGGACTTTCTGGGCT-3′ | 120 |
| SR6 | 5′-ATGGTCTTGAGGGGCATCATTGT-3′ | 121 |
| SR7 | 5′-TGATTGCTTTCAGTGAAGGAGCG-3′ | 122 |

The insert assemblies 268824728 (SEQ ID NO: 123 and SEQ ID NO:126) and 268825987 (SEQ ID NO: 124 and SEQ ID NO:127) were found to encode an open reading frame between residues 1 and 842, and assembly 268825997 (SEQ ID NO: 125 and SEQ ID NO: 128) was found to encode an open reading frame between residues 23 and 842 of the target sequence of CG52643-02. The protein associated with 268825997 is encoded in a negative reading frame. The sequence shown has been reverse-complemented and renumbered to allow reading of the protein in the expected N to C direction. The cloned inserts differ from the original sequence. Assembly 268824728 has a deletion of 9 amino acids (439-447) and two amino acid changes: M4331 and T757M. Assembly 268825987 has a 33 amino acid deletion (88-120) and one amino acid change: T757M. Assembly 268825997 one amino acid change: A620T.

For assembly 275698334 the following gene-specific primers were used:

| | | SEQ ID |
|---|---|---|
| Primers | Sequences | NO: |
| SF1 | 5′-GAAAACCACTGTAAGCTCCACCG-3′ | 131 |
| SF2 | 5′-AGGTGACCTCCTCCGATTTGACGATTA-3′ | 132 |
| SF3 | 5′-TACATCACCAAGGTGACCACCATCC-3′ | 133 |
| SF4 | 5′-GAAGATATCTCCTCGCTCTTCATTG-3′ | 134 |
| SF5 | 5′-CAAGCCCAGAAAGTCCTACAGTCCA-3′ | 135 |
| SF6 | 5′-ACAATGATGCCCCTCAAGACCAT-3′ | 136 |
| SF7 | 5′-CAATCAATACAACATCTACGCGGCT-3′ | 137 |
| SR1 | 5′-TTTCATAAAACCTCCCATCAGAGCC-3′ | 138 |
| SR2 | 5′-CAAGTAAGTCTTCATCCAGGTCCTGCT-3′ | 139 |
| SR3 | 5′-GTGTAATTGCCCATGTGGATGGT-3′ | 140 |
| SR4 | 5′-TATCTTCATCCACACCCACTTCATT-3′ | 141 |
| SR5 | 5′-GATGTCGACCACAAGGACTCTGC-3′ | 142 |
| SR6 | 5′-GGTCTTGAGGGGCATCATTGTTT-3′ | 143 |
| SR7 | 5′-TGATTGCTTTCAGTGAAGGAGCG-3′ | 144 |

The insert assembly 275698334 was found to encode an open reading frame between residues 1 and 842 of the target sequence of CG52643-02. The cloned insert differs from the original sequence at one position: T757M. The protein associated with 275698334 is encoded in a negative reading frame. The sequence shown below has been reverse-complemented and renumbered to allow reading of the protein in the expected N to C direction.

Printed 80 characters to a line.

### EQUIVALENTS

From the foregoing detailed description of the specific embodiments of the invention, it should be apparent that unique nucleotides, polypeptides, and methods of use thereof for the SECX genes have been described. Although particular embodiments have been disclosed herein in detail, this has been done by way of example for purposes of illustration only, and is not intended to be limiting with respect to the scope of the appended claims which follow. In particular, it is contemplated by the inventor that various substitutions, alterations, and modifications may be made to the invention without departing from the spirit and scope of the invention as defined by the claims. For instance, the choice of which SECX nucleotide or polypeptide or method of use thereof is believed to be a matter of routine for a person of ordinary skill in the art with knowledge of the embodiments described herein.

1. An isolated polypeptide comprising an amino acid sequence selected from the group consisting of:
a) a mature form of an amino acid sequence chosen from the group consisting of SEQ ID NOs: 2, 4, 6, 8, 10, 12, 14, 16, 18, 20, 22, 24, 26, 28, 30, 87, 88, 126, 127, 145, 146, 148, and 150;
b) a variant of a mature form of an amino acid sequence selected from the group consisting of SEQ ID NOs: 2, 4, 6, 8, 10, 12, 14, 16, 18, 20, 22, 24, 26, 28, 30, 87, 88, 126, 127, 145, 146, 148, and 150, wherein any amino acid in the mature form of the chosen sequence is changed to a different amino acid, provided that no more than 15% of the amino acid residues in the sequence of the mature form are so changed;
c) an amino acid sequence selected from the group consisting of SEQ ID NOs: 2, 4, 6, 8, 10, 12, 14, 16, 18, 20, 22, 24, 26, 28, 30, 87, 88, 126, 127, 145, 146, 148, and 150; and
d) a variant of an amino acid sequence selected from the group consisting of SEQ ID NOs: 2, 4, 6, 8, 10, 12, 14, 16, 18, 20, 22, 24, 26, 28, 30, 87, 88, 126, 127, 145, 146, 148, and 150, in which any amino acid specified in the chosen sequence is changed to a different amino acid, provided that no more than 15% of the amino acid residues in the sequence are so changed.

2. The polypeptide of claim 1 that is a variant polypeptide, wherein the polypeptide comprises the amino acid sequence of a naturally occurring allelic variant of said polypeptide.

3. The polypeptide of claim 2, wherein the variant is the translation of a single nucleotide polymorphism.

4. The polypeptide of claim 1, wherein any amino acid specified in the chosen sequence is changed to provide a conservative substitution.

5. An isolated polypeptide comprising an amino acid sequence selected from the group consisting of:
a) a mature form of an amino acid sequence chosen from the group consisting of SEQ ID NOs: 2, 4, 6, 8, 10, 12, 14, 16, 18, 20, 22, 24, 26, 28, 30, 87, 88, 126, 127, 145, 146, 148, and 150; and
b) an amino acid sequence selected from the group consisting of SEQ ID NOs: 2, 4, 6, 8, 10, 12, 14, 16, 18, 20, 22, 24, 26, 28, 30, 87, 88, 126, 127, 145, 146, 148, and 150.

6. An isolated nucleic acid molecule comprising a nucleic acid sequence encoding a polypeptide comprising the amino acid sequence of a polypeptide selected from the group consisting of:
a) a mature form of an amino acid sequence selected from the group consisting of SEQ ID NOs: 2, 4, 6, 8, 10, 12, 14, 16, 18, 20, 22, 24, 26, 28, 30, 87, 88, 126, 127, 145, 146, 148, and 150;
b) a mature form of a variant of an amino acid sequence selected from the group consisting of SEQ ID NOs: 2, 4, 6, 8, 10, 12, 14, 16, 18, 20, 22, 24, 26, 28, 30, 87, 88, 126, 127, 145, 146, 148, and 150, wherein any amino acid in the mature form of the chosen sequence is changed to a different amino acid, provided that no more than 15% of the amino acid residues in the sequence of the mature form are so changed;
c) an amino acid sequence selected from the group consisting of SEQ ID NOs: 2, 4, 6, 8, 10, 12, 14, 16, 18, 20, 22, 24, 26, 28, 30, 87, 88, 126, 127, 145, 146, 148, and 150;
d) a variant of an amino acid sequence selected from the group consisting of SEQ ID NOs: 2, 4, 6, 8, 10, 12, 14, 16, 18, 20, 22, 24, 26, 28, 30, 87, 88, 126, 127, 145, 146, 148, and 150, in which any amino acid specified in the chosen sequence is changed to a different amino acid, provided that no more than 15% of the amino acid residues in the sequence are so changed; and
e) a nucleic acid fragment encoding at least a portion of a polypeptide comprising an amino acid sequence chosen from the group consisting of SEQ ID NOs: 2, 4, 6, 8, 10, 12, 14, 16, 18, 20, 22, 24, 26, 28, 30, 87, 88, 126, 127, 145, 146, 148, and 150;
or the complement of said nucleic acid molecule.

7. The nucleic acid molecule of claim 6, wherein the nucleic acid molecule comprises the nucleotide sequence of a naturally occurring allelic nucleic acid variant.

8. The nucleic acid molecule of claim 6 that encodes a variant polypeptide, wherein the encoded variant polypeptide has the polypeptide sequence of a naturally occurring polypeptide variant.

9. The nucleic acid molecule of claim 6, wherein the nucleic acid molecule comprises a single nucleotide polymorphism encoding said variant polypeptide.

10. The nucleic acid molecule of claim 6, wherein said nucleic acid molecule comprises a nucleotide sequence selected from the group consisting of
a) a nucleotide sequence comprising a nucleotide sequence selected from the group consisting of SEQ ID NOs: 1,3,5,7,9,11,13,15,17, 19, 21, 23, 25, 27, 29, 85, 86, 123, 124, 125, 144, 147 and 149;
b) a nucleotide sequence wherein one or more nucleotides in a nucleotide sequence from the group consisting of SEQ ID NOs: 1,3,5,7,9,11,13,15,17, 19, 21, 23, 25, 27, 29, 85, 86, 123, 124, 125, 144, 147 and 149 is changed from that given by the chosen sequence to a different nucleotide provided that no more than 20% of the nucleotides are so changed;
c) a nucleic acid fragment of a); and
d) a nucleic acid fragment of b).

11. The nucleic acid molecule of claim 6, wherein said nucleic acid molecule hybridizes under stringent conditions to a nucleotide sequence chosen from the group consisting of SEQ ID NOs: 1,3,5,7,9,11,13,15,17, 19, 21, 23, 25, 27, 29, 85, 86, 123, 124, 125, 144, 147 and 149, or a complement of said nucleotide sequence.

12. The nucleic acid molecule of claim 6, wherein the nucleic acid molecule comprises a nucleotide sequence in which any nucleotide specified in the coding sequence of the chosen nucleotide sequence is changed from that given by the chosen sequence to a different nucleotide provided that no more than 20% of the nucleotides in the chosen coding sequence are so changed, an isolated second polynucleotide that is a complement of the first polynucleotide, or a fragment of any of them.

13. An isolated nucleic acid molecule comprising a nucleic acid sequence encoding a polypeptide comprising the amino acid sequence of a polypeptide selected from the group consisting of:
a) a mature form of an amino acid sequence selected from the group consisting of SEQ ID NOs: SEQ ID NOs: 2, 4, 6, 8, 10, 12, 14, 16, 18, 20, 22, 24, 26, 28, 30, 87, 88, 126, 127, 145, 146, 148, and 150;
b) an amino acid sequence selected from the group consisting of SEQ ID NOs: 2, 4, 6, 8, 10, 12, 14, 16, 18, 20, 22, 24, 26, 28, 30, 87, 88, 126, 127, 145, 146, 148, and 150; and
c) a nucleic acid fragment encoding at least a portion of a polypeptide comprising an amino acid sequence chosen from the group consisting of SEQ ID NOs: 2, 4, 6, 8, 10, 12, 14, 16, 18, 20, 22, 24, 26, 28, 30, 87, 88, 126, 127, 145, 146, 148, and 150;
or the complement of said nucleic acid molecule.

14. The nucleic acid molecule of claim 13, wherein said nucleic acid molecule comprises a nucleotide sequence selected from the group consisting of a) a nucleotide sequence comprising a nucleotide sequence selected from the group consisting of SEQ ID NOs: 1,3,5,7,9,11,13,15,17, 19, 21, 23, 25, 27, 29, 85, 86, 123, 124, 125, 144, 147 and 149; and
b) a nucleic acid fragment of a), or the complement of said nucleic acid molecule.

15. A vector comprising the nucleic acid molecule of claim 6.

16. The vector of claim 15, further comprising a promoter operably linked to said nucleic acid molecule.

17. A vector comprising the nucleic acid molecule of claim 13.

18. The vector of claim 16, further comprising a promoter operably linked to said nucleic acid molecule.

19. A cell comprising the vector of claim 15.

20. A cell comprising the vector of claim 17.

21. An antibody that binds immunospecifically to the polypeptide of claim 1.

22. The antibody of claim 21, wherein said antibody is a monoclonal antibody.

23. The antibody of claim 21, wherein the antibody is a humanized antibody.

24. An antibody that binds immunospecifically to the polypeptide of claim 5.

25. The antibody of claim 24, wherein said antibody is a monoclonal antibody.

26. The antibody of claim 24, wherein the antibody is a humanized antibody.

27. A method for determining the presence or amount of the polypeptide of claim 1 in a sample, the method comprising:
(a) providing the sample;
(b) contacting the sample with an antibody that binds immunospecifically to a polypeptide of claim 1; and
(c) determining the presence or amount of antibody bound to said polypeptide,
thereby determining the presence or amount of polypeptide in said sample.

28. A method for determining the presence or amount of the polypeptide of claim 5 in a sample, the method comprising:
(a) providing the sample;
(b) contacting the sample with an antibody that binds immunospecifically to a polypeptide of claim 5; and
(c) determining the presence or amount of antibody bound to said polypeptide,
thereby determining the presence or amount of polypeptide in said sample.

29. A method for determining the presence or amount of the nucleic acid molecule of claim 6 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

30. A method for determining the presence or amount of the nucleic acid molecule of claim 13 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

31. A method of identifying an agent that binds to a polypeptide of claim 1, the method comprising:
(a) contacting said polypeptide with said agent; and
(b) determining whether said agent binds to said polypeptide.

32. A method of identifying an agent that binds to a polypeptide of claim 5, the method comprising:
(a) contacting said polypeptide with said agent; and
(b) determining whether said agent binds to said polypeptide.

33. A method for identifying a potential therapeutic agent for use in treatment of a pathology, wherein the pathology is related to aberrant expression or aberrant physiological interactions of the polypeptide of claim 1, the method comprising:
(a) identifying a polypeptide related to the pathology;
(b) providing a cell expressing the chosen polypeptide and having a property or function due to the action of the polypeptide;
(c) contacting the cell with a composition comprising a candidate substance, and
(d) determining whether the substance alters the property or function due to the action of the polypeptide;
whereby, if the alteration observed in the presence of the substance is not observed when the cell is contacted with a composition devoid of the substance, the substance is identified as a potential therapeutic agent.

34. A method for identifying a potential therapeutic agent for use in treatment of a pathology, wherein the pathology is related to aberrant expression or aberrant physiological interactions of the polypeptide of claim 5, the method comprising:
(a) identifying a polypeptide related to the pathology;
(b) providing a cell expressing the chosen polypeptide and having a property or function due to the action of the polypeptide;
(c) contacting the cell with a composition comprising a candidate substance, and
(d) determining whether the substance alters the property or function due to the action of the polypeptide;
whereby, if the alteration observed in the presence of the substance is not observed when the cell is contacted with a composition devoid of the substance, the substance is identified as a potential therapeutic agent.

35. A method for modulating the activity of the polypeptide of claim 1, the method comprising contacting a cell sample expressing the polypeptide of said claim with a compound that binds to said polypeptide in an amount sufficient to modulate the activity of the polypeptide.

36. A method for modulating the activity of the polypeptide of claim 5, the method comprising contacting a cell sample expressing the polypeptide of said claim with a compound that binds to said polypeptide in an amount sufficient to modulate the activity of the polypeptide.

37. A method of treating or preventing a SECX-associated disorder, said method comprising administering to a subject in which such treatment or prevention is desired the polypeptide of claim 1 in an amount sufficient to treat or prevent said SECX-associated disorder in said subject.

38. The method of claim 37, wherein said subject is a human.

39. A method of treating or preventing a SECX-associated disorder, said method comprising administering to a subject in which such treatment or prevention is desired the polypeptide of claim 5 in an amount sufficient to treat or prevent said SECX-associated disorder in said subject.

40. The method of claim 40, wherein said subject is a human.

41. A method of treating or preventing a SECX-associated disorder, said method comprising administering to a subject in which such treatment or prevention is desired the nucleic acid of claim 6 in an amount sufficient to treat or prevent said SECX-associated disorder in said subject.

42. The method of claim 41, wherein said subject is a human.

43. A method of treating or preventing a SECX-associated disorder, said method comprising administering to a subject in which such treatment or prevention is desired the nucleic acid of claim 13 in an amount sufficient to treat or prevent said SECX-associated disorder in said subject.

44. The method of claim 43, wherein said subject is a human.

45. A method of treating or preventing a SECX-associated disorder, said method comprising administering to a subject in which such treatment or prevention is desired the antibody of claim 21 in an amount sufficient to treat or prevent said SECX-associated disorder in said subject.

46. The method of claim 45, wherein the subject is a human.

47. A pharmaceutical composition comprising the polypeptide of claim 1 and a pharmaceutically acceptable carrier.

48. A pharmaceutical composition comprising the polypeptide of claim 5 and a pharmaceutically acceptable carrier.

49. A pharmaceutical composition comprising the nucleic acid molecule of claim 6 and a pharmaceutically acceptable carrier.

50. A pharmaceutical composition comprising the nucleic acid molecule of claim 13 and a pharmaceutically acceptable carrier.

51. A pharmaceutical composition comprising the antibody of claim 21 and a pharmaceutically acceptable carrier.

52. A pharmaceutical composition comprising the antibody of claim 24 and a pharmaceutically acceptable carrier.

53. A kit comprising in one or more containers, the pharmaceutical composition of claim 47.

54. A kit comprising in one or more containers, the pharmaceutical composition of claim 48.

55. A kit comprising in one or more containers, the pharmaceutical composition of claim 49.

56. A kit comprising in one or more containers, the pharmaceutical composition of claim 50.

57. A kit comprising in one or more containers, the pharmaceutical composition of claim 51.

58. A kit comprising in one or more containers, the pharmaceutical composition of claim 52.

59. The use of a therapeutic in the manufacture of a medicament for treating a syndrome associated with a human disease, the disease selected from a SECX-associated disorder, wherein said therapeutic is selected from the group consisting of a SECX polypeptide, a SECX nucleic acid, and a SECX antibody.

60. A method for screening for a modulator of activity or of latency or predisposition to a SECX-associated disorder, said method comprising:
a) administering a test compound to a test animal at increased risk for a SECX-associated disorder, wherein said test animal recombinantly expresses the polypeptide of claim 1;
b) measuring the activity of said polypeptide in said test animal after administering the compound of step (a);
c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a SECX-associated disorder.

61. The method of claim 59, wherein said test animal is a recombinant test animal that expresses a test protein transgene or expresses said transgene under the control of a promoter at an increased level relative to a wild-type test animal, and wherein said promoter is not the native gene promoter of said transgene.

62. A method for screening for a modulator of activity or of latency or predisposition to a SECX-associated disorder, said method comprising:
a) administering a test compound to a test animal at increased risk for a SECX-associated disorder, wherein said test animal recombinantly expresses the polypeptide of claim 5;
b) measuring the activity of said polypeptide in said test animal after administering the compound of step (a);
c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a SECX-associated disorder.

63. The method of claim 62, wherein said test animal is a recombinant test animal that expresses a test protein transgene or expresses said transgene under the control of a promoter at an increased level relative to a wild-type test animal, and wherein said promoter is not the native gene promoter of said transgene.

64. A method for determining the presence of or predisposition to a disease associated with altered levels of the polypeptide of claim 1 in a first mammalian subject, the method comprising:
a) measuring the level of expression of the polypeptide in a sample from the first mammalian subject; and
b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

65. A method for determining the presence of or predisposition to a disease associated with altered levels of the polypeptide of claim 5 in a first mammalian subject, the method comprising:
a) measuring the level of expression of the polypeptide in a sample from the first mammalian subject; and
b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

66. A method for determining the presence of or predisposition to a disease associated with altered levels of the nucleic acid molecule of claim 6 in a first mammalian subject, the method comprising:
a) measuring the amount of the nucleic acid in a sample from the first mammalian subject; and
b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

67. A method for determining the presence of or predisposition to a disease associated with altered levels of the nucleic acid molecule of claim 13 in a first mammalian subject, the method comprising:
a) measuring the amount of the nucleic acid in a sample from the first mammalian subject; and
b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

68. A method of treating a pathological state in a mammal, the method comprising administering to the mammal a polypeptide in an amount that is sufficient to alleviate the pathological state, wherein the polypeptide is a polypeptide having an amino acid sequence at least 95% identical to the polypeptide of claim 1, or a biologically active fragment thereof.

69. A method of treating a pathological state in a mammal, the method comprising administering to the mammal a polypeptide in an amount that is sufficient to alleviate the pathological state, wherein the polypeptide is a polypeptide having an amino acid sequence at least 95% identical to the polypeptide of claim 5, or a biologically active fragment thereof.

70. A method of treating a pathological state in a mammal, the method comprising administering to the mammal the antibody of claim 21 in an amount sufficient to alleviate the pathological state.

71. A method of treating a pathological state in a mammal, the method comprising administering to the mammal the antibody of claim 24 in an amount sufficient to alleviate the pathological state.
